(19) United States
(12) Patent Application Publication
(10) Pub. No.: US 2026/0295128 A1
(43) Pub. Date: Oct. 1, 2026

(54) SOFT AND STRETCHABLE MICROELECTRODES WITH TUNABLE ANISOTROPIC PROPERTIES

(71) Applicant: The Research Foundation for The State University of New York, Binghamton, NY (US)

(72) Inventors: Siyuan Rao, Binghamton, NY (US); Qianbin Wang, Binghamton, NY (US); Sizhe Huang, Binghamton, NY (US); Ruobai Xiao, Binghamton, NY (US)

(21) Appl. No.: 19/567,446

(22) Filed: Mar. 16, 2026

Related U.S. Application Data
(60) Provisional application No. 63/772,623, filed on Mar. 16, 2025.

(51) Int. Cl. A61L 31/14; A61L 31/04; A61L 31/10

(52) U.S. Cl. CPC A61L 31/145; A61L 31/048; A61L 31/10; A61L 2400/12

(57) ABSTRACT

A hydrogel electrode for electrically interfacing with animal tissue, comprising with a chemically crosslinked polymer matrix configured having a diameter of less than 500 μm, having anisotropically aligned conductive nanorods or nanotubes, surrounded by a tubular insulating layer. A method of making a soft, elastic, conductive hydrogel electrode, comprising: combining a hydrogel-forming polymer with carbon nanotubes and a carbon nanotube dispersing agent to form a heterogeneous mixture; crosslinking the hydrogel-forming polymer to form an elongated hydrogel having a diameter less than 1 mm; and applying a cyclic strain on the molded hydrogel to align the carbon nanotubes with an elongation axis of the molded hydrogel to form an anisotropically-conductive hydrogel.

### CROSS REFERENCE TO RELATED APPLICATIONS

This application claims priority to U.S. Provisional Application No. 63/772,623, filed on Mar. 16, 2025.

### STATEMENT OF GOVERNMENT INTEREST

The invention was made with government support under 2239030 and R00MH120279 awarded by the National Science Foundation. The government has certain rights in the invention.

### FIELD OF THE INVENTION

The present invention relates to the field of microelectrodes for insertion into neural tissue, and more particularly, to hydrogel electrodes for obtaining central nervous system recordings.

### INCORPORATION BY REFERENCE AND INTERPRETATION OF LANGUAGE

Citation or identification of any reference herein, in any section of this application, shall not be construed as an admission that such reference is necessarily available as prior art to the present application. The disclosures of each reference disclosed herein, whether U.S. or foreign patent literature, or non-patent literature, are hereby incorporated by reference in their entirety in this application, and shall be treated as if the entirety thereof forms a part of this application.

All cited or identified references are provided for their disclosure of technologies to enable practice of the present invention, to provide basis for claim language, and to make clear applicant's possession of the invention with respect to the various aggregates, combinations, and subcombinations of the respective disclosures or portions thereof (within a particular reference or across multiple references). The citation of references is intended to be part of the disclosure of the invention, and not merely supplementary background information. The incorporation by reference does not extend to teachings which are inconsistent with the invention as expressly described herein (which may be treated as counter examples), and is evidence of a proper interpretation by persons of ordinary skill in the art of the terms, phrase and concepts discussed herein, without being limiting as the sole interpretation available.

The present specification is not to be interpreted by recourse to lay dictionaries in preference to field-specific dictionaries or usage. Where a conflict of interpretation exists, the hierarchy of resolution shall be the express specification, references cited for propositions, incorporated references generally, the inventors' prior publications relating to the field, academic literature in the field generally, commercial literature in the field, field-specific dictionaries, lay literature in the field, general purpose dictionaries, and common understanding.

### BACKGROUND OF THE INVENTION

Neural interfaces are critical for bridging the nervous system with external devices, enabling precise detection and modulation of complex neural activities, including electrical, chemical, and mechanical signaling at the cellular level. However, conventional neural interfaces have faced significant challenges related to tissue compatibility, multifunctionality, and long-term stability. These challenges were primarily attributed to mechanical mismatches between the interfaces and neural tissues, leading to foreign body responses and reduced functionality over time. Additionally, the lack of multifunctionality limited their ability to fully capture and interact with the intricate signaling dynamics of the nervous system.

The mechanical mismatch between neural interfaces and neural tissues presents a critical challenge in the design and functionality of neural devices. This discrepancy in mechanical properties can result in various adverse effects, including tissue damage, immune activation, and chronic inflammation, all of which ultimately compromise the long-term stability and effectiveness of neural interfaces. The concept of device adaptivity highlights the need for mechanical compatibility between neural probes and the surrounding nervous system. Specifically, when implants do not match the soft, flexible nature of neural tissues, they fail to accommodate the natural micromovements of the brain or peripheral nerves, leading to significant complications.

Traditional neural devices are frequently constructed from stiff materials such as silicon or metals like platinum and tungsten, which have vastly different mechanical properties compared to neural tissues. For example, silicon has a modulus of elasticity in the range of 5-20 GPa, while metals such as platinum and tungsten are similarly rigid. In stark contrast, the modulus of neural tissues, such as brain tissue, typically falls between 1-10 kPa. This disparity creates a rigid interface within a highly dynamic environment, where continuous movements of neural tissue relative to the implant exert strain on the device-tissue interface, causing neuronal drift, shear injury, and eliciting immune responses.

The micromotion of neural tissues, particularly in the brain, can result from physiological processes such as heartbeat and respiration, causing tissue displacement on the order of 4-30 μm in rodent models⁸⁵. Over time, such mechanical strain exacerbates the formation of glial scars—accumulations of astrocytes and microglia that encapsulate the device—isolating it from surrounding neurons, thereby diminishing signal quality and rendering the device less effective.

The immune response triggered by mechanical mismatch also leads to chronic inflammation. Stiff, unyielding implants create friction and irritation as neural tissues move around them, stimulating astrocytes and microglia to form glial scars. These scars effectively encapsulate the device, isolating it from nearby neurons and reducing the efficacy of the neural interface for recording and stimulation. Over time, this inflammatory response further compromises device function, contributing to a hostile microenvironment marked by ongoing inflammation and neuronal damage near the implant.

Recent advances in polymer-based neural probes aim to mitigate these mechanical mismatches, yet they remain suboptimal for complete compatibility with neural tissue. Polymers such as polyimide, PDMS and parylene exhibit elastic moduli in the range of 1-5 GPa—significantly softer than traditional silicon or metal probes but still considerably stiffer than neural tissues⁸⁶. While these polymer-based devices reduce mechanical mismatch, they still exert strain during tissue micromotions, posing challenges for long-term implantation, especially in sensitive regions such as the spinal cord, where even minor strain can result in severe outcomes, including paralysis or mortality.

To address these limitations, researchers are investigating ultra-compliant materials such as hydrogels, which have moduli that can be tuned to closely match that of neural tissues. These materials offer promises for minimizing mechanical mismatch in sensitive applications, such as spinal cord injury models, where tissue damage must be minimized. However, the durability, stability, and long-term functionality of such ultra-soft materials remain challenging.

In summary, mechanical mismatch between neural interfaces and neural tissues remains a major barrier in neural interface technology. Advances in materials science are crucial for developing devices that are both biocompatible and mechanically compliant with the soft, dynamic nature of neural tissues. Achieving this compatibility will improve the longevity and efficacy of neural implants, reduce immune responses and tissue damage, and enable the safe and reliable use of neural devices across a wide range of clinical applications.

Hydrogels are defined as water-insoluble polymeric networks with a high capacity for water absorption. Structurally, hydrogels are macromolecular polymer gels composed of a crosslinked network of polymer chains. These materials are synthesized from hydrophilic monomers through either chain or step growth polymerization, often incorporating functional crosslinkers to stabilize the network. The three-dimensional (3D) structure of hydrogels is formed using synthetic or natural polymers, whether homopolymers or copolymers, achieved through molecular entanglements or chemical crosslinking⁹⁶.

The ability of hydrogels to swell in aqueous environments makes them highly suitable for biomedical applications, including drug delivery and tissue engineering. Crosslinking, achieved through either physical or chemical means, imparts a stable 3D network that renders hydrogels insoluble, enabling the immobilization and controlled release of bioactive agents and biomolecules. Due to their high water content and softness, hydrogels closely mimic the properties of natural soft tissues, making them an ideal material for diverse biomedical applications.

Hydrogels are versatile materials whose network structures can be stabilized through various types of physical crosslinking, including ionic interactions. Alginate, a well-known example, is a polysaccharide containing mannuronic and glucuronic acid residues that readily forms ionically crosslinked hydrogels in the presence of divalent cations, particularly calcium ions⁹⁷. This process occurs under mild conditions, including room temperature and physiological pH, making alginate gels highly suitable for biomedical applications, such as cell encapsulation⁹⁸ and protein release systems⁹⁹. Notably, these gels can be selectively destabilized through the extraction of calcium ions using chelating agents, enabling controlled release applications.

Crosslinking via ionic interactions is not limited to anionic polymers. Chitosan, a polycationic biopolymer derived from the deacetylation of chitin, consists of β-(1-4)-linked glucosamine units and can form hydrogels upon crosslinking with glycerol-phosphate disodium salt. Remarkably, chitosan solutions remain liquid at lower temperatures but undergo rapid gelation upon heating, a behavior influenced by the degree of deacetylation of the polymer. This thermosensitive property allows chitosan-based systems to gel upon reaching physiological temperatures, making them suitable for in vivo applications such as protein or chondrocyte delivery, as demonstrated by histological studies showing effective protein-induced bone and cartilage formation¹⁰⁰.

Interestingly, polymers without typical ionic binding sites, such as dextran, can also form hydrogels in the presence of certain cations. Watanabe et al elucidated that dextran forms a hydrogel in the presence of potassium ions, with the potassium ions fitting precisely into a structural cavity created by six oxygen atoms from glucose units across three dextran chains¹⁰¹. Additionally, hydrogels can result from complexation between polyanions and polycations, such as chitosan with polyanions like dextran sulfate or polyphosphoric acid, yielding ionically crosslinked chitosan hydrogels. These hydrogels have demonstrated capability in drug delivery applications, exemplified by the encapsulation of doxorubicin, where nanoparticle formation allowed for minimal burst release while maintaining in vitro cytotoxicity¹⁰².

Freeze-thawing represents another prominent physical cross-linking technique in hydrogel formation. Initial developments in freeze-thawed hydrogels were pioneered by Peppas in 1975¹⁰³, who demonstrated that PVA solutions undergo gelation through repeated freeze-thaw cycles, driven by phase separation and crystallization processes^{104, 105}. This approach has since been extensively explored, leading to a range of studies by Peppas and others, further advancing the understanding and application of freeze-thawed hydrogels. A defining characteristic of this class of hydrogels is the capacity for facile customization of properties, coupled with ultra-purity, biocompatibility, and enhanced mechanical strength. These attributes make physically crosslinked hydrogels particularly valuable for applications in biomedicine and related fields, where high purity and gentle gelation conditions are essential.

Chemical crosslinking of hydrogels is a crucial technique in developing stable, structurally supportive hydrogel networks suitable for biomedical applications. Hydrophilic polymers containing hydroxyl (—OH) groups, such as PVA, can be crosslinked using glutaraldehyde as a crosslinker under controlled, stringent conditions involving low pH, the addition of methanol as a quenching agent, and elevated temperatures¹⁰⁶. Alternatively, polymers bearing amine groups, such as gelatin¹⁰⁷ and albumin¹⁰⁸, or amine-containing polysaccharides¹⁰⁹, can be crosslinked with glutaraldehyde under milder conditions, resulting in the formation of Schiff bases. This approach is particularly beneficial in synthesizing crosslinked proteins, where controlled and selective crosslinking is essential.

Photo-crosslinking, a specialized chemical crosslinking method, is frequently used to preserve the structural integrity of hydrogels in fabricated constructs by minimizing structural collapse. Chemical crosslinking mechanisms rely on covalent bond formation through chemical reactions, a process typically induced by UV irradiation, dehydrothermal treatment, or crosslinker addition. In photo-crosslinking, hydrogels are synthesized by exposing a photosensitive system, composed of unsaturated prepolymers and photoinitiators, to ultraviolet or visible light. This technique has gained attention for synthesizing biocompatible hydrogels, particularly through two main pathways: free-radical-initiated chain polymerization and bio-orthogonal click reactions. Though primarily chemical, certain physically crosslinked networks, such as thermal gelation in collagen¹¹⁰ and ultrasonic crosslinking in silk fibroin¹¹¹, may coexist within photo-crosslinkable hydrogels.

Free-radical polymerization, another method of producing chemically crosslinked hydrogels, involves the polymerization of hydrophilic polymers derivatized with polymerizable groups or mixtures of vinyl monomers. Natural, synthetic, and semi-synthetic hydrophilic polymers have been employed in this approach, with enzymes often acting as catalysts to introduce methacrylic groups into mono- and disaccharides, facilitating hydrogel synthesis¹¹¹. UV-polymerization further enables tailored hydrogel synthesis, supporting the development of complex and photo-reversible systems. This reversibility allows preformed hydrogels to degrade under UV light, enabling drug release¹¹², making this method particularly advantageous for therapeutic applications requiring controlled release. Thus, chemical crosslinking methods, including both free-radical and photo-crosslinking approaches, provide robust and versatile routes for hydrogel synthesis. These methods support applications across drug delivery, tissue engineering, and biomedical device fabrication by enabling structural integrity, biocompatibility, and tailored degradation profiles in hydrogel constructs.

The inherent optical scattering and absorption properties of biological tissues significantly limit light penetration depth, which is typically constrained to 50-100 μm for blue light (400-500 nm) and 1-3 mm for red and near-infrared light (650-1000 nm) under the skin¹¹³. To overcome these limitations, optical fibers can be embedded within the body to deliver light to, or collect optical signals from, deeper tissue regions. Traditional optical fibers, constructed from glass and plastic, are limited by their rigidity and static nature, making them unsuitable for integration with the natural micro- and macro-motions of biological tissues¹¹⁴. In contrast, hydrogels, with their high transparency, low modulus, and favorable biocompatibility, offer a promising alternative for developing flexible, biocompatible optical fibers. These hydrogel-based fibers enable bidirectional, real-time light transmission between internal organs and external optical systems¹¹⁵, and have been recently deployed in the nervous system for optogenetic modulation with potential applications in treating neuropsychiatric disorders¹¹⁶. Furthermore, hydrogel fibers present viable pathways for laser-based surgical interventions (e.g., deep tissue ablation, retinal photocoagulation, and cardiovascular plaque removal) and light-activated therapies (e.g., photodynamic therapy)¹¹⁷.

A notable advancement in this field was made by Choi et al., who developed hydrogel optical fibers using highly crosslinked polyethylene glycol (PEG) hydrogels for in vivo optical sensing and therapeutic applications¹¹⁸. These PEG-based fibers demonstrated high biocompatibility and excellent light-guiding properties but were limited by mechanical brittleness and a lack of stretchability. To address these limitations, Guo et al. fabricated hydrogel optical fibers using a highly stretchable and resilient polyacrylamide-alginate (PAAm-alginate) hydrogel, which overcame the brittleness and low flexibility issues of PEG-based fibers. These improved hydrogel fibers, optimized for guiding efficiency, mechanical stability, and softness, are suitable for light transmission across organ-scale distances and can adapt to dynamic and delicate tissues, such as the brain and spinal cord¹¹⁹.

Despite recent advancements, hydrogel optical fibers face significant challenges in maintaining long-term optical and mechanical performance. The refractive index of fully hydrated hydrogels, typically similar to that of water (~1.333), reduces the potential for total internal reflection at the interface between the hydrogel and surrounding tissues, leading to considerable light leakage. Additionally, in dynamic biological environments, hydrogel fibers may experience deformation and curvature, resulting in further light loss. To mitigate these challenges, various strategies have been implemented, including designing hydrogel fibers with core-cladding structures¹²⁰, engineering hydrogels with high refractive index nanodomains or nanofillers, refining fabrication techniques to produce smooth hydrogel surfaces, and developing anti-fatigue hydrogels with enhanced mechanical robustness⁹⁵.

A carbon nanotube (CNT) is a tube made of carbon with a diameter in the nanometer range (nanoscale). They are one of the allotropes of carbon. Two broad classes of carbon nanotubes are recognized: Single-walled carbon nanotubes (SWCNTs) have diameters around 0.5-2.0 nm, and Multi-walled carbon nanotubes (MWCNTs) consist of nested single-wall carbon nanotubes in a nested, tube-in-tube structure. Double- and triple-walled carbon nanotubes are special cases of MWCNT.

Carbon nanotubes can exhibit remarkable properties, such as exceptional tensile strength and thermal conductivity because of their nanostructure and strength of the bonds between carbon atoms. Some SWCNT structures exhibit high electrical conductivity while others are semiconductors. In addition, carbon nanotubes can be chemically modified. Carbon nanotubes are either metallic or semiconducting along the tubular axis.

Carbon nanotubes can be functionalized to attain desired properties that can be used in a wide variety of applications. The two main methods of carbon nanotube functionalization are covalent and non-covalent modifications. Because of their apparent hydrophobic nature, carbon nanotubes tend to agglomerate hindering their dispersion in solvents or viscous polymer melts. The resulting nanotube bundles or aggregates reduce the mechanical performance of the final composite. The surface of the carbon nanotubes can be modified to reduce the hydrophobicity and improve interfacial adhesion to a bulk polymer through chemical attachment. Chemical routes such as covalent functionalization have been studied extensively, which involves the oxidation of CNTs via strong acids (e.g. sulfuric acid, nitric acid, or a mixture of both) in order to set the carboxylic groups onto the surface of the CNTs as the final product or for further modification by esterification or amination. Free radical grafting is a promising technique among covalent functionalization methods, in which alkyl or aryl peroxides, substituted anilines, and diazonium salts are used as the starting agents. Functionalization can improve CNTs characteristically weak dispersibility in many solvents, such as water—a consequence of their strong intermolecular p-p interactions. This can enhance the processing and manipulation of insoluble CNTs, rendering them useful for synthesizing innovative CNT nanofluids with impressive properties that are tunable for a wide range of applications. Free radical grafting of macromolecules (as the functional group) onto the surface of CNTs can improve the solubility of CNTs compared to common acid treatments which involve the attachment of small molecules such as hydroxyl onto the surface of CNTs. The solubility of CNTs can be improved significantly by free-radical grafting because the large functional molecules facilitate the dispersion of CNTs in a variety of solvents even at a low degree of functionalization. Recently an innovative environmentally friendly approach has been developed for the covalent functionalization of multi-walled carbon nanotubes (MWCNTs) using clove buds. This approach is innovative and green because it does not use toxic and hazardous acids which are typically used in common carbon nanomaterial functionalization procedures. The MWCNTs are functionalized in one pot using a free radical grafting reaction. The clove-functionalized MWCNTs are then dispersed in water producing a highly stable multi-walled carbon nanotube aqueous suspension (nanofluids). Conductive nanorods may be formed from metals, ceramics or doped ceramics, or conductive polymers. See, en.wikipedia.org/wiki/Carbon_nanotube; U.S. Patents and Pub. patent application No. 20180366671; US20180340204; 20180210313; 20180134879; 20170097705; 20150188106; 20150064675; 20150044656; 20140357013; 20140272870; 20140197398; 20130341028; 20130213140; 20130218050; 20130187274; 20120257854; 20120155801; 20120090056; 20110025577; 20100267883; 20100264033; 20100261343; 20100187502; 20100139946; 20100013365; 20080315229; 20070267735; 20070176255; 20070132046; 20070127285; 20070085156; 20070029561; 20060043598; 20050075708; 20050035344; 20050035367; 20050036365; 20050035787; 6798000; 20030142456; 6519221; 20030001091; 20020110177; 20020109082; 6322713.

Overall, the continued development of hydrogel photonic devices promises to advance the integration of biocompatible, flexible optical systems for in vivo applications, offering transformative capabilities in biomedical imaging, sensing, and therapeutic interventions.

Due to their similar mechanical characteristics and versatility in biofunctional, mechanical, and electrical modifications, hydrogels have garnered increasing interest as materials for bioelectronics, offering potential for improved integration between biological and electronic systems. Hydrogels are typically electrical insulators, as they lack mobile charge carriers, resulting in conductivity levels under physiological conditions that are much lower than those of conventional electronic materials, like metals. To address this limitation, several strategies have been developed to enhance the electrical conductivity of hydrogels, including (a) incorporating ionic salts to produce ionically conductive hydrogels¹²¹⁻¹²³, (b) embedding electrically conductive micro- and nano-materials to introduce electronic conductivity¹²⁴, and (c) integrating conducting polymers into hydrogels to facilitate electronic conduction^{125, 126}.

Ionically Conductive Hydrogels: Ionically conductive hydrogels achieve conductivity through the movement of dissolved ionic salts (e.g., Na⁺, Li⁺, and Cl⁻) within the hydrogel matrix under an applied electric field. These hydrogels can maintain their inherent mechanical compliance and optical transparency while demonstrating ionic conductivities of up to ~10 S m⁻¹ at ~3 M salt concentration¹²⁴. Applications of ionically conductive hydrogels in electronic devices include diodes¹²⁷, sensors, actuators, signal transmitters, memristors, photovoltaic cells, and power sources¹²⁸. For instance, power sources have been developed by stacking hydrogel layers with varying salt concentrations and ionic selectivity, generating ~150 mV in open-circuit voltage¹²⁹. Despite their promise, ionically conductive hydrogels face challenges regarding biocompatibility; their salt concentrations are often significantly higher than physiological levels (~0.15 M), which can disrupt tissue homeostasis and reduce conductivity as salts diffuse from the hydrogel into the extracellular environment. Consequently, these hydrogels are best suited for non-invasive applications or devices that do not directly contact bodily fluids.

Conductive Micro- and Nanocomposite Hydrogels: Embedding conductive fillers, such as carbon-based or metallic nanoparticles, into hydrogels can endow them with electronic conductivity. Fillers like silver nanowires¹³⁰, gold nanoparticles¹³¹, carbon nanotubes¹³¹, graphene¹³², and liquid metals¹³³ have been incorporated to create hydrogel composites that retain mechanical flexibility while achieving electrical conductivity. These composites enable diverse applications, including underwater acoustic detection with silver nanowire-loaded PAAm hydrogels, cardiac patches with gold nanowires in alginate hydrogels or carbon nanotubes in gelatin methacrylate, and bio-integrated electrodes for electromyography (EMG) and ECG recording using graphene-based hydrogels. A key challenge in these composite hydrogels lies in balancing electrical and mechanical properties; achieving high conductivity requires a filler concentration above the percolation threshold¹³⁴⁻¹³⁷. However, excessive filler content can lead to aggregation, diminishing the composite's mechanical integrity.

Conducting Polymer Hydrogels: Conducting polymer hydrogels rely on high electron mobility along conjugated π-orbital chains, offering a pathway for enhanced electronic conductivity^{138, 139}. Polymers such as poly(3,4-ethylenedioxythiophene) (PEDOT)^{140, 141}, polypyrrole (PPy)¹⁴², and polyaniline (PANI)¹⁴³ have been shown to provide hydrogels with robust mechanical and electrical properties, high biocompatibility, and processability for chemical modifications¹⁴⁴. These conductive polymers can either form interpenetrating networks (IPNs) with non-conductive polymers or be developed as pure conducting polymer hydrogels. PEDOT hydrogels are particularly promising as interfaces for biological recording and stimulation due to their combined ionic and electronic conductivity, which results in low interfacial impedance.

In summary, advances in ionically conductive, composite, and conducting polymer hydrogels underscore the potential of hydrogels as viable, tunable materials for bioelectronic applications, providing promising opportunities for seamless integration with biological tissues in sensing, stimulation, and therapeutic technologies.

Hydrogel-based delivery systems have emerged as promising platforms for therapeutic drug delivery, providing clinically advantageous outcomes by enabling spatial and temporal control over the release of therapeutic agents, including small-molecule drugs, macromolecular drugs, and cells. Due to their tunable physical properties, controlled degradability, and ability to protect sensitive drugs from degradation, hydrogels facilitate diverse physiochemical interactions that modulate drug release. This review examines the multiscale mechanisms fundamental to the design of hydrogel drug delivery systems, with particular focus on the physical and chemical properties of hydrogel networks and the interactions between hydrogels and drugs at the network, mesh, and molecular levels. Further, these mechanisms was explored with respect to how they interact and integrate to achieve precise spatiotemporal control of drug presentation, while also providing quantitative analyses and clinical translation insights to inform the rational design of hydrogel delivery systems.

Traditional drug administration often requires high dosages or repeated delivery to achieve therapeutic effects, which can compromise efficacy, reduce patient compliance, and cause significant side effects or toxicity¹⁴⁵. For instance, systemic administration of interleukin-12 (IL-12) has led to severe toxicities, even fatal, in clinical trials¹⁴⁶. Oral administration, the most common route, often suffers from limitations in targeting efficacy and short circulation times, typically under 12 hours¹⁴⁷. Peptide and protein-based drugs further complicate administration due to their short serum half-lives, which may range from mere minutes to a few hours¹⁴⁸. To address these issues, controlled drug delivery systems have been developed, including membranes, nanoparticles, liposomes, and hydrogels. These systems offer precise control over drug availability to cells and tissues in both time and space, thereby potentially enhancing therapeutic efficacy and reducing toxicity and required dosage.

Hydrogels differ in size, architecture and function, and together these features dictate how hydrogels are used for drug delivery. In hydrogels, there are features with length scales spanning from centimetres to sub-nanometres. The macroscopic design largely determines the routes by which hydrogels can be delivered into the human body. Hydrogels can be formed into almost any overall size and shape. Micropores, if present, dramatically affect the overall physical properties (for example, the deformability), while allowing for convective drug transport. On the several-nanometre scale, a crosslinked polymeric network surrounds the water contained in the hydrogel network. Such networks contain open spaces, the size of which is referred to as the mesh size of the network. Importantly, the mesh size governs how drugs diffuse inside the hydrogel network. Finally, at the molecular and atomistic scale, various chemical interactions may occur between the drugs and the polymer chains. The polymer chains can possess numerous sites for binding interactions with the drugs, and these can be pre-designed using diverse physical and chemical strategies. The features at the mesh scale and the molecular and atomistic scale are essential for controlled drug release. Because they are decoupled from the macroscopic properties of the hydrogel, desirable features at each length scale can often be designed independently of the other. This multiscale nature facilitates the modular design of hydrogels, which can serve as a versatile platform to meet specific application-based requirements.

Neuronal stimulation by light represents a cutting-edge approach in the field of optogenetics, where genetic engineering is employed to introduce light-sensitive ion channels, such as microbial opsins, into specific neuronal population. This technique enables precise spatiotemporal control over cellular activity, facilitating the dissection of neural circuits and functional pathways within the nervous system^{32, 149}. Optogenetic stimulation is typically achieved by delivering light through fiber optics implanted at the targeted sites, activating or inhibiting genetically modified, photoactive cells in response to specific wavelengths of light¹⁵⁰. In this way, multiple channels, each responsive to distinct wavelengths, can be selectively activated within the same experiment, allowing for highly refined investigations of neuronal activity and interactions.

For efficient neuronal stimulation, precise and reliable light delivery is critical, particularly within the complex and delicate environment of the nervous system. Conventional polymer-based optical fibers, including those made from polycarbonate¹⁵¹, polymethyl methacrylate¹⁵² polystyrene¹⁵³, and cyclic olefin copolymer⁶⁴, have enabled optogenetic applications in various anatomical regions such as the gut lumen and spinal cord. However, the rigidity of traditional optic materials, with moduli in the gigapascal (GPa) range, poses a significant challenge for applications in soft tissues (modulus in kPa to MPa ranges). These stiff materials risk damaging tissues under repeated deformation and restrict the natural behaviors of experimental subjects. Although microscale light-emitting diodes (μ-LEDs) implanted near target sites offer an alternative for dynamic neural stimulation, their application is hindered by the need for complex wireless power systems, precise control mechanisms, and intricate fabrication and implantation procedures¹⁵⁴.

Due to their high optical transparency within the visible spectrum and tunable mechanical properties, hydrogels have emerged as promising candidates for optical applications. Hydrogels have already demonstrated utility in expansion microscopy¹⁵⁵, where their swelling properties enhance image resolution, as well as in tissue clearing techniques that facilitate visualization of cellular structures^{93, 156}. However, their application as optical fibers have been limited by mechanical vulnerabilities. Conventional hydrogel materials suffer from fatigue-induced fracture under repeated deformations during animal locomotion, and the interface between rigid materials (e.g., silica) and soft hydrogels used for light coupling is prone to failure under mechanical stress. Additionally, the low refractive indices (RI) of hydrated hydrogels (~1.34) and insufficient RI contrast between core and cladding layers lead to substantial light leakage along the fiber length.

See, U.S. patent and Pub. patent App. Nos. U.S. Pat. Nos. 4,366,901; 4,692,273; 4,706,680; 4,777,954; 4,989,607; 5,143,071; 5,217,594; 5,225,064; 5,334,296; 5,374,241; 5,391,272; 5,405,366; 5,429,886; 5,468,366; 5,505,715; 5,534,132; 5,607,691; 5,622,168; 5,622,944; 5,735,273; 5,746,207; 5,800,685; 5,972,199; 5,993,219; 6,021,348; 6,139,718; 6,205,359; 6,208,902; 6,269,270; 6,281,006; 6,352,524; 6,366,814; 6,391,558; 6,482,299; 6,485,703; 6,527,716; 6,564,102; 6,611,715; 6,615,081; 6,629,968; 6,668,191; 6,767,747; 6,800,278; 7,062,330; 8,565,850; 9,586,038; 10,076,279; 10,938,032; 11,083,371; 11,116,448; 11,141,129; 11,141,589; 11,141,590; 11,199,903; 11,207,025; 11,395,630; 11,510,841; 11,806,521; 11,890,468; 12,135,286; 12,144,614; 12,245,859; 12,245,860; 20010002441; 20010003799; 20010017269; 20020001799; 20020012943; 20020019588; 20020026110; 20020095080; 20020113606; 20020117408; 20020165590; 20030055478; 20030078499; 20030083729; 20040038090; 20040087671; 20040243018; 20050004621; 20050019215; 20050131288; 20050131484; 20050136023; 20050136077; 20050137276; 20050154425; 20050154426; 20050165458; 20050187590; 20050192644; 20050197678; 20050209654; 20050216070; 20050228335; 20050228336; 20050251051; 20050251424; 20050277991; 20060009815; 20060079936; 20060142810; 20060178706; 20060184211; 20070005052; 20070047752; 20070059459; 20070060815; 20070067004; 20070078372; 20070078373; 20070093705; 20070093706; 20070093743; 20070111202; 20070196320; 20070202562; 20080029390; 20080033492; 20080207779; 20080210592; 20090005667; 20090132018; 20090177131; 20090326602; 20100016702; 20100016703; 20100016929; 20100036230; 20100062067; 20100076533; 20100114238; 20100114273; 20100160762; 20100256474; 20100318018; 20100324638; 20110021937; 20110087315; 20110112418; 20110125040; 20110208067; 20110208076; 20110230701; 20110230938; 20110257504; 20110263994; 20110276107; 20110276112; 20110305970; 20110306862; 20120029416; 20120029591; 20120029601; 20120041296; 20120058101; 20120078338; 20120101326; 20120101396; 20120184801; 20120185020; 20120190956; 20120203156; 20120258467; 20120322685; 20120330395; 20130053934; 20130066395; 20130102874; 20130131746; 20130146821; 20130214850; 20140029162; 20140045065; 20140051946; 20140200387; 20140206977; 20140257016; 20140296418; 20140296931; 20140296934; 20140316485; 20140318985; 20140336730; 20150018705; 20150025479; 20150057519; 20150088224; 20150094559; 20150165186; 20150238754; 20150248833; 20150265836; 20150289923; 20150297904; 20150335876; 20150335877; 20150377877; 20160004298; 20160023005; 20160025669; 20160081580; 20160101282; 20160101288; 20160120434; 20160123865; 20160169835; 20160228691; 20160230197; 20160242654; 20160244554; 20160289851; 20160291001; 20160346530; 20160361015; 20160377606; 20170015881; 20170021172; 20170056682; 20170080207; 20170087766; 20170101513; 20170157398; 20170157401; 20170165470; 20170181669; 20170209695; 20170224990; 20170238851; 20170239459; 20170251967; 20170258402; 20170299538; 20170303815; 20170309365; 20170312521; 20170316487; 20170332967; 20170342213; 20170350882; 20170356812; 20170367614; 20170368329; 20180000347; 20180001086; 20180014780; 20180015285; 20180078686; 20180078754; 20180085580; 20180093093; 20180111353; 20180116546; 20180125689; 20180132757; 20180132790; 20180133457; 20180136247; 20180161579; 20180177430; 20180177996; 20180198218; 20180281366; 20180294518; 20180318583; 20180323400; 20180333574; 20180345014; 20180368743; 20190000338; 20190004004; 20190015008; 20190022386; 20190022400; 20190046065; 20190083784; 20190105488; 20190117062; 20190117974; 20190122584; 20190134393; 20190170739; 20190217078; 20190237268; 20190254579; 20190269914; 20190313932; 20190321636; 20190329022; 20190374773; 20190381313; 20200000407; 20200024631; 20200032822; 20200038655; 20200038658; 20200040468; 20200053487; 20200061379; 20200085299; 20200094040; 20200138313; 20200138702; 20200179694; 20200187823; 20200188294; 20200188660; 20200191757; 20200197707; 20200205673; 20200238085; 20200253555; 20200257199; 20200308577; 20200324107; 20200325586; 20200338333; 20200338334; 20200353156; 20200376266; 20200377677; 20200406033; 20210003149; 20210008376; 20210023360; 20210047745; 20210052883; 20210087336; 20210100998; 20210113830; 20210121402; 20210122895; 20210137402; 20210137455; 20210140832; 20210142886; 20210178157; 20210187160; 20210187278; 20210213286; 20210220640; 20210228134; 20210236806; 20210252285; 20210259634; 20210268276; 20210268278; 20210290954; 20210299438; 20210309877; 20210315587; 20210330216; 20210330963; 20210330969; 20210338141; 20210338158; 20210343922; 20210353198; 20210361183; 20210402172; 20210402182; 20210403613; 20220000407; 20220003676; 20220016413; 20220039720; 20220039722; 20220040473; 20220040480; 20220054820; 20220062621; 20220088372; 20220118245; 20220143393; 20220160995; 20220192570; 20220199994; 20220233844; 20220233848; 20220238942; 20220257945; 20220273947; 20220274372; 20220288405; 20220296165; 20220296804; 20220305260; 20220355107; 20220409847; 20220409902; 20230019206; 20230034846; 20230047195; 20230066856; 20230121718; 20230149144; 20230149703; 20230149704; 20230158293; 20230190124; 20230191126; 20230200729; 20230211161; 20230218890; 20230240586; 20230284953; 20230309910; 20230320609; 20230329916; 20230365775; 20230372707; 20230381476; 20230381513; 20230381515; 20230397870; 20230414928; 20240017056; 20240033521; 20240049994; 20240050744; 20240066308; 20240090600; 20240091538; 20240100218; 20240100225; 20240100330; 20240189579; 20240198100; 20240216691; 20240237938; 20240263002; 20240269352; 20240278012; 20240316338; 20240321455; 20240325728; 20240332613; 20240341675; 20240350797; 20240350800; 20240366134; 20240374889; 20240390172; 20240415404; 20250000392; 20250017509; 20250018185; 20250018191; 20250025078; 20250025108; 20250034337; 20250057461; 20250059103; 20250064373; 20250065107; 20250072806; 20250082924; and 20250082932. See also: neuralink.com/and axoft.us.

### SUMMARY OF THE INVENTION

To address these limitations, multifunctional neural probes were developed integrating optical interrogation, electrical recording, and chemical intervention capabilities using soft, biocompatible hydrogel materials. Polyvinyl alcohol hydrogels were selected due to their high transparency in the visible range, fatigue resistance, and biocompatibility. The PVA hydrogel fibers were fabricated through chemical cross-linking, resulting in fibers with high stretchability (139.3-169.2%), low elastic moduli (2.8-9.3 MPa), and low bending stiffness (4.6±1.4 N/m). By optimizing the optical properties of these hydrogel fibers, including light transmission and refractive index, high refractive indices (1.37-1.40 at 480 nm) and excellent light transmission (>96%) were achieved. This enabled the fabrication of step-index optical fiber hydrogel probes with a high refractive index contrast, facilitating in vivo optical recordings and optogenetic stimulation in mice. The application of these hydrogel optical probes in the ventral tegmental area of mice, combined with fiber photometry and social behavioral assays, demonstrated their effectiveness.

Simultaneously, high-aspect-ratio conductive nanofillers were incorporated into the PVA hydrogel matrix to create conductive hydrogel microelectrodes. These microelectrodes exhibited high fatigue resistance (20,000 cycles at 20% strain), stretchability (64.5±7.9%), and low electrochemical impedance (33.20±9.27 kΩ@ 1 kHz in 1 cm length). These properties enabled reliable electrophysiological recordings and monitoring of spontaneous neural activities in both deep brain regions and the spinal cord of mice. To enable chemical interventions, ice templating and freeze-drying techniques were applied to the hydrogel fibers, creating microscale canals with controllable patterns (30-75%) across the fiber cross-section. These micro-patterned fibers facilitated in vivo delivery of adeno-associated viruses in mice. This work represented an advancement in the integration and miniaturization of optical waveguides and microelectrodes into a single, hydrogel-based device. The resulting multifunctional neural probes allowed for simultaneous optogenetic stimulation and electrophysiological recordings of light-evoked neural activities in Channelrhodopsin-2 transgenic mice.

Creating durable, motion-compliant neural interfaces is crucial for accessing dynamic tissues under in vivo conditions and linking neural activity with behaviors. Utilizing the self-alignment of nano-fillers in a polymeric matrix under repetitive tension. Conductive carbon nanotubes may be introduced with high aspect ratios into semi-crystalline polyvinyl alcohol hydrogels and create electrically anisotropic percolation pathways through cyclic stretching. The resulting anisotropic hydrogel fibers (diameter of 187±13 μm) exhibit fatigue resistance (20,000 cycles at 20% strain) with a stretchability of 64.5±7.9%, and low electrochemical impedance (900 #149 kΩ@ 1 kHz). The re-constructed nanofillers' axial alignment and a corresponding anisotropic impedance decrease is observed along the direction of cyclic stretching. Fiber-shaped hydrogels are fabricated into bioelectronic devices and implant them into wild-type and transgenic Thy1-ChR2-EYFP mice to record electromyographic signals from muscles in anesthetized and freely moving conditions. These hydrogel fibers effectively enable the simultaneous recording of electrical signals from ventral spinal cord neurons and the tibialis anterior muscles during optogenetic stimulation. Importantly, the devices maintain functionality with repeatable recording results over eight months after implantation, demonstrating their durability and potential for long-term monitoring in neurophysiological studies.

To address these challenges, PVA was selected to develop a hydrogel matrix with enhanced fatigue resistance due to its semi-crystalline structure^{95, 150, 157, 158}, which is conducive to long-term mechanical stability in optical fiber applications. Through controlled crystallization and chemical cross-linking, the optical properties of PVA hydrogels—including RI, transmittance, and attenuation—were fine-tuned to optimize light coupling efficiency. Using molding and extrusion techniques, step-index hydrogel optical fibers were fabricated with a high-RI core and a thin cladding layer to create a substantial RI contrast, minimizing light leakage and facilitating robust light delivery for in vivo optogenetic applications.

A soft, stretchable microelectrode composed of a semi-crystalline hydrogel with a chemically crosslinked polyvinyl alcohol matrix, enhanced by nanoscale conductive fillers, specifically carbon nanotubes, is provided according to one embodiment. By utilizing Tension Reinforced Anisotropic Nano-orientation (TRAIN) strategies, anisotropic conductivity is p-provided with a tunable elastic modulus of the electrode. The inclusion of nanofillers, capable of realigning under mechanical stress, enhances the conductivity of the microelectrodes. Moreover, the semi-crystalline hydrogels combined with high-aspect-ratio nanofillers enhance fatigue resistance and maintain conductivity under repetitive and complex mechanical conditions, such as bending and stretching. This technology has been successfully applied to collect electrical signals from the muscles and spinal cords of freely moving rodents.

Hydrogel microelectrodes to offer a minimally invasive method for recording electrical signals from muscles, spinal cords, and other regions involved in motion. These microelectrodes are soft, stretchable, and hydrated, allowing them to integrate seamlessly into tissues during movement without causing significant dislocations within the tissues. They may be designed for durability and to maintain stability in both mechanical and electrical properties over several months in vivo.

The technology may provide a recording solution for spinal cord injuries. According to this embodiment, natural movement is monitored without constraining the subject, avoiding pain, or inflicting further tissue damage. This positions the technology as a valuable tool in both medical research and therapeutic applications, addressing a critical need in the treatment and understanding of spinal cord injuries and other movement-related disorders.

PVA hydrogels were designed with embedded with carbon nanotubes (CNTs). Cyclic mechanical stretching was applied to PVA-CNT hydrogel fibers to regulate their impedance. Anisotropic conductivity improvement aligned with the stretching direction after cyclic stretching (5%, 10% or 20% strain, 5,000 cycles) was observed. X-ray, electron microscopy and stimulated Raman spectrum characterization also revealed anisotropic nanocrystalline alignment. These PVA-CNT hydrogel fibers maintained 56.6% stretchability and 900±149 kΩ (@ 1 kHz) with a diameter of 205.8±20.9 μm.

When the PVA-CNT fibers were fabricated into hydrogel electrodes, light-evoked muscle response and electromyographic (EMG) signals were successfully detected upon blue light (473 nm) transdermal stimulation in Thy1::ChR2-EYFP transgenic mice. The Thy1-ChR2 mice express blue-light-sensitive ion channels throughout their nervous systems. Non-invasive transdermal optogenetics stimulation trigger neural excitation and the hydrogel electrodes effectively record the EMG signals in both anesthetized and freely moving mice.

To further evaluate the applicability of the PVA-CNT hydrogel electrodes for studying nerve-muscle signal transmission, hydrogel electrodes (138.2±8.5 μm) were implanted into the ventral horn of the spinal cord and the hindlimb muscles of Thy1-ChR2 mice. Light-evoked electrical signals in the mouse spinal cord and the EMG were simultaneously detected, which showed a strong correlation with pulsed transdermal light stimulation. By linking the transdermal optogenetic stimulation, spinal cord electrical recording, and EMG recording, the utility of hydrogel electrodes for peripheral nerve-spinal cord-muscle circuits was demonstrated.

The electrodes are not limited to central nervous system application, and may be used in any tissue, especially electrically excitable tissues such as smooth muscle, skeletal muscle, cardiac muscle, peripheral nerves, intestine, glands, and the like.

The electrodes may also be used in spinal cord modulating devices for spinal cord injury patients' recovery and brain recording devices.

The microelectrodes are more affordable to produce that micromachined electrodes, since they eliminate the need for expensive lithography technology and sophisticated manufacturing facilities. This reduction in production costs makes them a more accessible option.

These microelectrodes exhibit superior functional longevity once implanted. They are designed to maintain their mechanical and electrical properties over extended periods in vivo, which is crucial for long-term monitoring and treatment applications without the need for frequent replacements.

The materials and design of the hydrogel microelectrodes ensure higher biocompatibility compared to traditional electrodes. This means they are less likely to cause adverse reactions when implanted, making them safer for long-term use in patients.

Unlike rigid electrodes, the hydrogel microelectrodes can adapt to the natural motion of tissues. This flexibility minimizes the risk of causing damage or dislocations within the tissues, ensuring more reliable data collection and reducing the discomfort or potential complications for the subject.

It is therefore an object to provide a hydrogel with a chemically crosslinked polymer matrix, having conductive nanorods or nanotubes.

It is also an object to provide a hydrogel electrode for electrically interfacing with animal tissue, comprising with a chemically crosslinked polymer matrix configured having a diameter of less than 500 μm, having anisotropically aligned conductive nanorods or nanotubes, surrounded by a tubular insulating layer.

The hydrogel may be semi-crystalline.

The crosslinked polymer matrix may comprise polyvinyl alcohol, poly(3,4-ethylenedioxythiophene), polystyrene sulfonate (PEDOT:PSS), Polydimethylsiloxane (PDMS), Styrene-Ethylene-Butylene-Styrene (SEBS), Poly(1,8-octanediol-co-citric acid) (POC), Polyimide (PI), Polycarbonate (PC) and Cyclic olefin copolymer (COC), Poly(etherimide) (PEI), epoxy-based negative photoresist, and/or Poly(glycolic acid) (PGA).

The conductive nanorods or nanotubes may be anisotropically aligned.

The hydrogel may be configured as an elongated structure, and wherein the conductive nanorods or nanotubes are axially aligned with the elongated structure.

The chemically crosslinked polymer matrix may be soft and stretchable. For example, it may emulate various mechanical properties of a tissue in which it is to be inserted, e.g., cerebral issue, spinal cord tissue, etc.

The hydrogel may display anisotropic conductivity. The anisotropic conductivity may result from cyclic stress of the polymer matrix having the conductive nanorods or nanotubes, or cyclic strain of the polymer matrix having the conductive nanorods or nanotubes or Tension Reinforced Anisotropic Nano-orientation (TRAIN).

The conductive nanorods or nanotubes may be aligned by cyclic mechanical stretching of between 5% and 20% strain, over 5,000 cycles at about 0.5 Hz.

The crosslinked polymer matrix may have an elastic modulus of between 2.8 to 9.3 MPa, e.g., between 3 to 7 MPa, or 4 to 6 MPa. The elastic modulus is preferably below 5 MPa.

The crosslinked polymer matrix preferably has a stretchability of at least 50%, e.g., at least 65%, 75%, 85%, 90%, 100%, 125%, 139%, 140%, 150%, or 168%.

The crosslinked polymer matrix preferably has a bending stiffness of less than 6 N/m, e.g., 5 N/m, 4.6 N/m.

The polymer matrix may comprise Poly(vinyl) alcohol crosslinked with Glutaraldehyde.

The polymer matrix may be formed from poly(vinyl) alcohol having a molecular weight from 146,000 to 186,000 Da and which is at least 99% hydrolyzed.

The hydrogel may be configured as a rod having a diameter of less than 500 μm.

The hydrogel may have an outer coating of silicon dioxide, silica, aluminum oxide, styrene-ethylene-butylene-styrene (SEBS), reduced graphene oxide. The coating may be formed by dip coating, or atomic layer deposition, for example.

The hydrogel may have an inner tubular conduit to provide a path for fluid flow.

The hydrogel may have an inner fiber optic to provide optical communication.

The hydrogel may exhibit ionic conductivity, e.g., in addition to conduction through the conductive nanorods or nanotubes.

The hydrogel may provide a plurality of conductive paths, through a plurality of hydrogel wires or electrode, each configured as a rod having a diameter of less than 500 μm, 400 μm, 300 umm 250 μm, 225 μm, 200 μm, 180 μm, 175 μm, 150 μm, 125 μm, 110 μm, 100 μm, 90 μm, 80 μm, or 75 μm wherein each rod is electrically insulated from each other rod.

It is a further object to provide a method of making a soft, elastic, conductive hydrogel electrode, comprising: combining a hydrogel-forming polymer with carbon nanotubes and a carbon nanotube dispersing agent to form a heterogeneous mixture; crosslinking the hydrogel-forming polymer in a mold to form an elongated hydrogel having a diameter less than 1 mm; demolding the elongated hydrogel; and applying a cyclic strain on the molded hydrogel to align the carbon nanotubes with an elongation axis of the molded hydrogel to form an anisotropically-conductive hydrogel.

The anisotropically-conductive hydrogel may be coated with an insulating layer, a conductive layer, an optically reflective layer or a protective layer.

The method may further comprise drying or freeze-drying the anisotropically-conductive hydrogel.

The mold may be a silicone mold.

The method may further comprise acidifying the demolded elongated hydrogel to promote interaction between hydrogel-forming polymer chains.

The anisotropically-conductive hydrogel may be inserted into an animal tissue, and the anisotropically-conductive hydrogel reswelled with tissue fluid.

The method may further comprise annealing the dried anisotropically-conductive hydrogel.

The method may further comprise reswelling the annealed anisotropically-conductive hydrogel in an aqueous solvent.

The anisotropically-conductive hydrogel may be inserted into an animal tissue and electrical signals from the animal tissue recorded.

The anisotropically-conductive hydrogel may be inserted into an animal tissue, and an electrical current in the anisotropically-conductive hydrogel may be used to convey ions from the anisotropically-conductive hydrogel into the animal tissue, similar to iontophoresis.

An optical signal may be communicated through anisotropically-conductive hydrogel, e.g., to stimulate tissue at an end of the hydrogel, or to receive an optical emission from the tissue.

The anisotropically-conductive hydrogel may be coated with a coating configured to provide total internal reflection of an optical signal within the anisotropically-conductive hydrogel.

The hydrogel-forming polymer may be mixed with tetraethyl orthosilicate to modify a refractive index of the anisotropically-conductive hydrogel.

The anisotropically-conductive hydrogel may be coated with reduced graphine oxide, silica, alumina, or styrene-ethylene-butylene-styrene.

### BRIEF DESCRIPTION OF THE DRAWINGS

FIGS. 1A-1H show the TRAIN strategy for enhancing conductivity in hydrogel fibers. a, Schematic of the in vivo application of TRAIN microelectrodes, illustrating their implantation into muscle tissue and spinal cord with correlated electrical signals corresponding to muscle and the spinal cord. The TRAIN microelectrode was achieved by the alignment of carbon nanotube (CNTs) bundles in polyvinyl alcohol (PVA) hydrogel through cyclic stretching. b, Schematics of microstructures in PVA hydrogels under different phases: chemical cross-linking, nanocrystalline growth, cyclic training after incorporation of CNTs in PVA, and realignment of CNTs in PVA hydrogel matrix. c, Representative SEM images at the cross-section of untrained PVA hydrogel film and TRAIN CNTs (0.24 wt %)-PVA hydrogel film. Scale bars: 2 μm. d, Specific impedance of CNTs-PVA hydrogel fibers with different CNTs concentrations (n=5 individual CNTs-PVA hydrogel fibers). One-way ANOVA was used to determine statistical significance: F_{3,16}=34.75, ****p<0.0001. TEM images display CNTs at different concentrations. Scale bars: 50 nm. e, Comparative normalized impedance analysis of CNTs-PVA hydrogels with different CNTs concentrations across varied conditions (One-way ANOVA, 0.04 wt % CNTs: F_{2,11}=0.0799, non-significant (n.s.). 0.08 wt % CNTs: F_{2,11}=12.07, **p=0.0017. 0.16 wt % CNTs: F_{2,11}=11.91, **p=0.0018. 0.24 wt % CNTs: F_{2,11}=49.35, ****p<0.0001. n=4 individual CNTs-PVA hydrogel fibers and n=5 individual TRAIN hydrogel fibers). f, Normalized impedance profiles of CNTs-PVA (0.16 wt %) hydrogel fibers subjected to various cyclic training conditions (5%, 10%, and 20% strain for 5,000 cycles. One-way ANOVA, 5% training: F_{2,11}=10.94, **p=0.0024; 10% training: F_{2,11}=11.91, **p=0.0018; 20% training: F_{2,11}=6.342, *p=0.0147. n=4 individual CNTs-PVA hydrogel fibers and n=5 individual TRAIN hydrogel fibers). g, Impedance ratio (pre-training/post-10% training) of CNTs-PVA hydrogels with different CNTs concentrations (One-way ANOVA, F_{3,16}=5.798, **p=0.0070, n=5 individual TRAIN hydrogel fibers). Inset: schematic of CNTs-PVA hydrogel fibers undergoing cyclic training in water. All the data are presented as mean±standard deviation (s.d.). h, Normalized specific impedance of CNTs-PVA hydro microelectrodes under 20% strain training with different cycles.

FIGS. 2A-2L show mechanisms of TRAIN strategy. a, Illustration of the Stimulated Raman Scattering (SRS) microscopy setup, highlighting the path of the laser, the sample interaction, and signal detection components. b, Comparative two-color SRS images of untrained CNTs (0.16 wt %)-PVA hydrogel and TRAIN CNTs (0.24 wt %)-PVA hydrogel. Scale bars: 20 μm. c, Raman spectra displaying the characteristic G-band of CNTs within the TRAIN CNT (0.16 wt %)-PVA hydrogel film compared to the PVA hydrogel film. d, Representative probability of CNTs orientations calculated from b. e, Dispersion degrees of untrained CNTs (0.16 wt %)-PVA hydrogel and TRAIN CNTs (0.24 wt %)-PVA hydrogel. Data are presented as mean±s.d., n=8 individual hydrogel samples for untrained group and n=12 individual hydrogel samples for TRAIN group. Two-tailed unpaired student's t-test was used to determined statistical analysis: F_{7,11}=9.794, t=9.216, df=18, ****p<0.0001. f, Conductivity mechanisms in TRAIN CNTs-PVA hydrogel film along the x, y, and z directions, respectively. g, Left: A schematic of electrochemical characterization of TRAIN CNTs-PVA hydrogel film in x and y directions and an image of the characterization device. Scale: 1 mm. Right: An illustration of electrochemical characterization of TRAIN CNTs-PVA hydrogel film in z direction and an image of characterization device. Scale: 1 mm. WE: working electrode, RE: reference electrode, CE: counter electrode. h, Specific impedance changes (post-training/pre-training) of TRAIN CNTs-PVA hydrogels in x, y, z direction. i, Wide angle x-ray spectroscopy (WAXS) patterns of pre and post 20% trained CNTs-PVA hydrogel fibers. The break at 180° indicates the beam splits from the instrument. j, Elastic modulus of untrained CNTs (0.16 wt %)-PVA hydrogel fibers and TRAIN CNTs (0.16 wt %)-PVA hydrogel fibers. Data are presented as mean±s.d., n=8 individual hydrogel fibers. One-way ANOVA was used to determined statistical analysis: F_{3,28}=15.36, ****p<0.0001. k, Maximum elongation of untrained CNTs (0.16 wt %)-PVA hydrogel fibers and TRAIN CNTs (0.16 wt %)-PVA hydrogel fibers. Data are presented as mean±s.d., n=8 individual hydrogel fibers. One-way ANOVA was used to determined statistical analysis: F_{3,28}=9.364, ***p=0.0002. 1, Differential scanning calorimetry (DSC) curve of PVA and CNTs-PVA (0.16 wt % and 0.24 wt %) materials.

FIGS. 3A-3K show the TRAIN-enabled freely moving Electromyographic (EMG) recordings. a, Image of a TRAIN microelectrode in water, being manipulated by a single human hair to demonstrate its delicate structure and flexibility. Scale: 5 mm. b, Schematic of TRAIN microelectrode implantation within the tibialis anterior (TA) and gastrocnemius (GS) muscles of a mouse. C, Representative visualization of a TRAIN microelectrode inserted into the GS muscle. d, Confocal image of sciatic nerve cross-sections (coronal and longitudinal) showing Thy1-ChR2-EYFP expression. Scale bars: 50 μm. e, A schematic of hindlimb kinematics of a Thy1-ChR2-EYFP mouse with and without optical stimulation. f-g, EMG recordings from the TA and GS muscles from a Thy1-ChR2-EYFP mouse with overlaid electrical signals corresponding to induced muscle contractions. Shadows present as mean±s.d., blue bars indicate illumination cycles from a 473 nm laser. h, Pose estimation showing stance and swing phases from a mouse implanted with TRAIN microelectrodes in the TA and GS muscle during locomotion on a running wheel. i, Comparative gait analysis using pose estimation between a control mouse (without implant) and an experimental mouse (with implant). j, Quantitative evaluation of running distance, hindlimb retraction height, and duration for both non-implanted and implanted mice. Data are presented as mean±standard error of the mean (s.e.m.), n=7 individual running cycles for non-implanted mouse and n=6 individual running cycles for implanted mouse. k, A schematic showing a mouse on a running wheel, with a TRAIN hydrogel electrode implanted in the TA muscle to capture EMG signals during free locomotion. Examples of EMG recordings of the left TA and GS muscles from a mouse on a running wheel.

FIGS. 4A-4N show the Spinal cord electrophysiology with TRAIN microelectrodes. A, Image of an assembled microelectrode probe for implantation into the spinal cord. Scale: 5 mm. b, Top: Thy1-ChR2-EYFP mouse post-operative with an implanted probe targeting the Lumbar (L) 3 region of the spinal cord. Bottom: Confocal microscopy image of a transverse section of the spinal cord L3 region exhibiting Thy1-ChR2-EYFP expression. c, Schematic illustration of the integrated neuromodulation system in a Thy1-ChR2-EYFP mouse, depicting an electronic device implanted at the L3 segment of the spinal cord with concurrent hydrogel electrode implantation in the hindlimb muscle. This multimodal system comprises synchronized components: (i) optical neural stimulation, (ii) electrophysiological recording from the spinal cord, and (iii) EMG from the hindlimb muscle. d, Representative recordings of spinal cord electrophysiological and EMG activity elicited by transdermal optical stimulation in Thy1-ChR2-EYFP mice. Overlaid traces (n=10 individual events, mean±s.d.) under high- and low-intensity light stimulation illustrate the response dynamics. e, Endogenous spinal cord neural activity recorded from a mouse 1-month post-implantation under anesthesia. f, Overlaid wave forms of single neuron spiking from e and principal component analysis (PCA) from e showing a cluster of the overlaid waveforms. g, Spontaneous spinal cord neural activity recorded from an awake mouse 1-month post-implantation. h, Overlaid wave forms of single neuron spiking from g and PCA from g showing a cluster of the overlaid waveforms. i, Endogenous spinal cord neural activity recorded from a mouse 8 months post-implantation under anesthesia. j, Overlaid wave forms of single neuron spiking from i and PCA from i showing a cluster of the overlaid waveforms. k, Spontaneous spinal cord neural activity recorded from a conscious mouse 8 months after implantation 1, Overlaid single neuron spike waveforms from k, with PCA from k illustrating a cohesive cluster. m, A schematic and an image of a mouse with an electronic device implanted in the spinal cord L3. n, Spontaneous neural activity traces of a moving mouse from spinal cord L3 in an open field.

FIGS. 5A-5C show a, Images of a CNTs-PVA hydrogel fiber undergoing cyclic training (20% strain for 10000 cycles) in water. Scale bars: xx mm. b, Representative plots of force versus cycle number of a CNTs-PVA hydrogel fiber under cyclic tensile test. c, Zoomed in plot from cycle 5000 to 5010 in c.

FIGS. 6A and 6B show a, Water content fraction in TRAIN CNTs-PVA hydrogel microelectrodes after different cycles of training at 20% strain. b, Fiber diameter of TRAIN CNTs-PVA hydrogel microelectrodes after different cycles of training at 20% strain. One-way ANOVA was used to determine the statistical significance, Water content fraction: F_{5,18}=5.858, **p=0.0022, n=4 individual TRAIN hydrogel microelectrodes; Fiber diameter: F_{5,24}=0.7050, non-significant (n.s.), p=0.6253, n=5 individual TRAIN hydrogel fibers.

FIGS. 7A-7C show the normalized impedance of TRAIN hydrogel films measured in three directions: a, Direction along with the training direction on the training plane. b, Direction perpendicular to the training direction on the training plane. c, Direction along with the thickness of the TRAIN hydrogel films. Two-tailed paired student's t-test was used to determine statistical significance. X: t=3.807, df=5, α=0.05, *p=0.0125. Y: t=0.3202, df=5, α=0.05, non-significant (n.s.) p=0.7618. Z: t=5.846, df-5, α=0.05, **p=0.0021. n=6 independent hydrogel films.

FIGS. 8A-8D show the normalized dimensions of TRAIN hydrogel films: a, Direction along with the training direction on the training plane. b, Direction perpendicular to the training direction on the training plane. c, Direction along with the thickness of the TRAIN hydrogel films. Two-tailed paired student's t-test was used to determine statistical significance. d, Volume calculated from x, y, and z. x: t=3.510, df=5, α=0.05, *p=0.0171. y: t=3.873, df=5, α=0.05, *p=0.0117. z: t=3.088, df=5, α=0.05, *p=0.0272. volume: t=1.676, df=5, α=0.05, n.s. p=0.1546. n=6 independent hydrogel films.

FIGS. 9A-9D show the representative stress-strain curves of CNTs-PVA hydrogel fibers. a, Non-pre-stretched and untrained fiber. b, 200% pre-stretched and untrained fibers. c, 200% pre-stretched and TRAIN fibers (20% for 5000 cycles). d, 200% pre-stretched and TRAIN fibers (20% for 10000 cycles).

FIGS. 10A-10D show the representative linear regression (first 10% strain) for Young's modulus calculation of CNTs-PVA hydrogel fibers. a, Non-pre-stretched and untrained fiber. b, 200% pre-stretched and untrained fibers. c, 200% pre-stretched and TRAIN fibers (20% for 5000 cycles). d, 200% pre-stretched and TRAIN fibers (20% for 10000 cycles).

FIG. 11A-11D show the design and fabrication of PVA hydrogel fiber optic device. a, Schematic of the dip-coating process for creating a robust interface between a silica fiber and PVA hydrogel through sequential immersion in a 10 wt. % PVA solution, drying, and annealing. b, Schematic of the hydrogel fiber fabrication process, involving crosslinking within a silicone mold, demolding, acidification, stretching, drying, annealing, and reswelling to achieve desired structural and optical properties. c, Definition of Surface Area Ratio (SAR). d, Correlation between designed SAR and fabricated SAR for hydrogel fibers, demonstrating high fabrication precision with an R² of 0.9995. Each point corresponds to an independently fabricated fiber sample.

FIGS. 12A-12D show the synthesis of PVA hydrogel. a, Tetraethyl orthosilicate (TEOS) was added to a 10 wt. % PVA solution and homogenized, forming an oil-water phase in PVA-TEOS. HCl was gradually added, catalyzing the hydrolysis of TEOS to Si—OR groups at 60° C. for 12 hours. The PVA-TEOS solution was then degassed and mixed in a planetary mixer. Separately, GA was added to a 10 wt. % PVA solution, follow by degassing and mixing. The two solutions were combined (1:1 weight ratio), degassed, and mixed to create precursor solutions. b, Chemical cross-linking network of PVA with TEOS and GA. c, Enhance intermolecular interaction of PVA chain though acidification. d, Introduction of nanocrystalline growth in PVA hydrogel.

FIGS. 13A-13D show the step-index hydrogel fiber optic device fabrication with claddings. a, To fabricate a compact optical device with reduced graphene oxide (rGO) claddings, the same approaches were utilized as those used for assembling silica segments and ferrules to fabricate compact optical fibers. b, Firstly, the annealed ferrule-connected fiber was re-inserted into a silicone mold with a diameter of 500 μm. Next, GA was added into a 5 wt. % PVA solution and degassed the mixture. Similarly, hydrochloric acid (HCl) was added into another 5 wt. % PVA solution and degassed. The two solutions were then mixed with a graphene oxide solution (weight ratio of 1:1:1) and degassed. The resulting mixture of PVA-GA-HCl-rGO was infused into the 500 μm silicone mold and allowed to cross-link at room temperature for 4 hours, thereby forming a cladding layer around the optical fiber. To elute the core-cladding optical device, dichloromethane (DCM) was used. Subsequently, the compact optical device with the core-cladding was incubated in a 20 mL aqueous solution containing 1 g of sodium hydroxide and 0.3 g of sodium hydrosulfite at 40° C. for 1 minute to achieve a graphene cladding. Throughout the process, consistent mixing parameters were maintained using a planetary mixer at 2000 rpm for 1 minute under a vacuum of 16 kPa. c, A schematic illustration of light transmission in a step-index hydrogel fiber. d, Schematic illustrations and representative photographs of a PVA-TEOS-GA core hydrogel fiber, a PVA-TEOS-GA core-plain-cladding hydrogel fiber, and a PVA-TEOS-GA core-reduced reduced graphene oxide (rGO, 0.21 wt. %)-cladding fiber. Scale: 200 μm.

FIGS. 14A-14D show the mechanical properties of PVA hydrogel fibers. a, PVA hydrogel fibers' elastic modulus and stretchability calculated from tensile tests in water. One-way ANOVA and Tukey's multiple comparisons test were used to determine the statistical significance of elastic modulus: (F_{4,34}=30.07, ****p<0.0001) and stretchability: (F_{4,34}=1.040, n.s., p=0.4009), respectively. Mean±s.d., n=7-8 independent hydrogel samples. b, Representative strass-strain curves of PVA hydrogel fibers with different TEOS concentrations (0%-4%) tested in water. b, Elastic modulus and stretchability (%) calculated from tensile tests in air. (One-way ANOVA and Tukey's multiple comparisons tests were used for elastic modulus (F_{4,15}=20.51, ***p<0.0001) and stretchability (F_{4,15}=1.492, n.s. p=0.2543). Mean±s.d., n=4 independent hydrogel fibers. d, Representative strass-strain curves of PVA hydrogel fibers with different TEOS concentrations (0%-4%) tested in air with moisture.

FIGS. 15A-15F show the bending stiffness of PVA hydrogel fibers. a, An illustration of a hydrogel fiber inserted into a brain region, anchored at one end to the skull. This design allows the fiber to flexibly adapt to brain movements, facilitated by its low bending stiffness. b, Left: Insertion of a PVA optical fiber probe under dehydrated status into the phantom brain (0.6% agarose). Scale: 2.5 mm. Right: Buckling of an optical fiber probe under hydrated status during the insertion into the phantom brain (0.6% agarose). Scale: 2.5 mm. c, A schematic of the cantilever beam bending model, which provides a framework for assessing the bending stiffness of the hydrogel fiber. c, Bending stiffness of PVA hydrogel fibers (3 wt. % TEOS, mean±s.d., n=4 independent hydrogel fibers) with identical cross-sections in comparison with silica fiber (diameter: 200 μm, mean±s.d., n=3 independent samples). d, Comparison of bending stiffness commercial silica fibers and PVA hydrogel fibers. mean±s.d., each dot represents one individual sample. e, Axial bending stiffness of various soft material-based neural probes. Probes with k<100 N/m are considered compliant, while probes with k>1000 N/m are considered stiff^{85, 176, 179-183}.

FIGS. 16A-16D show the stability and cytotoxicity of PVA hydrogel fibers. a, Diameters of PVA hydrogel fibers (4% TEOS, 800 μm mold) in saline solutions (pH=6) at 37° C. over 12 weeks (One-way ANOVA and Tukey's multiple comparisons test, F₅, 18=2.124, n.s. p=0.1093). b, Diameters of PVA hydrogel fibers (4% TEOS, 800 μm mold) in saline solutions (pH=7) at 37° C. over 12 weeks (One-way ANOVA and Tukey's multiple comparisons test, F₅, 18=0.3029, n.s. p=0.9048). c, Diameters of PVA hydrogel fibers (4% TEOS, 800 μm mold) in saline solutions (pH=8) at 37° C. over 12 weeks (One-way ANOVA and Tukey's multiple comparisons test, F₅, 18=0.3089, n.s. p=0.9011). All the plots are presented as mean±s.d., n=4 independent hydrogel fibers. d, Cytotoxicity assessment of PVA hydrogel fibers. Hydrogel fibers were incubated with Human Embryonic Kidney (HEK) 293 cell cultures. Calcein-AM (green) was used to stain living cells and ethidium homodimer-1 (red) was used to stain dead cells. Cell death rates are presented as mean±standard error (s.e.m.), Two-tailed unpaired student's t-test was used: F=3.570, t=1.531, df=15, α=0.05, n.s. p=0.1466, n=8-9 microscopy measurements from 3 independent culture samples. Scale: 50 μm.

FIGS. 17A-17H show the stability of CNTs-PVA hydrogel fibers. Accelerated aging tests of TRAIN hydrogel microelectrodes (0.16 wt % CNTs, trained with 10% strain for 5000 cycles) at in artificial cerebrospinal fluid at 45° C. for 5 weeks: a, incubation in acidic environment (pH=4). b, incubation in neutral environment (pH=7). c, incubation in acidic environment (pH=10). One-way ANOVA was used to determine statistical differences: pH=4, F_{1.418, 4.253}=0.0754, n.s. p=0.8703. pH=7, F_{1.321, 3.964}=0.6095, n.s. p=0.5242. pH=10, F_{1.324, 3.971}=0.3738, n.s. p=0.6313. Each circle indicates one individual TRAIN hydrogel microelectrode (n=4). a, Impedance at 1 kHz of untrained CNTs-PVA hydrogel microelectrodes (3% TEOS with 0.16% CNT) before and after 14 days incubation in brains of mice (n=2). Paired t-test, n.s. p=0.1544. b, Impedance at 1 kHz of untrained CNTs-PVA hydrogel microelectrodes (3% TEOS with 0.16% CNT) before and after 14 days incubation (37° C.) artificial cerebrospinal fluid (aCSF). Paired t-test, n.s. p=0.6321. c, Impedance at 1 kHz of untrained CNTs-PVA hydrogel microelectrodes (3% TEOS with 0.16% CNT) before and after 14 days incubation (37° C.) aCSF with 1% bovine serum albumin (BSA). Paired t-test, n.s. p=0.3796. Each dot represent one individual microelectrode, mean±s.d. g-h, Elastic modulus (g) and stretchability (h) of aCSF incubated TRAIN hydrogel microelectrodes after 4 weeks under different pH values (4, 7, 10).

FIGS. 18A and 18B show the formation of micro canals in PVA hydrogel fibers. a, A illustration of creating micro canals through directional freezing and freeze drying in PVA hydrogel optical fibers. b, Representative SEM images at the cross-sections of micro patterned PVA hydrogel fibers with different lengths of canals associated with fiber diameter. Scale: 100 μm.

FIGS. 19A-19C show the incorporation of a microfluidic channel in PVA hydrogel fiber. a, A schematic of integrating a 50 μm microfluidic channel in the cladding layer of a PVA hydrogel fiber via molding and extrusion. b, Integrated optical probe device features a microfluidic channel with ink delivery for visualization. c, Representative SEM images at the cross-section and side face of the integrated device with one optical wave guide and microfluidic channel.

FIGS. 20A-20H shows the mechanical properties of SiO₂ coated PVA hydrogel fibers. a, Representative photographs of coated and uncoated PVA hydrogel fibers wrapped around a stick, demonstrating their flexibility and stretchability. Scale bar: 3 mm; b, Representative stress-strain curves of PVA hydrogel fibers (Uncoated fiber, coated fiber, and coated fibers with acid treatment and 45° C. incubation); c, schematic of coated PVA hydrogel fiber; d-e, elastic modulus (d) and stretchability (e) of PVA hydrogel fibers (coated fiber, and coated fibers under 37° C. pH=4 and 45° C. pH=7 for 1 week). One-way ANOVA and Tukey's multiple comparisons test were used to determine the statistical significance of elastic modulus: F(2, 15)=4.428, *p=0.0308 and stretchability: F(2, 15)=1.236, n.s.; f, schematic of uncoated PVA hydrogel fiber; g-h, elastic modulus (g) and stretchability (h) of PVA hydrogel fibers (Uncoated fiber, and uncoated fibers with acid treatment and 45° C.-incubation). One-way ANOVA and Tukey's multiple comparisons test: elastic modulus: F(2, 15)=21.81, ****p<0.0001; stretchability: F(2, 15)=15.89, ***p=0.0002. All the data sets are presented as mean±SD, each dot represents one independent fiber sample.

FIGS. 21A-21E show the COMPACT strategy for hydrogel miniaturization. a Schematic illustration of hydrogel network of metamorphic polymers' amorphous-crystal transition (COMPACT). COMPACT treatment includes cross-linking with both glutaraldehyde (GA) and tetraethyl orthosilicate (TEOS), acidification, and mechanical stretching. b-d, Representative photographs and water contents of TEOS-GA cross-linked polyvinyl alcohol (PVA) hydrogel with COMPACT treatment (+) and GA cross-linked hydrogel without acidification and stretching (−) at the pristine state (b), desiccated state (c) and rehydrated state (d). Grid size: 5 mm.

FIG. 22 shows the COMPACT opto-electrode (optrode) fabrication. The optrode contains one optical waveguide and two COMPACT hydrogel microelectrodes.

FIG. 23 shows the COMPACT hydrogel microelectrode fabrication.

FIG. 24 shows fabrications of optical probes with a functional conductive layer for opto-electronics application.

FIGS. 25A-25F show the COMPACT strategy for hydrogel miniaturization. f Shrinking behaviors of TEOS-GA cross-linked PVA (4 wt. % TEOS) hydrogel film with acidification treatment. Film thickness is quantified as mean±s.d. (Two-tailed paired student's t-test, F=56.78, t=8.455, df=10, α=0.05, ***p=0.0004, n=6 independent hydrogel films). g Shrinking behaviors of COMPACT hydrogel fibers (1-4 wt. % TEOS and 200% stretching). Hydrogel fibers' length and diameter are quantified as mean±s.d. (n=4 independent hydrogel fibers). h Shrinking behaviors of cross-linked hydrogel cylinders. The volume of TEOS-GA cross-linked hydrogel cylinders (4 wt. % TEOS) and with acidification treatment and GA cross-linked hydrogel cylinders without acidification treatment are compared with mean±s.d. (Two-tailed unpaired student's t-test, F=6.084, t=3.316, df=6, α=0.05, *p=0.0161, n=4 independent hydrogel cylinders). i Fourier transform infrared (FTIR) spectroscopy of COMPACT (−) and COMPACT (+) hydrogels. j Differential scanning calorimetry (DSC) profiles of COMPACT (−) and COMPACT (+) and their crystallinity percentages. k Small-angle X-ray (SAXS) and wide-angle X-ray (WAXS) results of hydrogel materials in the desiccated state (mean±s.d., n=3-6 independent hydrogel samples). Upper inset: schematic illustration of nanocrystalline distance change before and after stretching, middle inset: SAXS 2D patterns, lower inset: WAXS 2D patterns.

FIGS. 26A and 26B show wide-angle X-ray Scattering (WAXS) 2D spectra of nanocrystalline domain orientations in hydrogel fibers. a, Representative 2-dimensional WAXS spectra (vertical measurement) collected from 0% stretch, 100% stretch, 200% stretch COMPACT (−), and COMPACT (+) hydrogels, respectively. b, Representative 2-dimensional WAXS spectra (horizontal measurement) collected from 0% stretch, 100% stretch, 200% stretch COMPACT (−), and COMPACT (+) hydrogels, respectively. Each hydrogel fiber was repeated three times with similar results.

FIGS. 27A and 27B show controllable hydrogel fiber fabrication and its properties. a, A shrinking diagram of COMPACT (+) hydrogel fibers. The samples shaded in red areas are treated with acidification. Mean±s.d. n=3-4 independent hydrogel fibers. b, Shrinking behaviors of COMPACT hydrogel fibers (4 wt. % TEOS) prepared in different sizes of molds. One-way ANOVA and Tukey's multiple comparisons test (F_{3,12}=0.9543, n.s. not significant, p=0.4455). Mean±s.d. n=4 independent hydrogel fibers.

FIGS. 28A-280 show integrated multifunctional hydrogel neural probes. a, A representative photograph of a carbon nanotubes (CNTs)-PVA hydrogel electrode as compared with a piece of human hair. Scale: 300 μm. b A transmission electron microscopy (TEM) image of CNTs (repeated 10 times with similar results). Scale: 200 nm. c Impedance at 1 KHz and diameters of the electrodes fabricated with different stretching percentages (mean±s.d., n=3-4 independent hydrogel electrodes). d Impedance at 1 kHz of electrodes fabricated with different CNTs concentrations (mean±s.d., n=3-5 independent hydrogel electrodes). e Impedance at 1 kHz of electrodes and diameters of the electrode fabricated with different sizes of molds (mean±s.d., n=4 independent hydrogel electrodes). f Stability assessment on impedance and diameters of hydrogel electrodes incubated in PBS at 37° C. (mean±s.d., n=4 independent hydrogel electrodes). g A schematic illustration of electrical recordings from mouse VTA with a CNTs-PVA electrode bundle (5 electrodes). h Representative electrophysiology recording signals from mouse VTA with CNTs-PVA hydrogel electrodes. i A representative sorted neural spiking signal. j A representative scanning electron microscopy (SEM) image at the cross-section of an integrated multifunctional neural probe containing a hydrogel optical core and two CNTs-PVA hydrogel electrodes (repeated 10 times with similar results). Scale: 100 μm. k, 1, Photographs of a hydrogel optoelectronic device (optrode) before implantation and after implantation in a Thy1::ChR2-EYFP mouse brain. Scale: 2 mm. m Confocal images of the expression of ChR2-EYFP in the VTA region of the mouse. Scale: 50 μm. n Representative in vivo electrical signals recorded with optrodes upon optical stimulation (blue bars, λ=473 nm, 0.5 Hz, pulse width 50 ms, 10 mW/mm²). o Amplitudes of electrical signals recorded with optical stimulation over 10 weeks post-implantation (λ=473 nm, 0.5 Hz, pulse width 50 ms, 10 mW/mm2, mean±s.e.m., n=20-30 individual optically evoked peak measurements from 3 mice).

FIGS. 29A-29H show fatigue properties and biocompatibility of hydrogel optical probes with conductive layers. a-c, 180° peel tests to evaluate interfacial toughness of PVA materials, comparing PVA/PVA and PVA/CNTs-PVA layers. d, Mechanical stretching profile of a hydrogel optical probe with a conductive layer over 6,000 cycles at 16% strain amplitude. e, Enlarged view of the stretching profile from (d), showing individual cycle details. f, Stress-strain curves of hydrogel optical probes before and after mechanical stretching, illustrating the effects of cyclic loading on material properties. g, Structural thickness stability and electrochemical impedance of the conductive layer in PBS at 37° C. over a four-week incubation period. h, Cell cytotoxicity analysis comparing hydrogel optical probes with conductive layers to commercial silica and steel fibers, with representative fluorescence images indicating cell viability. Scale bars represent 50 μm.

### DETAILED DESCRIPTION OF THE PREFERRED EMBODIMENTS

### Example 1

### Conductive Hydrogels

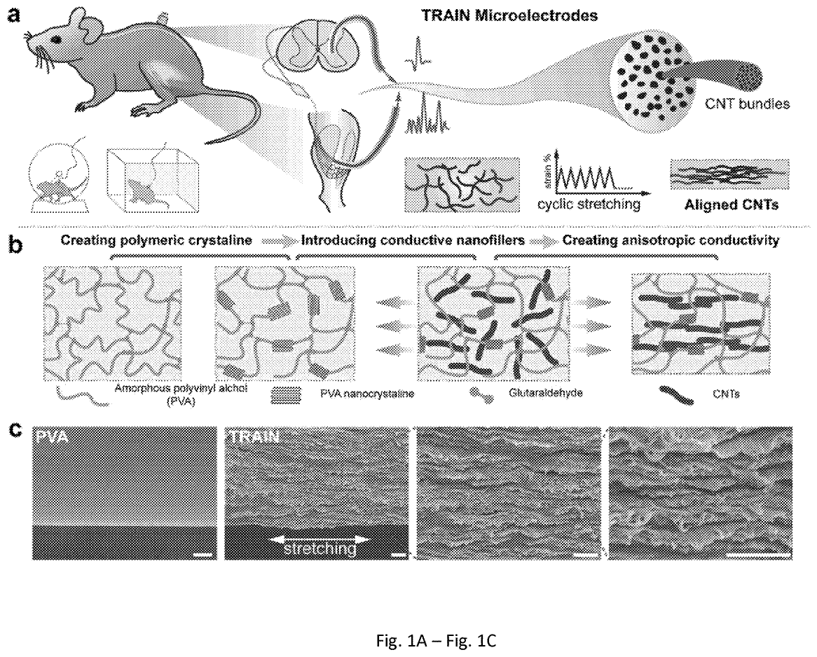

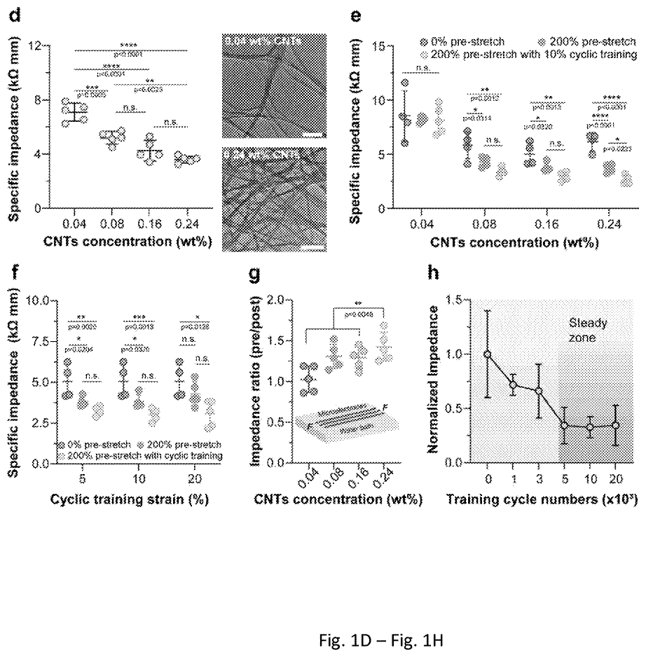

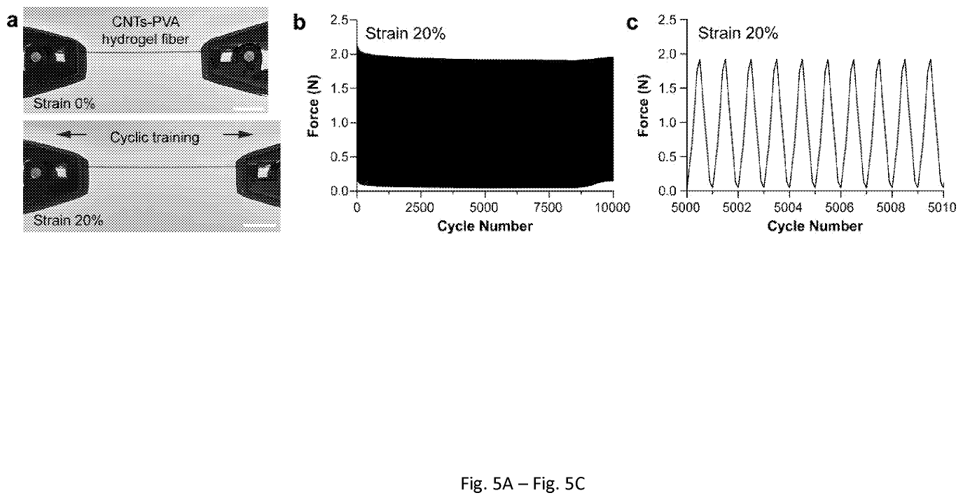

Conductive hydrogels have been applied to biosensors^{165, 200}, wearable electronics^{201, 202}, and neural interface technologies¹⁶⁸ due to mechanical compliance to complex in vivo conditions. Aiming to examine neuromuscular pathways in the spinal cord and peripheral nerves, semi-crystalline hydrogels were designed that embedded conductive nanofillers to adapt to the dynamic environment in vivo during movements (FIG. 1A). Built upon the phenomenon of stretch-induced nanocrystalline domain alignment in hydrogels^{150, 158}, the cross-linked PVA hydrogels were utilized as the matrix and CNTs introduced with the aspect ratio of 2,000-10,000:1 as the conductive nanofillers to create conductive hydrogel microelectrodes for in vivo electrical recordings. The operative hypothesis centered on the polymeric nanocrystalline of the hydrogels supporting a durable elastic substrate while the reconstruction of conductive percolative networks through nano-fillers re-alignment provides robust conductivity under stretching (FIGS. 1A-1B). CNTs were incorporated during hydrogel chemical cross-linking and then subjected CNTs-PVA hydrogels to cyclic stretching (20% strain for 10,000 cycles, FIG. 5). Scanning electron microscopy results indicated a reconfiguration of CNTs bundle structures, which aligned along the direction of applied strain from (FIG. 1C).

To examine whether these microstructural changes correlated to their functional performance, the electrochemical impedances of the conductive nanofillers-embedded hydrogel fibers wre first characterized. The CNTs-PVA hydrogel fibers were prepared with a series of CNTs concentrations in the range of 0.04 wt % to 0.24 wt %, incorporated into a 10 wt % PVA hydrogel matrix and examined their electrochemical impedance spectroscopy results (EIS, FIG. 1D). At a 0.04 wt % CNT inclusion, the impedance measured approximately 7.09±0.69 kΩ mm at 1 kHz, which is a commonly used frequency for single-neuron activity recording. These results suggest that this nanofiller concentration surpasses the percolation threshold^{136, 124, 250}. To compare the electrical performance of the CNTs-PVA hydrogel fibers across varying CNT loadings, the impedance values were normalized relative to the cross-sectional area and the effective length of the uninsulated fibers under examination. This analysis revealed a decrease in the specific impedance with an increase in CNTs content, with values declining from 7.09±0.69 kΩ mm to 3.64±0.26 kΩ mm, thereby indicating the role of CNTs in contributing to the conductivity of the hydrogel fibers (FIG. 1D).

The effect of stretching on CNTs-PVA hydrogels impedance change was then examined. Two types of stretching condition were included in the evaluation: Employing stretching during the hydrogel drying and annealing processes, which was denoted as “pre-stretch” and cyclic stretching under water when the hydrogel fibers underwent drying, annealing and re-hydration. To compare the stretching effect, a group of CNTs-PVA hydrogel fibers (0.04 wt %, 0.08 wt %, 0.16 wt % and 0.24 wt % CNTs in 10 wt % PVA) were employed with a 200% pre-stretch, a group with the combination of 200% pre-stretch with subsequent cyclic training (denoted as TRAIN hydrogel, 10% strain for 5,000 cycles under water), and a group of unstretched samples as the control (FIG. 1E). As a representative example, for fibers with a CNTs loading of 0.16 wt % and a diameter of 187±13 μm, cyclic stretching not only reduced impedance from 5.06±1.02 kΩ mm (unstretched fibers) to 3.00±0.37 kΩ·mm but also yielded a lower impedance than fibers that underwent only a single 200% pre-stretch with the impedance of 3.81±0.41 kΩ mm (FIG. 1E). To further explore the impact of mechanical training, Hooke's law²⁰³ was adhered to and the first 20% of strain defined as the elastic zone for establishing training strains in the CNTs-PVA hydrogels and observed a general enhancement in conductivity across all applied strains (FIG. 1F). To systematically assess the influence of mechanical stretching with different CNTs loadings, the impedance ratio of the hydrogel fibers was measured before and after 5,000 cycles of stretching and found over 1.03 ratio across all the groups, indicating a consistent effect of the mechanical stretching on the electrochemical impedance improvement (FIG. 1G).

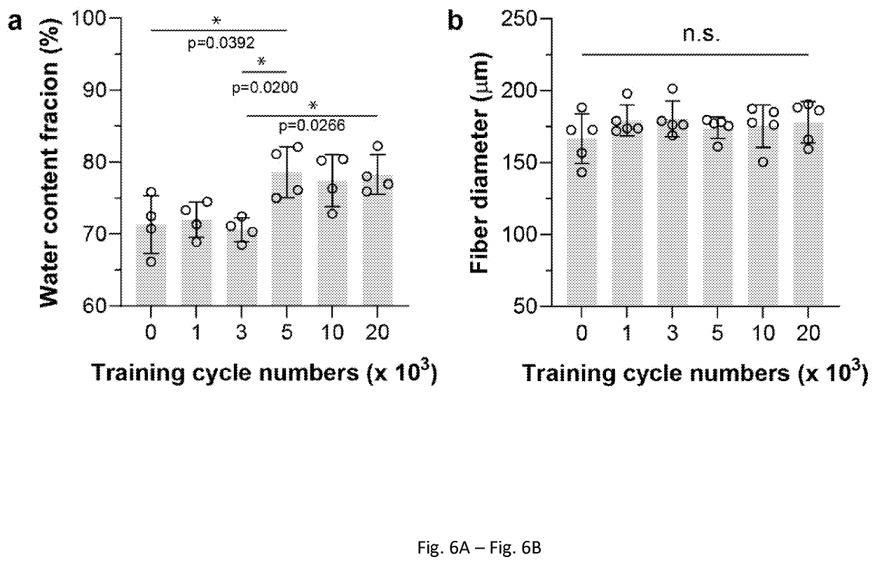

In alignment with the reported phenomenon of shakedown after prolonged cycling in tough hydrogels²⁰⁴, extending the stretching processes of the CNTs-PVA hydrogel fibers from 20,000 cycles with 20% strains resulted in a steady status after 5,000 cycles (FIG. 1H). The electrochemical impedance of each CNTs-PVA fiber was measured and these measurements compared to the original fibers without any stretching. The impedance continued to decrease during the cycling process until it stabilized at approximately 5,000 cycles, maintaining 35.64±18.62% of the original impedance. The water content faction of TRAIN CNTs-PVA hydrogel fibers reached a steady level at approximately 5,000 cycles (78.6±3.5%, FIG. 6). Given that hydrogel microelectrodes will undergo repetitive stretching conditions in vivo, maintaining the impedance and water content fraction of CNTs-PVA hydrogel fibers within this steady region is crucial for ensuring the stable performance of hydrogel electrodes in detecting electrophysiological signals.

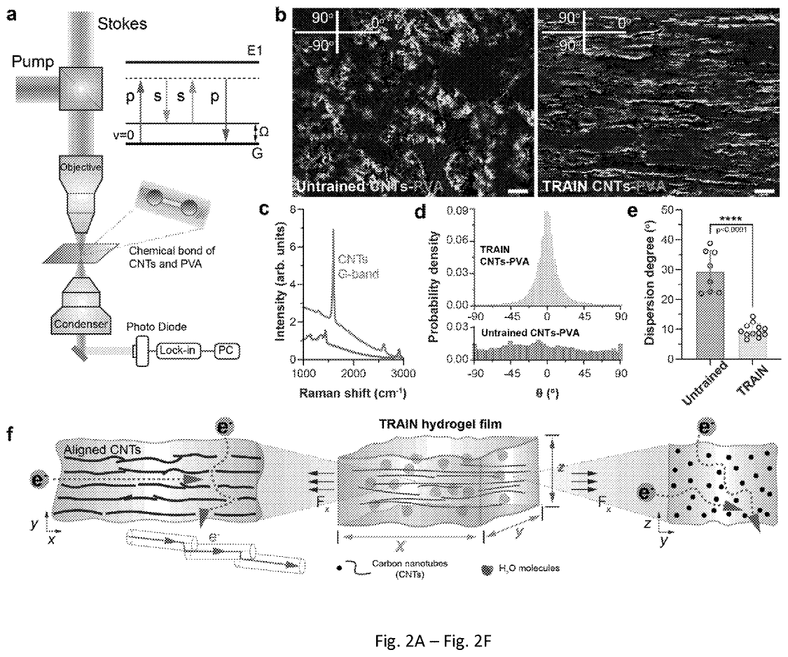

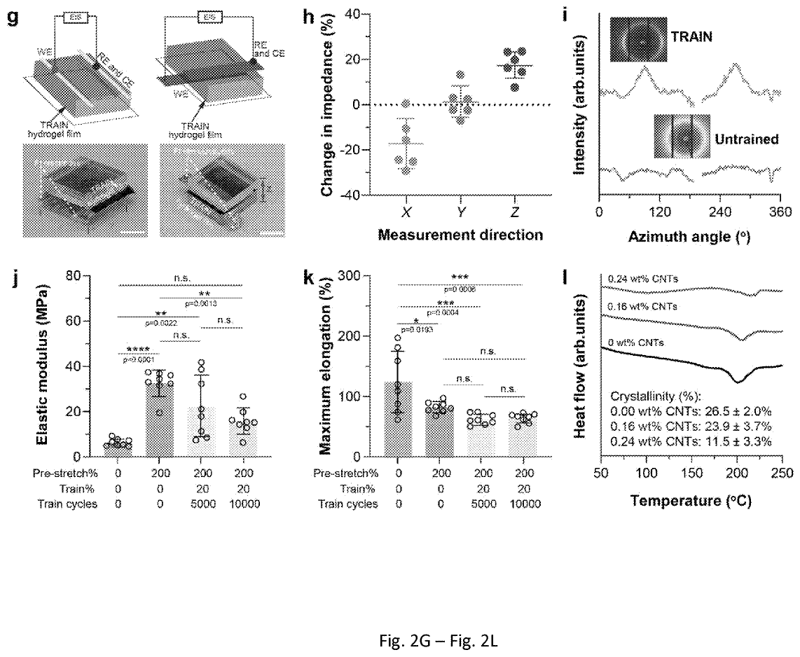

Tension Reinforcement for AnIsotropic Nano-orientation (TRAIN) strategy. To investigate the underlying mechanisms in the observed conductivity improvement after stretching, the morphology change of conductive fillers in hydrogel matrix was measured after stretching treatment. Customized stimulated Raman scattering (SRS) microscopy was applied to the CNTs-PVA hydrogels to visualize the alignment of CNTs within the PVA hydrogel matrix with chemical bond information (FIG. 2A-2B). SRS employs coherent amplification via a Pump beam and a Stokes beam, resulting in a substantial enhancement of the Raman signal strength compared to the relatively weak spontaneous Raman scattering process^{205, 206}. The featured G-band of CNTs was first detected at 1596 cm⁻¹ as the indicative C—C bond^{207, 208} (FIG. 2C). From the SRS microscopy images, following 10,000 cycles of cyclic stretching at a 20% strain, CNTs within the PVA hydrogels were preferentially aligned along the axis of stretching (FIG. 2B, 0°, denoted as TRAIN hydrogels). This realignment contrasted with the random orientation seen in the untrained CNTs-PVA hydrogels (FIGS. 2B and 2D). To quantitatively assess the realignment of CNTs, an evaluative method was adopted to measure dispersion degree metric²⁰⁹, and the results indicated a significant reduction in the dispersion degrees in TRAIN hydrogels (9.60±2.22°) as compared to their untrained counterparts (29.32±6.98°) (FIG. 2E). These results supported the hypothesis that the cyclic stretching led to the re-alignment of nano-fillers in elastic hydrogel matrix.

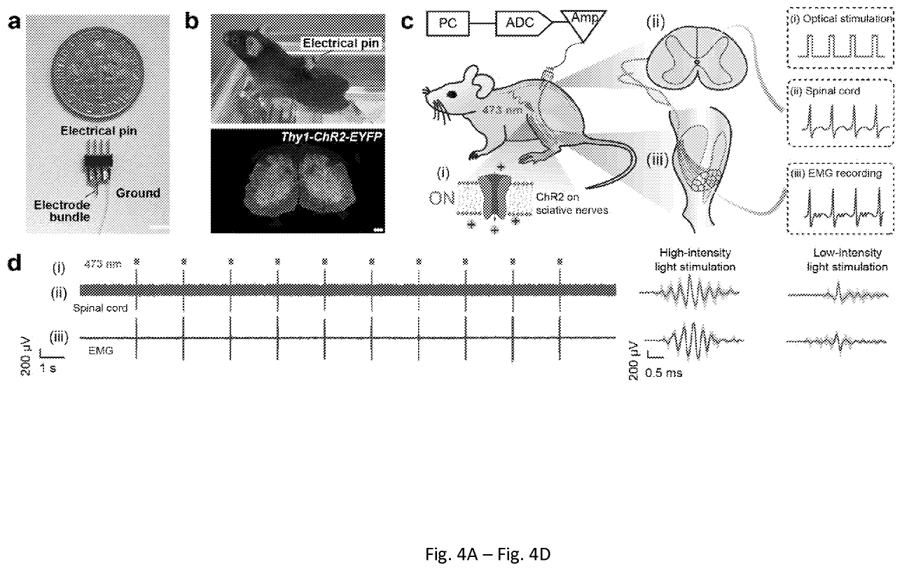

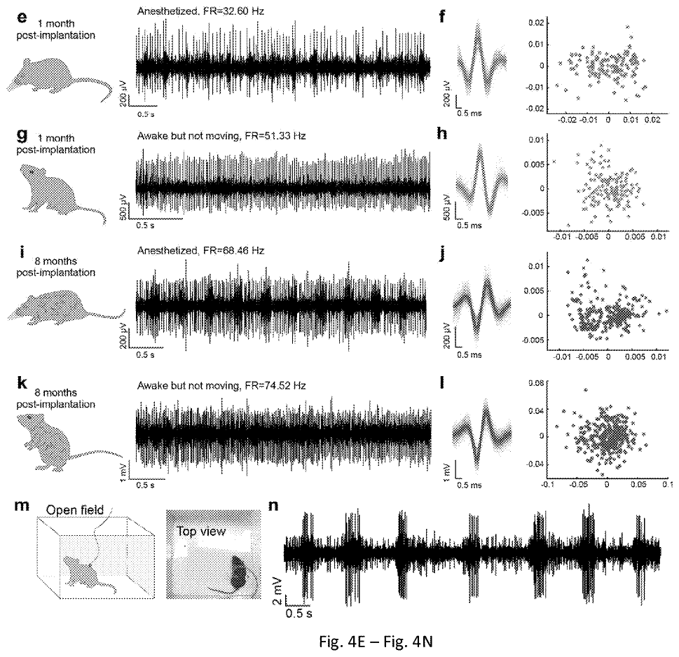

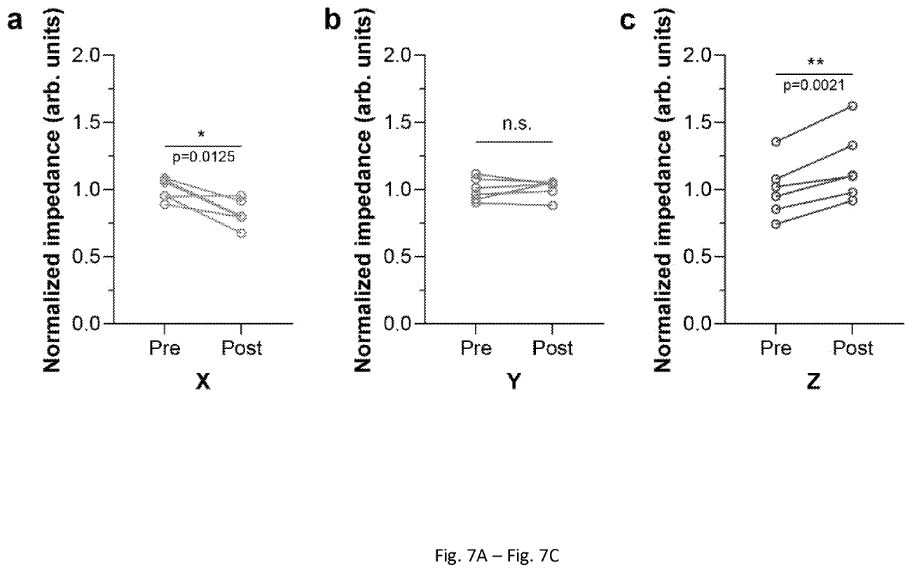

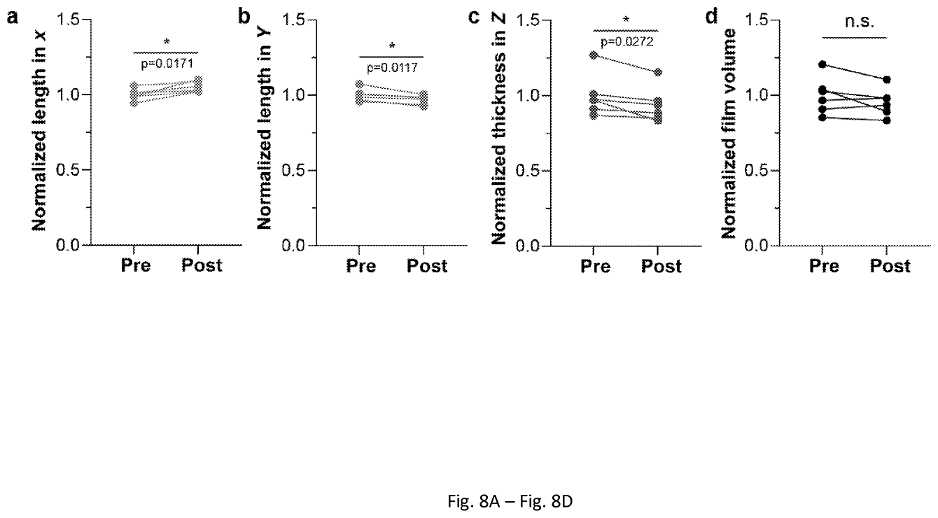

Based on morphological observations of the nanofillers, whether such structural re-alignment leads to axis-dependent impedance changes was explored. CNTs-PVA films (2 cm×2 cm×180 μm, CNTs concentrations of 0.16 wt %) were prepared and cyclic stretching applied (10% strain for 10,000 cycles) to assess impedance change along different axes. Impedance along three orthogonal vectors was defined: the x-axis, aligned parallel to the stretch, and the y and z-axes, perpendicular to the stretching direction (FIG. 2F). To measure the impedance along these respective directions, an electrochemical cell connected to a potentiostat was designed. For the x and y axes, impedance was measured using two platinum electrodes positioned on the film's surface (FIG. 2G, left). Conversely, for the z-axis, impedance measurement involved enveloping the film between two platinum films, covering the full area on opposing surfaces (FIG. 2G, right). The impedance of hydrogel films was then normalized based on the distance between electrodes and the contact area of the platinum films. Consistent with the observations in CNTs-PVA hydrogel fibers (FIGS. 4E and 4F), a 17.43±11.12% impedance decrease was observed, from 12.02±0.96 kΩ mm to 9.88±1.19 kΩ mm along the stretching direction (x-axis), with no significant change on the y-axis and a 17.49±6.04% increase along the z-axis (FIG. 2H and FIG. 7). Along with the change in impedance, the dimensions in x, y and z directions changed post-training, yet the volume of TRAIN CNTs-PVA hydrogel films remained constant (FIG. 8). Consequently, evidence of the anisotropic impedance enhancement along the stretching direction and the re-alignment of the conductive nano-fillers CNTs within the PVA matrix was observed, resulting from the re-constructed percolative network.

Similar as previously reported phenomena in semi-crystalline hydrogels^{157, 158}. The re-orientation of polymeric nanocrystalline domains in TRAIN hydrogel fibers was also observed using Wide Angle X-ray Scattering (WAXS) (FIG. 2I). These results indicated a global re-alignment of nanostructures in CNTs-PVA hydrogels under stretching.

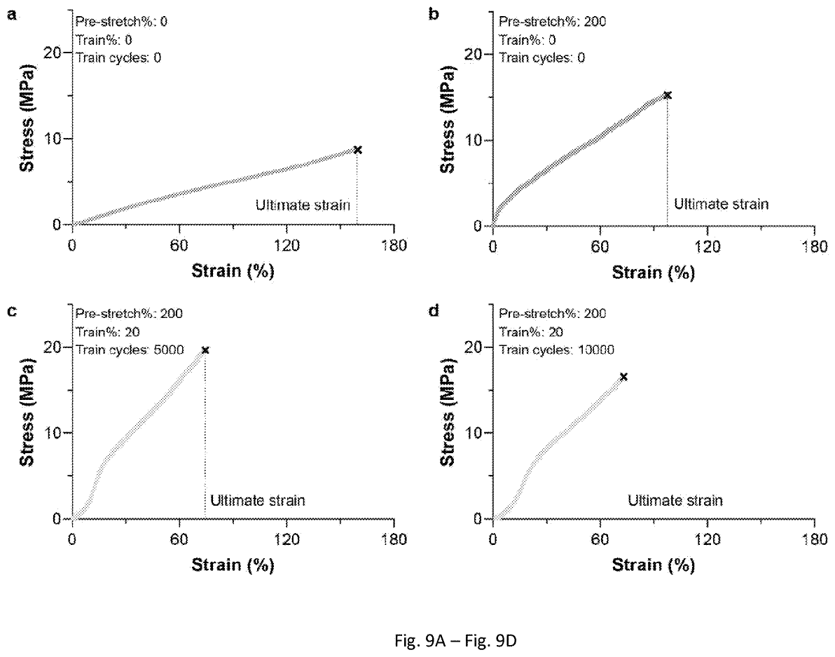

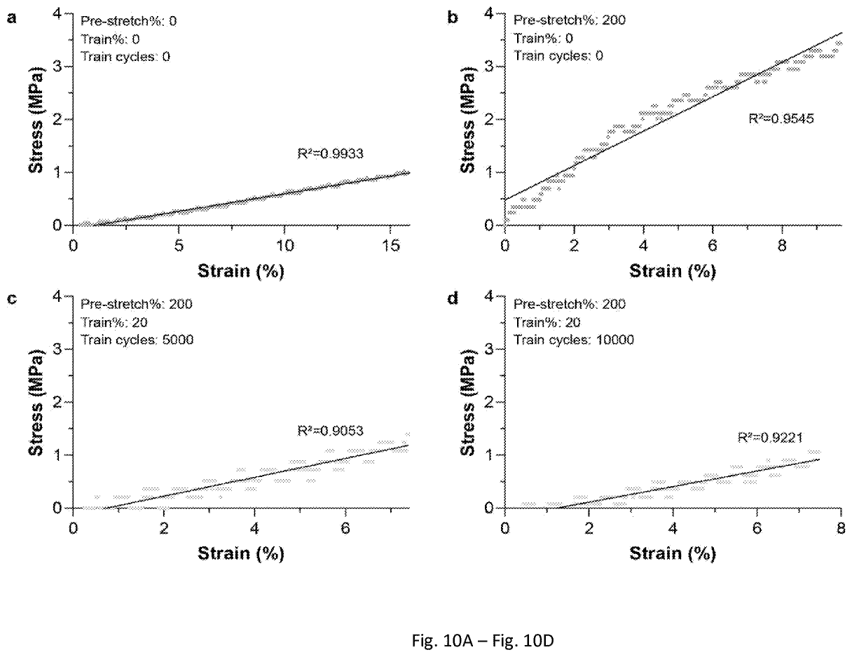

Mechanical properties of TRAIN CNTs-PVA hydrogel fibers. While the conductive nanofiller CNTs contributed to the electrical characteristics of CNTs-PVA hydrogel fibers (FIGS. 1D-1G), the stretching treatment affects the mechanical properties was investigated, especially under consideration for in vivo tissue movements. The single tensile test results revealed that 0.16 wt % CNTs-PVA hydrogel fibers without training exhibited a relatively low elastic modulus of 6.45±1.63 MPa (FIGS. 2J, 9 and 10), along with a maximum elongation of 124.09±50.46% (FIGS. 2K and 9). However, the elastic modulus of the fibers significantly increased to 32.74±5.74 MPa post a 200% pre-stretch treatment during drying and annealing, due to the internal-stress induced rigid nanofillers' alignment, similar to previous research^{210, 211}. However, further cyclic stretching underwater led to a reduction in the elastic modulus to 15.97±5.82 MPa (FIG. 2J), potentially attributed from the shakedown phenomenon in hydrogels²⁰⁴. Fibers subjected to the TRAIN strategy (20% strain for 10,000 cycles) showed an elastic modulus comparable to that of the untrained fibers, indicating that the TRAIN method did not compromise the inherent softness of the PVA hydrogel, an advantageous feature for motion-adaptive in vivo applications. To delve deeper into the effects of CNTs on the PVA hydrogel nanostructures, Differential Scanning calorimetry (DSC) was employed to characterize the crystallinity within the TRAIN hydrogel fibers. A shift in the characteristic endothermic peak of PVA nanocrystals was observed, and upon quantifying the enthalpy changes for different CNTs concentrations (0.16 wt % and 0.24 wt %), it was determined that CNTs could modulate nanocrystalline growth within the PVA matrix¹⁵⁷, reducing crystallinity from 26.5% to 11.5% (FIG. 2I). This observation aligns with the report of introducing nano-fillers inhibits the nano-crystallization in hydrogels²¹².

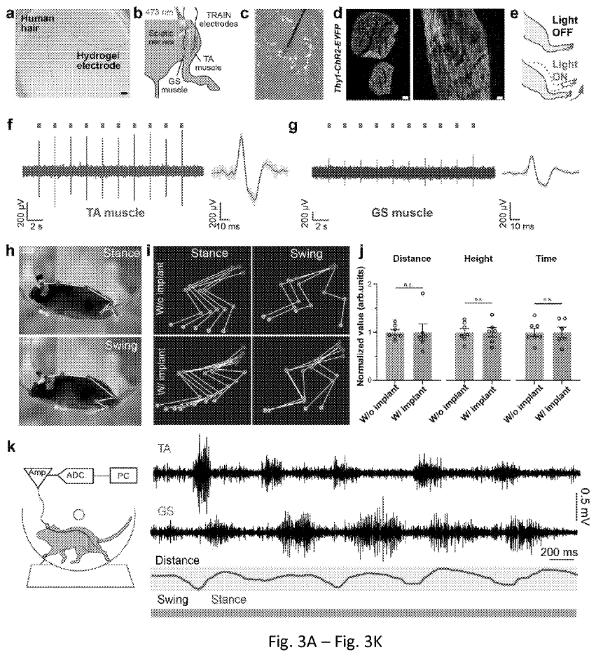

To facilitate in vivo animal studies, thin CNTs-PVA hydrogel fibers (187±13 μm in diameter, FIG. 3A) were fabricated into electrical devices with a layer of styrene-ethylene-butylene-styrene (SEBS) thermoplastic elastomer as an insulating coat via the dip-coating method (thickness: 1.8±0.4 μm). Next, hydrogel microelectrodes were surgically implanted into the TA and GS muscles of the hindlimbs in Thy1-ChR2-EYFP mice (FIGS. 3B and 3C). These transgenic mice were selected because they intrinsically express the photo-excitable Channelrhodopsin 2 (ChR2) within their nervous systems, which facilitates neuronal manipulation and recording from live animals. The expression of ChR2 was confirmed in mouse sciatic nerves through confocal microscopy (FIG. 3D). With anesthetized Thy1-ChR2-EYFP mice, blue light stimulation (473 nm) was applied transdermally to the hindlimb muscles, inducing neural excitation and subsequent muscle contraction (FIG. 3E). Before implantation, the TRAIN hydrogel electrodes were dehydrated to enhance stiffness, facilitating easier insertion. Once implanted, the electrodes rehydrate from tissue fluids, softening to integrate more seamlessly with muscle tissues. The TRAIN hydrogel electrodes consistently captured EMG signals in both the TA and GS muscles correlated well with pulsed transdermal blue light stimulation (473 nm, 0.5 Hz, pulse width 50 ms). These EMG recordings demonstrated uniform amplitudes and waveform and exhibited high signal-to-noise ratios (SNR: 9.25 in TA), attesting to the electrodes' efficacy (FIGS. 3F and 3G).

After confirming TRAIN microelectrodes' functionality in vivo, their performance was tested in conditions involving naturalistic animal movement. As a proof-of-concept application, mouse EMG signals were collected with TRAIN microelectrodes during voluntary wheel running tests (VWRTs)^{150, 216}, a standard assay for assessing locomotor activity by labeling skeletal landmarks with a network of nodes to track the running behaviors through a markerless motion tracking algorithm, DeepLabCut^{150, 186}. The implantation of TRAIN microelectrodes in mouse TA and GS muscles was evaluated to determine whether they impaired mouse natural movement by performing gait analysis on implanted hindlimbs. Three days after the mouse recovery from the implantation surgeries, the gait of the implanted mice was compared to the sham controls in their gait kinematics during swing and stance phases, which showed no significant differences (FIGS. 3H-3J), indicating surgical implants do not adversely affect mouse basic gait parameters.

With the implanted microelectrodes remaining in the muscles over 1-week post-implantation, EMG recordings from TA and GS muscles were made simultaneously by implanting TRAIN hydrogel microelectrodes in both sites while mice moved freely during VWRT and correlated the collected EMG signals to locomotion behaviors (FIG. 3K, left). By synchronizing the EMG recordings with locomotion behaviors, a clear alternative EMG activity of TA and GS muscles (FIG. 3K, right) was observed, in line with previous studies that used conventional electrode EMG recordings²¹⁷⁻²¹⁹.

Leveraging the TRAIN hydrogel microelectrodes can detect EMG signals in freely moving animals, whether it could detect neuronal electrical activity in live spinal cords was explored. Three TRAIN microelectrodes were integrated into one miniaturized device (FIG. 4A) to detect electrical recordings simultaneously from mouse spinal cords and muscles. The miniaturized device was implanted into the mouse back region, with one electrode into the Lumbar (L) 3 spinal cord ventral horn region, where the TA muscle motor neurons are located, and the other one into hindlimb TA muscle in Thy1-ChR2-EYFP mice (FIGS. 4B and 4C). Following a three-day recovery, the Thy1-ChR2-EYFP mice were subjected to transdermal optical stimulation (473 nm, 0.5 Hz, pulse width 50 ms) under anesthesia, with simultaneous electrical recordings from both ventral horn part of the L3 spinal cord and the TA muscle (FIG. 4D). To verify that the recorded electrical signals from the spinal cord and TA muscles came from the transdermal optogenetic stimulation, the intensity of the blue light pulses was altered and the corresponding electrical signal changes compared. By isolating and overlaying each detected electric peak (n=10 individual peaks), distinct changes in the amplitudes and waveforms of the extracellular signals from the spinal cord and the EMG signals (FIG. 4D) were observed, which underscored the responsiveness of hydrogel microelectrodes.

Given these results, the capability of the electrodes to detect spontaneous neural activities from the spinal cord and stay functional over long-term observations was analyzed. The endogenous neural spikes from anesthetized mice 1-month post-implantation with a bundle of three TRAIN hydrogel microelectrodes implanted in ventral horn at L3 (FIG. 4E) was recorded. Single neuron activities were delineated using principal component analysis (PCA) (FIG. 4F). The electrodes' performance in awake mice was then examined by recording from the same group of mice using the same device. The sorted neural spikes maintained the same waveforms but with a higher firing rate (FR), from a representative FR of 32.60 Hz under anesthesia to an FR of 51.33 Hz during awake (FIGS. 4G and 4H). The TRAIN hydrogel electrodes maintained long-term stability and durability in vivo with the electrophysiological recording from mice 8 months post-implantation. The implanted hydrogel microelectrodes were still able to collect spontaneous neural activities in the mouse spinal cord with distinct single-unit waveform and amplitude (FIGS. 4I-4L). When the implanted mice were set to freely moving (FIG. 4M), a combination of burst patterns with large electrical peak clusters along with movement and tonic spikes (FIG. 4N) was observed, indicating the adaptivity and flexibility of TRAIN hydrogel electrodes for electrophysiological recordings during in vivo dynamic environments.

Beyond the traditional spinal cord surface epidural recording devices²²⁰⁻²²⁴, fiber-shaped or other penetrating microelectrodes demonstrate a capability to probe deep structures of the spinal cord with single-unit precision^{64, 225}, however, the complex motion and the fragile spinal cord tissues require further soft neural-materials interface designs to minimize tissue damage from motion and the proper motion artifact management²²⁶. Fatigue-resistant soft hydrogels offer motion-adaptive advantages by improving material-tissue mechanical matching and stretchability. In addition to the compliant interfaces, the reinforced electrical percolative networks support stable interface impedance and consistent conductive pathways to collect electrophysiological signals during dynamic conditions Moreover, such soft bioelectronics also allow minimal disruption to the natural behaviors of the experimental subjects. Using an integrated bioelectronic device to simultaneously record different sites of muscles and nervous systems in the context of behavioral tests, this technology offers direct links between nerve and muscle circuit activities.

A set of hydrogel microelectrodes was engineered with a bottom-up approach to reinforcing the percolative network of nanofillers and therefore provided adaptive tissue-material interfaces. Such hydrogel microelectrodes enable electrophysiological recording from mouse muscles and lumbar spinal cord ventral horn region under complex mechanical dynamics in vivo. The CNTs-PVA hydrogel microelectrodes exhibited anisotropic electrochemical impedance and investigated the mechanism of stretch-induced reconstruction of conductive nanofillers in a hydrogel matrix. With the retained softness and stretchability, as well as the directional conductivity, these hydrogel microelectrodes were employed in vivo, they robustly collected the electrical signals from multiple muscles simultaneously during mouse locomotion. Using an integrated device with hydrogel microelectrodes implanted in the mouse ventral horn of the spinal cord and muscle separately, the light-evoked electrical signaling in the sciatic-spinal-motor reflex arc was captured using transgenic mice. Due to the strong motion adaptation to the spinal cord tissues, the electrophysiological signals from mouse ventral horn areas were successfully recorded from various statuses, such as under anesthesia, awake, and naturally behaving. This soft nanomaterials-supported tissue-integrated bioelectronics offers a solution for collective recording from multiple sites, especially in those with severe mechanical dynamics, and a holistic understanding of neural circuits in the context of behaviors.

Fabrication of conductive hydrogel fibers. To prepare a 10 wt % polyvinyl alcohol (PVA) solution, 10 g of PVA (146,000 to 186,000 Da, 99+% hydrolyzed, Sigma Aldrich 363065) was combined with 90 g of Milli-Q water (14 MΩ·cm at 25° C.). The mixture was heated to 100° C. under continuous stirring for 5 hours, resulting in a clear and viscous solution. For the carbon nanotubes (CNTs) preparation, a stock solution of CNTs (OCSiAl, TUBALL BATT H2O 0.8% beta) was diluted with Milli-Q water in a weight ratio of 1:0.6. To prevent the aggregation of CNTs, a solution of Sodium Dodecylbenzene Sulfonate (SDBS, 95%+, Fisher Scientific D0990500G) was prepared at a concentration of 5 wt %. To fabricate chemically cross-linked CNTs-PVA hydrogel fibers, 100 μL of Glutaraldehyde (GA, 25% in water, Sigma Aldrich G6257) was added into 10 g of 10 wt. % PVA solution followed by mixing and degassing in a vacuum spinner (Musashi ARV-310). 150 μL of hydrochloric acid (HCl, 37%, Sigma-Aldrich 258148) was then added into another 10 g of 10 wt. % PVA solution with mixing and degassing followed. Next, the diluted CNTs, PVA-GA, and PVA-HCl solutions were combined on a ratio of (0.6~1.4): 1:1 with the same mixing and degassing procedure. The homogeneous solution was then infused into silicone molds (800 μm I.D., 51845K51 and 500 μm I.D., 51845K66, MacMaster-Carr) and allowed to cross-link at room temperature (RT) for 2 hours.

Dichloromethane (DCM, 99.8%, Sigma Aldrich 270997) was utilized to induce swelling in silicone molds, aiding the elution of CNTs-PVA hydrogel fibers. Post-elution, these fibers were rinsed with large amounts of Milli-Q water for 2 days to remove any residual unreacted chemicals. Following the washing step, the CNTs-PVA hydrogel fibers were immersed in HCl solution (12 mM) for 2 hours (cite COMPACT), then pre-stretched to 200% strain and dried in the air for 12 hours followed by annealing at 100° C. for 20 minutes. Finally, the fibers were reswelled in Milli-Q water for later use.

Fabrications of conductive hydrogel films. The CNTs stock solution was diluted with Milli-Q water (1:0.6 by weight) and then homogeneously dispersed with a 5 wt % SDBS solution (1:10 by weight). Subsequently, 100 μL of GA was incorporated into 10 g of a 10 wt % PVA solution. Additionally, 150 μL of HCl was added to another 10 g of 10 wt % PVA solution. Both mixtures were subjected to thorough mixing and degassing using a vacuum spinner. The diluted CNTs, PVA-GA, and PVA-HCl, were then combined in a ratio of 1.4:1:1, employing identical mixing and degassing procedures. The resultant homogeneous mixture was poured into a customized mold and allowed to cross-link at room temperature for 2 hours. The resultant CNTs-PVA hydrogel films were demolded and extensively rinsed with Milli-Q water to eliminate any unreacted chemicals. The films underwent a post-treatment with an HCl solution (12 mM) followed by pre-stretching the films to 10% strain and air drying for 12 hours. The films were annealed at 100° C. for 20 minutes. Finally, these films were reswelled in Milli-Q water.

TRAIN hydrogel materials. To reorient CNTs in the PVA hydrogel matrix and fabricate TRAIN hydrogel fibers and films, cyclic stretching tests (strain-controlled mode: 5%, 10% and 20% strains, and frequency of 0.5 Hz) for 5,000-15,000 cycles using a horizontal mechanical tester (Univert, Cell Scale) with a water bath were conducted. A 4.4 N load cell (Futek) was used. The conductive hydrogel materials were stretched with a constant strain amplitude, and the force variation was recorded over time.

Electrochemical characterization of conductive hydrogel materials. Impedance of conductive hydrogel fibers and thin films was systematically assessed using an electrochemical working station (Princeton Applied Research PARSTAT 2273). A 3-electrode electrochemical sink was used to characterize conductive TRAIN hydrogel fibers with a sinusoidal driving voltage spanning a frequency range from 10 Hz to 1 MHz and an amplitude of 10 mV. The conductive hydrogel films, after undergoing repeated training, were analyzed using a specialized device in three different orientations: along the stretching direction (x-axis), across the stretching direction on the surface of the film (y-axis), and through the thickness of the film, perpendicular to the stretching direction (z-axis). For measurements along the x and y axes, impedance was determined using two platinum wires (diameter: 1.54 mm, Length: 19.47 mm, and distance 15.44 mm). The length (l) used in calculations is the 15.44 mm distance between the wires, and the area (A) is the contact area where each wire touches the film. For the Z-axis measurements, impedance was recorded using two platinum sheets (length: 19.47 mm and width: 6.41 mm). Here, l is the thickness of the film, and A is the area where each sheet contacts the film. For hydrogel fibers, l is defined as the length of fiber immersed in a chemical solution, and A is the cross-sectional area of the immersed part of the fiber. The lengths of the hydrogel fibers and films were measured using a caliper, while their thicknesses were accurately gauged using a micrometer. The measured impedance was normalized using the specific formula:

$$R = \frac{l}{A} ρ ,$$

where R is the measured impedance, l is the length of the materials that were characterized, A is the area of the materials that were characterized, ρ is the specific impedance of the conductive hydrogel materials.

Dimension measurements of hydrogel fibers. Images of hydrogel fibers were captured using a microscope (AmScope) under a bright field while submerged in water. Each fiber was examined in three distinct areas, including both ends and the middle (three individual measurements in each section). Subsequently, the Image J software was used to measure the diameter of each fiber. In addition, the length (five individual measurements) of the fibers was measured using a caliper.

SEM characterization of TRAIN hydrogel films. To investigate the structure and morphology of hydrogel films, samples were first rapidly frozen using liquid nitrogen to preserve their internal structure. For TRAIN conductive hydrogel films, they were fractured along lines parallel to the training direction to expose the relevant structural facets. For pre PVA hydrogel films, they were fractured randomly. After fracturing, the hydrogel films were sputtered with platinum to enhance conductivity and then examined using SEM (Hitachi Su5000) at an acceleration voltage of 4 kV. For representative SEM images, each sample was repeated ten times with similar results.

TEM imaging of CNTs. TEM images were acquired using a transmission electron microscope (FEI Tecnai 12, 120 kV). CNTs were diluted in a range from 1:3.3 to 1:10 with Milli-Q water and subsequently deposited onto a copper grid (Sigma-Aldrich FCF200-Cu) for imaging purposes. To ensure the reproducibility of the observations, each sample was imaged 6 times, consistently yielding similar results.

Raman Spectroscopy. Raman spectra of TRAIN CNTs-PVA and PVA hydrogel films were acquired using a confocal Raman microscope (Renishaw InVia, 785-nm NIR laser, 20× objective lens). Prior to spectral collection, the films were mounted on glass slides enveloped in a reflective substrate. The acquisition resolution was 0.7 cm-1 and the integration time per acquisition was 10 seconds. For the TRAIN CNTs-PVA hydrogel films, to prevent thermal degradation of the CNTs, the laser power was reduced to 0.1%, resulting in an effective power of approximately 0.57 mW at the focal point of the sample. In contrast, the PVA hydrogel samples were analyzed under 10% laser power, corresponding to an effective power of about 10 mW at the sample focus. All spectra were recorded and subsequently analyzed using Renishaw's WIRE 4.1 software, ensuring precise and reproducible data for further interpretation.

SRS microscopy. To prepare samples for SRS imaging, TRAIN and untrained CNTs-PVA hydrogel films (0.16% and 0.24% CNTs) were imbedded in O.C.T compounds (−80° C. for 2 hours) and then sliced with a 20 μm thickness using a cryo-slicing microtome (Leica CM1950). The sliced samples were then mounted onto glass slides for later imaging. A customized SRS setup was used to image for specific CNT signals in the TRAIN and untrained CNTs-PVA hydrogel samples. Insight X3, (Spectra-Physics), a dual channel/wavelength, pulsed laser was used to generate the SRS signals. The modulated SRL signal (stimulated Raman loss) was captured with FDS1010 Si-photodiode (Thorlabs Inc.), which was then processed/demodulated by a HF2LI lock-in amplifier (Zurich Instruments). Scan image software was used as an overall image-capturing system to generate the final digital SRS images with 1024×1024 pixel data density. Imaging was performed under a 25× water immersion objective with a 3× zoom, capturing an effective sample area of 233×233 μm². The strokes and pump beams were set to an average power of 100 mW and 30 mW, respectively. The strokes beam is fixed at 1045 nm, and the tunable pump beam was set to 805, 809, 880 and 896 nm to obtain 4 sets of SRS images for each sample and location. SRS images were taken at 896 nm for the CNTs selective chemical imaging (based on Raman spectra) and 805 nm for the PVA hydrogel imaging. Images were also taken at 88 0 nm and 809 nm for off-peak (background) values, of CNTs and PVA hydrogel respectively. The collected images were processed, background subtracted, and overlaid with pseudo-colors for visualization, using Fiji ImageJ software. To ensure the reproducibility of the observations, each sample was imaged 6 times, consistently yielding similar results.

Mechanical tests of conductive hydrogel fibers. Tensile test machine (Univert, Cell Scale) was employed to stretch the hydrogel fibers a rate of 1 mm/s under water. The nominal stress was calculated using the formula σ=F/A, where F denotes the recorded force and A denotes the cross-sectional area of the fibers in the hydrated state. The strain was calculated using ϵ=ΔL/L, where ΔL represents the displacement, and L represents the gauge length. The elastic moduli € were determined by calculating the average slope of the stress-strain relationship in the first 10% of applied strain using linear regression. The maximum elongation (%) of the fibers was reported at the point of fracture in the stress-strain curve.

X-ray scattering. X-ray scattering experiments were conducted using the SAXSLAB GANESHA 300XL instrument, equipped with a Dectris Pilatus 300K 2D CMOS photon counting detector (measuring 83.8×106.5 mm²). Wide angle X-ray (WAXS) measurements employed a 2 mm beamstop, and each sample was exposed for a duration of 300 s.

Measurement of crystallinities. The degree of crystallinity of hydrogel fibers and materials was assessed using a DSC instrument (2920 TA instrument). The PVA hydrogels were analyzed in the desiccated state. A small quantity of sample (1-15 mg) was loaded into a crucible (TA instrument T81006) and placed in a temperature-controlled DSC cell. A blank crucible served as a reference. The sample was heated from 30° C. to 300° C. in air, with a heating rate of 20° C./min. The differential heat flow to the sample and reference was recorded by the instrument. To determine the melting fusion enthalpy of endothermic peaks, heat flow (mW) over sample weight (mg) was plotted against time(s). The areas of melting endothermic peaks were integrated using TA analyze software (TA Universal Analysis). The degree of crystallinity α was estimated using the equation: α=ΔH_{f}/ΔHₘ·100%, where ΔH_{f} (J/g) was calculated from the integration of melting endothermic peaks and ΔHₘ (150 J/g) was the enthalpy of melting 100% of PVA crystallites (cite).

Insulation of hydrogel microelectrodes. To insulate the CNTs-PVA hydrogel electrodes, a dip-coating process was performed using a 20 wt. % SEBS (Styrene-Ethylene-Butylene-Styrene) solution in toluene. The SEBS solution was prepared by thoroughly dissolving SEBS in toluene with constant stirring to ensure a homogenous mixture. Prior to dip coating, the CNTs-PVA hydrogel electrodes were cleaned, straightened, and dried to eliminate any surface contaminants that might interfere with the coating process. The electrodes were then immersed into the SEBS solution at a controlled speed to ensure smooth application and prevent the formation of surface irregularities or trapped air bubbles. The depth of immersion was carefully controlled to ensure complete coverage of the electrode surface intended for insulation. After immersion, the electrodes were withdrawn with a speed of 11.8 mm/min from the solution to ensure an even coating layer. Following the dip-coating process, the electrodes were suspended vertically in a well-ventilated area to allow the toluene solvent to evaporate. Solvent evaporation was conducted under ambient conditions to promote uniform drying for 1 hour. After complete solvent evaporation, the electrodes were inspected for uniformity of the SEBS coating. If any irregularities in thickness or coverage were detected, additional dip-coating cycles were performed to ensure optimal insulation. Once the dip-coating and curing processes were complete, the electrodes were stored in a clean, dust-free environment to prevent contamination prior to their use in subsequent experiments.

Assembling of conductive hydrogel bioelectronic device. To fabricate TRAIN hydrogel electrodes, a hydrogel conductive fiber and a stainless-steel wire were dip-coated in CNTs (0.8 wt %) solution. The dip-coated hydrogel fiber and stainless-steel wire were inserted into an elastic tubing (100 μm) to create a hydrogel-steel junction. Another thin layer of silver paint was applied at the junction to enhance conductivity. UV epoxy (Norland optical adhesive) was used to seal and reinforce the junction. To insulate the TRAIN hydrogel electrodes, Styrene-ethylene-butylene-styrene (SEBS, 20 wt. %) was diluted in Toluene (Fisher Scientific T290-1) to form a uniform solution. The TRAIN hydrogel electrodes were dipped into the solution and allowed to dry in the air for 1 hour. To assemble the bioelectronic device for EMG recordings, 4 insulated TRAIN electrodes were soldered onto four pins from a six-pin connector, respectively. One insulated stainless-steel wire (50 μm) was soldered onto the last pin as the ground wire. To assemble the bioelectronic device for spinal cord recordings, 3 insulated TRAIN electrodes were soldered onto three pins from a four-pin connector, respectively. One insulated stainless-steel wire (50 μm) was soldered onto the last pin as the ground wire. The three working electrodes were twisted into a yarn and dipped coated in SEBS solution. Optical adhesive was applied to the soldering sides on the pin to provide insulation.

Experimental animals. All experiments on mice were reviewed and approved by The Institutional Animal Care and Use Committee at Binghamton University (Protocol number: 897-23) and University of Massachusetts Amherst (Protocol number: 2520). Wild-type (C57BL/6J) and Thy1-ChR2-EYFP mice (JAX stock #007612) were purchased from the Jackson Laboratory. Mice were given ad libitum access to food and water and were housed at 24° C.=1° C., with 50% relative humidity, and on a 12-h light/12-h dark cycle. All experiments were conducted during the light cycle.

In vivo implantation in muscle for anesthetic EMG recording. Thy1-ChR2-EYFP mice were anesthetized using isoflurane (1.5% induction) and continuously maintained at 1%. Supplemental heat was provided during the surgery. The fur was removed over the hindlimbs. To collect EMG signals from Thy1-ChR2-EYFP mice under anesthesia, an insulated TRAIN hydrogel electrode was inserted into the medial tibialis anterior (TA) and gastrocnemius (GN) muscles of mice hindlimbs. A reference needle electrode was inserted in an electrical unrelated region. A ground needle electrode was subcutaneously inserted into the tail. A 473 nm laser was used for transdermal optical stimulation. EMG data triggered by optogenetic activation were filtered (10 Hz to 1 kHz) collected through a DAM50 system.

In vivo implantation in muscle for freely moving EMG recording. The working electrode (PVA-CNTs hydrogel), reference electrode (PVA-CNTs hydrogel), and ground wire (stainless steel wire) were soldered on an electrode pin connector. PVA-CNT hydrogel working electrodes were led by 30-gauge needles and inserted into the mid-belly of the TA muscles of the left hindlimb in wild type (WT) mice. The ground wire and reference electrode were inserted subcutaneously in the neck-shoulder area. Electrodes and wires were routed subcutaneously through the back and the pin connector was secured on the skull by dental cement (Jet Set-4).

In vivo implantation in muscle for freely moving EMG recording. C67BL/6J mice were anesthetized with 1.5% isoflurane continuously maintained at 1%, with supplemental heat provided throughout the surgical procedure. Fur was removed from the hindlimbs and head, and the exposed skin was sanitized using iodine followed by 75% ethanol. Incisions were then made at each site.

A 6-pin device was carefully routed subcutaneously from the hindlimb to the skull. The pin connector was securely affixed to the skull using dental cement (Parkell C&B METABOND and Jet Set-4). In wild type mice, two TRAIN hydrogel electrodes were employed—one as a working electrode and the other as a reference electrode. Both electrodes were inserted into the mid-belly of the tibialis anterior (TA) muscles on the left hindlimb using 30-gauge needles. Similarly, two additional TRAIN hydrogel electrodes were inserted into the gastrocnemius (GS) muscle on the left hindlimb of the same wild type mice. A ground wire was positioned subcutaneously in the neck-shoulder area to complete the setup. The skin over the hindlimb muscles was then sutured closed using nylon sutures (Nylon 5-0).

In vivo implantation in spinal cord. Thy1-ChR2-EYFP mice were anesthetized using 1.5% isoflurane for induction and continuously maintained at 1%, and supplemental heat was provided to maintain body temperature during the surgery. The fur on the dorsum was shaved, and the skin was subsequently disinfected with povidone iodine followed by 75% alcohol. A single midline incision was made over the vertebral segments from T11 to T13, and the paraspinal muscles were dissected away to expose the underlying vertebrae. Using lateral spinal clamps, the exposed section of the spine was stabilized on a stereotaxic frame between T11 and T13. Further dissection exposed the spinal cord between the L3 and L4 vertebrae. An electronic device was then carefully positioned on the dorsal surface of the spinal cord and lowered by 400 μm into the dorsal horn to ensure precise placement. The device was secured using dental cement over the T12 and T13 vertebrae to prevent displacement (Parkell C&B METABOND and Jet Set-4). Closure of the surgical site was achieved by suturing the skin over the dorsum using Nylon 5-0 sutures.

Synchronized EMG recordings. Thy1-ChR2-EYFP mice were anesthetized using isoflurane (1.5% induction and continuously maintained at 1%). The fur was removed over the hindlimb. One TRAIN hydrogel electrode was inserted in the TA muscle, and one TRAIN hydrogel electrode was inserted into the GS muscle. One needle reference electrode was inserted in the unrelated region, and one needle ground electrode was inserted in the tail. The working electrodes (TA and GN), reference electrode and ground electrode were connected to a DAM8 system. Transdermal optical illumination on the sciatic nerves was carried out using a 473 nm laser The laser was pulsed at a frequency of 0.5 Hz with a pulse width of 50 ms during optical stimulation. Signals were digitized at 4 kHz (DI-1100, DATAQ Instruments) and filtered between 1-1000 Hz. The amplitude and noise level of evoked potentials were assessed utilizing a MATLAB algorithm incorporating a bandpass filter ranging from 0.1 to 300 Hz.

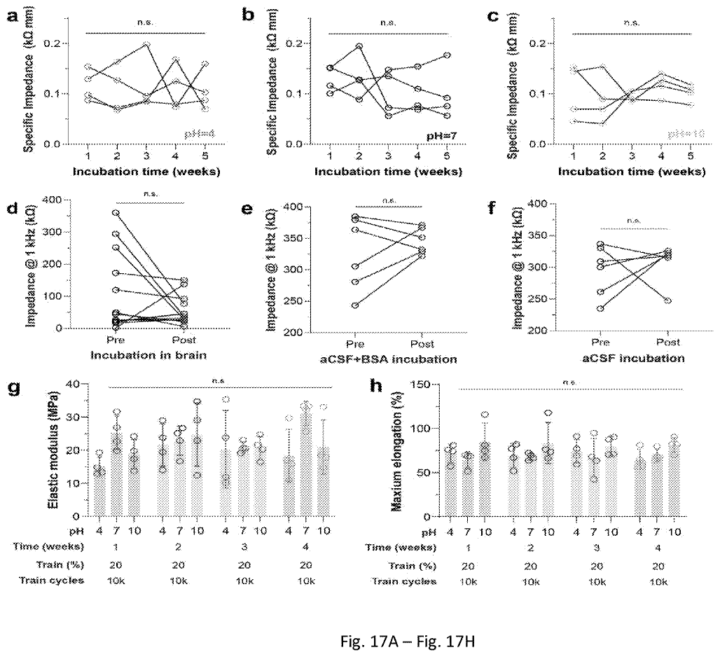

Stability of CNTs-PVA hydrogel fibers. To assess the electrical stability of CNTs-PVA hydrogel fibers, accelerated aging protocols²¹³ were employed, incubating trained CNTs-PVA fibers (0.16 wt. %, subjected to 5000 cycles at 20% strain) at 45° C. in artificial cerebrospinal fluid (aCSF) across varying pH conditions for a duration of five weeks (FIG. 17A-17C, 17F). By measuring specific impedance weekly from week 1 through week 5, no significant changes were observed, suggesting sustained electrochemical stability under accelerated aging conditions.

To investigate potential interactions with endogenous proteins, which could influence electrochemical properties, untrained CNTs-PVA fibers were incubated in mouse brain tissue (FIG. 17D) and in aCSF containing 1% BSA (FIG. 17E) for 14 days. Impedance values remained consistent pre- and post-incubation, indicating that the presence of proteins in physiological environments does not degrade the conductive performance of CNTs-PVA fibers.

Accelerated aging was used to determine its impact on the mechanical properties of the CNTs-PVA fibers. Fibers incubated at 45° C. for 4 weeks under different pH conditions (4, 7, and 10) were tested for changes in elastic modulus and stretchability. Using two-way ANOVA to assess statistical differences over time, both elasticity (FIG. 17G) and stretchability (FIG. 17H) remained stable throughout the four-week period. These results collectively demonstrate the durability of CNTs-PVA fibers under varying environmental conditions, underscoring their suitability for long-term in vivo applications where stable electrochemical and mechanical properties are critical.

Running wheel behavioral assay for freely moving EMG recordings. All the mice were acclimatized to a running wheel for 30 mins, 1 day prior to surgery. 3 days after the implantation of the TRAIN hydrogel electrodes, mice were connected to a DAM50 system through wires and acclimatized to the running wheel for 30 mins. The EMG data was filtered (100-3000 Hz) and collected through a recording system (PowerLab 4/20T, ADInstruments). Concurrently, a camera was used to record videos for the analysis of the gait, using DeepLabCut (DLC), of the mice during locomotion. Critical anatomical landmarks on the mice, encompassing the electronic pin, neck, back, iliac crest, hip, knee, ankle, front toe, and rear toe, were meticulously tracked during the mice's locomotion in the recorded videos. Using a custom-written MATLAB algorithm, a single gait cycle in variable-width runway tasks (VWRTs) was identified, defined by the two local minima observed in the plot of the distance between the fore toe and hind toe over time. This gait cycle was further segmented into swing and stance phases. From these data, kinematic stick diagrams representing the hindlimb movement during a single gait cycle were generated, providing detailed insights into the locomotor dynamics of the subjects.

Synchronized EMG and spinal cord electrophysiological recordings. Thy1-ChR2-EYFP mice were anesthetized using isoflurane (1.5% induction and continuously maintained at 1%) and the implanted 4-pin electronic device at the spinal cord was connected to a DAM8 system through wires. A TRAIN hydrogel electrode was inserted into the TA muscle in the hindlimb, a reference electrode was inserted into the right body of the mice, and a ground electrode was inserted subcutaneously into the tail. A 473 nm laser was used for transdermal optical stimulation over the hindlimb. Spinal cord electrophysiology and EMG data triggered by optogenetic activation were synchronized, filtered (10 Hz to 1 kHz), and collected through the DAM8 system.

Endogenous recordings in spinal cord under anesthesia. The spinal cord implanted mice were anesthetized using isoflurane (1.5% induction and continuously maintained at 0.5%). The 4-pin device was connected to a DAM50 system, and the spontaneous neural activities were filtered (100-3000 Hz) and collected through a recording system (PowerLab 4/20T, AD Instruments).

Open field behavioral assay for freely moving spinal cord recordings. Prior to recordings, all mice were acclimatized to an open field chamber for 30 minutes to minimize stress and adaptation effects. The same 4-pin device used in the anesthesia studies was connected to a DAM50 system for continuity in data collection. Spontaneous neural activities were filtered across a bandwidth of 100-3000 Hz and collected through the recording system (PowerLab 4/20T, AD Instrument). Concurrently, mouse behaviors were monitored via a camera strategically positioned above the open field chamber, allowing for the correlation of neural activity with observable behaviors.

### Example 2

### Hydrogel Fiber Optics

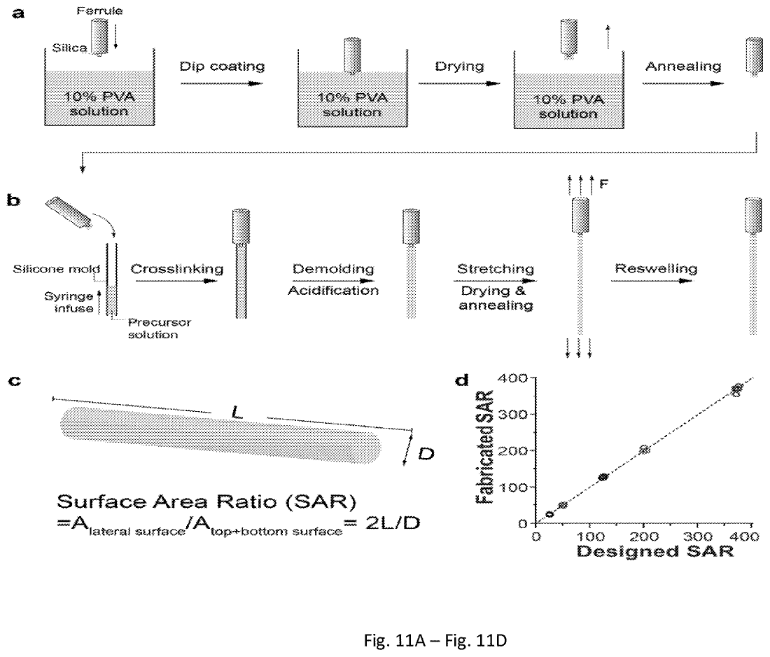

Hydrogel fibers were fabricated with smooth surfaces and high dimensional precision using molding and extrusion techniques. To quantify surface geometry, the surface area ratio is defined (SAR, FIG. 11C) as:

$$S A R = \frac{{A}_{lateral surface}}{{A}_{top + bottom surface}} = 2 L / D .$$

Using molds with varying SARs, a high correlation was observed between the designed and fabricated SAR values, with a linear fit (R²=0.9995, FIG. 3-1d), confirming the high precision of the molding and extrusion methods for producing hydrogel fibers with specific dimensions. This precise fabrication approach enables the customization of hydrogel fiber dimensions to meet specific application requirements.

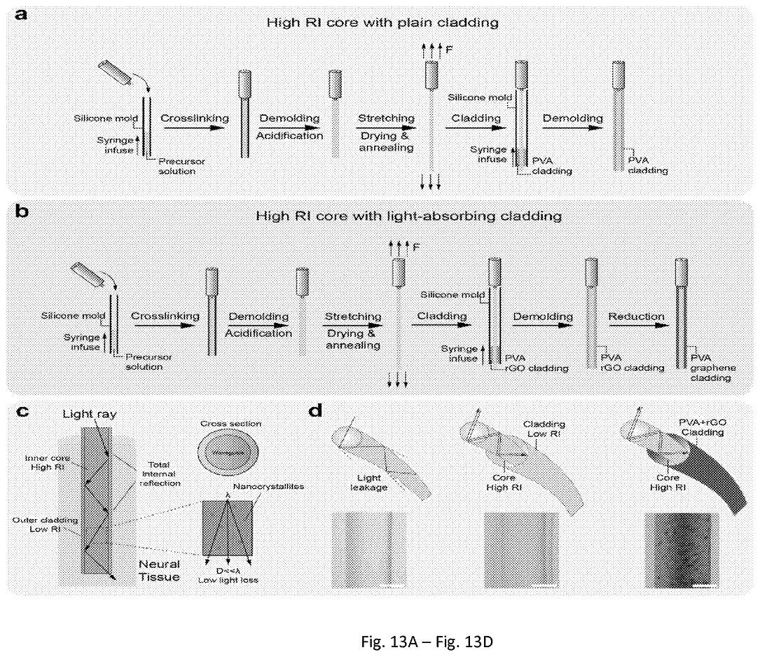

Total internal reflection (TIR) is the phenomenon in which waves arriving at the interface (boundary) from one medium to another (e.g., from water to air) (FIG. 13C) are not refracted into the second (“external”) medium, but completely reflected back into the first (“internal”) medium. It occurs when the second medium has a lower refractive index (RI) (or higher wave speed) than the first, and the waves are incident at a sufficiently oblique angle on the interface. Based on Snell's law, TIR can be described as follows: n₁ Sin_{θ}_{₁}=n₂ Sin_{θ}_{₂}, where n is the refractive index of mediums, and θ is the dihedral angles at the refractive interface. In order to achieve TIR, the arcsin function yielding θ_{c} is defined only if n₂≤n₁. Hence, for isotropic media, total internal reflection cannot occur if the second medium has a higher refractive index (lower normal velocity) than the first.

Light leaks through the interface when the refractive index of medium 1 (n₁) is less than or equal to that of medium 2 (n₂), and the angle of incidence is blow the critical angle, causing the refractive ray to exit the interface at an angle θ₂ relative to the normal.

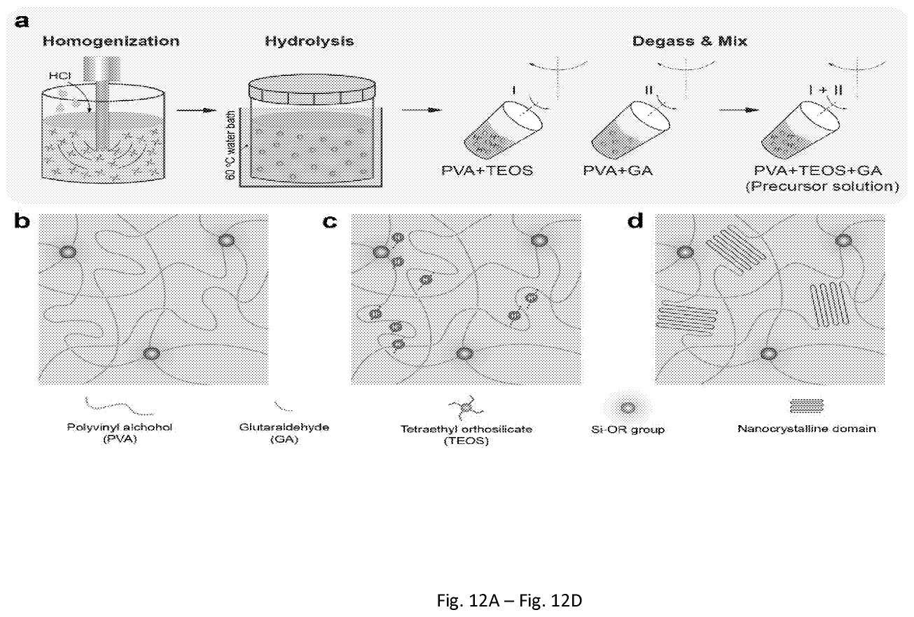

From the perspective of light transmission, current hydrogel fiber-optic devices face substantial light leakage at the core-cladding interfaces due to the inherently low refractive indices (RI) of fully hydrated hydrogels (~1.34), resulting in inadequate RI contrast between core and cladding materials. The RI of water is ~1.33 and the RI of body fluid is around ~1.34, when considering hydrogel optical fiber applications in biological bodies, to achieve TIR, the RI of hydrogel fibers must be higher than 1.34. To enhance the RI of PVA hydrogels, a fabrication strategy was developed that leverages controlled amorphous-to-crystalline transitions of metamorphic polymers, focusing on the following aspects: (i) folding and immobilization of polymer chains using multiple cross-linkers (FIG. 12A, 12B), (ii) modulation of intermolecular interactions within the hydrogel matrix (FIG. 12C), and (iii) induction of oriented growth of nanocrystalline domains (FIG. 12D). This strategy was implemented via three primary steps to control individual polymer chain folding, chain network interactions, and nanocrystalline growth.

Initially, TEOS hydrolysis was introduced within PVA solutions through a homogenization process (FIG. 12A), followed by the addition of GA as a general cross-linker. By employing dual cross-linkers, precise control over polymer chain mobility was achieved through covalent bonding, which allowed concurrent tuning of the hydrogel's RI. Subsequently, the cross-linked hydrogels were acidified to promote enhanced intermolecular interactions between polymer chains. Careful adjustment of the TEOS content enabled fine control over both the RI and light transmittance of the PVA hydrogel matrix. This approach has proven effective in overcoming traditional limitations in hydrogel fiber-optic devices, enhancing light transmission efficiency by optimizing the optical properties of the hydrogel core.

To exploit the tunable RI of the PVA hydrogels, hydrogel fibers were devloped featuring a core-cladding structure. The hydrogel core exhibits an RI of 1.396±0.004 in the fully hydrated state and 1.549±0.017 in the dried state, while the cladding has an RI of 1.346±0.001 (hydrated) and 1.492±0.045 (dried), ensuring total internal reflection (TIR) across varying hydration levels. Using identical molding and extrusion techniques, step-index hydrogel fibers were fabricated with a high-RI core and a low-RI plain cladding. In contrast to amorphous hydrogels, nanocrystalline hydrogels exhibit an elevated RI, attributable to reduced water content and densely packed nanocrystalline domains, which enhances light confinement within the fiber core^{150, 157}.

To further confine light within the hydrogel optical fibers, the cladding as engineered to be light-absorbing. A 37-μm-thick cladding layer composed of amorphous reduced graphene oxide (rGO)-PVA was developed. This design significantly mitigates light leakage by absorbing stray light at the core-cladding boundary, ensuring enhanced light retention within the fiber core and maximizing optical efficiency for in vivo applications.

FIGS. 13A-13D show step-index hydrogel fiber optic device fabrication with claddings. To fabricate a compact optical device with reduced graphene oxide (rGO) claddings, the same approaches were used as those used for assembling silica segments and ferrules to fabricate compact optical fibers (FIG. 13A). Firstly, the annealed ferrule-connected fiber was re-inserted into a silicone mold with a diameter of 500 μm. Next, GA was added into a 5 wt. % PVA solution and degassed the mixture. Similarly, hydrochloric acid (HCl) was added into another 5 wt. % PVA solution and degassed. The two solutions were then mixed with a graphene oxide solution (weight ratio of 1:1:1) and degassed. The resulting mixture of PVA-GA-HCl-rGO was infused into the 500 μm silicone mold and allowed to cross-link at room temperature for 4 hours, thereby forming a cladding layer around the optical fiber (FIG. 13C). To elute the core-cladding optical device, dichloromethane (DCM) was used. Subsequently, the compact optical device with the core-cladding was incubated in a 20 mL aqueous solution containing 1 g of sodium hydroxide and 0.3 g of sodium hydrosulfite at 40° C. for 1 minute to achieve a graphene cladding. Throughout the process, consistent mixing parameters were maintained using a planetary mixer at 2000 rpm for 1 minute under a vacuum of 16 kPa. FIG. 13C shows a schematic illustration of light transmission in a step-index hydrogel fiber. FIG. 13D shows a schematic illustrations and representative photographs of a PVA-TEOS-GA core hydrogel fiber, a PVA-TEOS-GA core-plain-cladding hydrogel fiber, and a PVA-TEOS-GA core-reduced reduced graphene oxide (rGO, 0.21 wt. %)-cladding fiber. Scale: 200 μm.

Fabrications of conductive hydrogel fibers. To prepare a 10 wt. % PVA solution, 10 g of PVA powder was combined with 90 g of MilliQ water, then heated at 100° C. with continuous stirring for 5 hours, yielding a clear, viscous solution. The CNTs paste was diluted using a 5 wt. % SDBS solution with a 1.6:1 ratio to mitigate CNT aggregation. To fabricate chemically cross-linked PVA-CNT hydrogel fibers, 100 μL of GA was added into 10 g of 10 wt. % PVA solution followed by mixing and degassing in a vacuum spinner (ARV310, Thinky). 150 μL of HCl was then added into 10 g of 10 wt. % PVA solution with mixing and degassing followed. Next, the diluted CNTs, PVA-GA, and PVA-HCl solutions were combined on a ratio of (0.6~1.4): 1:1 with the same mixing and degassing procedure. The homogeneous solution was then infused into silicone tubings and allowed to cross-link at room temperature (RT) for 2 hours. To elute the hydrogel fibers, DCM was used to swell the silicone tubings. The hydrogel fibers were then stretched (150% strain) and dried for 12 hours followed by annealing (100° C., 20 mins). The fibers were reswelled in PBS (1×) solution for later use.

Materials and reagents. Poly(vinyl) alcohol (PVA, 146,000 to 186,000 Da and 99+% hydrolyzed), Glutaraldehyde (GA, 25% in water), hydrochloric acid (HCl, 37%), Dichloromethane (DCM, 99.8%) were purchased from Sigma Aldrich. Carbon nanotubes (CNTs, 0.8%) were purchased from Ocsial. Sodium Dodecylbenzene Sulfonate (SDBS, 97%) was obtained from the Tokyo Chemical Industry. Silicon tubing (800 μm and 1 mm I.D.) was provided by MacMaster-Carr.

Dimension measurements of hydrogel fibers. Images of hydrogel fibers were captured using a microscope (AmScope) under a bright field while submerged in water. Each fiber was examined in three distinct areas, including both ends and the middle. Subsequently, the Image J software was used to measure the diameter of each fiber. In addition, the length of the fibers was measured using a caliper.

Mechanical training of conductive hydrogel materials. To mechanically train the as-fabricated conductive hydrogel materials, they were subjected to cyclic stretching tests (strain-controlled mode: 5%, 10% and 20% strain, and frequency of 0.5 Hz) for 5000 cycles using a horizontal mechanical testor (Univert, Cell Scale) in a water bath. A 4N load cell (Futek) was used. The conductive hydrogel materials were stretched with a constant strain amplitude, and the force variation was recorded over time.

Mechanical tests of conductive hydrogel fibers. Tensile test machine (Stable Micro System TA, XT plusC, 50N load cell) was employed to stretch the hydrogel fibers using tensile tests at a rate of 1 mm/s. The nominal stress was calculated using the formula σ=F/A, where F denotes the recorded force and A denotes the cross-sectional area of the fibers in the hydrated state. The strain was calculated using ϵ=ΔLL/, where ΔL represents the displacement, and L represents the gauge length. The elastic moduli (E) were determined by calculating the average slope of the stress-strain relationship in the first 10% of applied strain using linear regression. The maximum elongation (%) of the fibers was reported at the point of fracture in the stress-strain curve.

Electrochemical characterization of conductive hydrogel materials. Conductive hydrogel material impedance was systematically assessed using an electrochemical working station (Princeton Applied Research PARSTAT 2273). A 3-electrode electrochemical sink was used to characterize conductive hydrogel fibers with a sinusoidal driving voltage spanning a frequency range from 10 Hz to 1 MHz and an amplitude of 10 mV. The mechanically trained conductive hydrogel membranes were characterized by a customized device in three directions: parallel to the stretching direction (X), perpendicular to the stretching direction (Y and Z).

Impedance spectra of conductive hydrogel membranes were obtained with 2 platinum wires (distance 2 mm) across the directions that are parallel and perpendicular to the stretching direction. The impedance along with the membrane thickness was tested using 2 platinum sheets. The impedance of conductive hydrogel materials was normalized by the formula: R=1/A ρ, where R is the measured impedance, l is the length of the materials that were characterized, A is the area of the materials that were characterized, ρ is the specific impedance of the conductive hydrogel materials. The length of hydrogel film was measured by using a caliper and the thickness was tested via a micrometer.

X-ray scattering. X-ray scattering experiments were conducted using the SAXSLAB GANESHA 300XL instrument, equipped with a Dectris Pilatus 300K 2D CMOS photon counting detector (measuring 83.8×106.5 mm2). Wide angle X-ray (WAXS) measurements employed a 2 mm beamstop, and each sample was exposed for a duration of 300 s.

Measurement of crystallinities. The degree of crystallinity of hydrogel fibers and materials was assessed using a DSC instrument (2920 TA instrument). The PVA hydrogels were analyzed in the desiccated state. A small quantity of sample (1-15 mg) was loaded into a crucible (TA instrument T81006) and placed in a temperature-controlled DSC cell. A blank crucible served as a reference. The sample was heated from 30° C. to 300° C. in air, with a heating rate of 20° C./min. The differential heat flow to the sample and reference was recorded by the instrument. To determine the melting fusion enthalpy of endothermic peaks, heat flow (mW) over sample weight (mg) was plotted against time(s). The areas of melting endothermic peaks were integrated using TA analyze software (TA Universal Analysis). The degree of crystallinity a was estimated using the equation: α=ΔHf/ΔHm·100%, where ΔHf (J/g) was calculated from the integration of melting endothermic peaks and ΔHm (150 J/g) was the enthalpy of melting 100% of PVA crystallites.

Running wheel behavioral assay for freely moving EMG recording. All the mice were acclimatized to a running wheel for 30 mins, 1 day prior to surgery. 3 days after the implantation of the PVA-CNTs hydrogel electrodes, mice were connected to a DAM50 system through wires and acclimatized to the running wheel for 30 mins. The EMG data was filtered (100-300 Hz) and collected through the DAM50 system. Concurrently, a camera was used to record videos for the analysis of the gait, using DeepLabCut (DLC), of the mice during locomotion. Critical anatomical landmarks on the mice, encompassing the electronic pin, neck, back, iliac crest, hip, knee, ankle, front toe, and rear toe, were meticulously tracked during the mice's locomotion in the recorded videos. Furthermore, the mice's running speed was calculated by analyzing the trajectories of these identified landmarks. The methodology for determining running speed relied on the parabolic correlation between the distances separating the front and hind toes. This analysis was conducted using MATLAB, where local maxima were identified, corresponding to distinct individual running cycles.

Hydrogel synthesis. The chemicals used included tetraethyl orthosilicate (TEOS, Sigma-Aldrich 86578, 99%), hydrochloric acid (HCl, Sigma-Aldrich, 258148, 37%), glutaraldehyde solution (GA, Sigma-Aldrich G6257, 25% in water), and polyvinyl alcohol (PVA) with an average molecular weight of 146,000 to 186,000 Da and 99+% hydrolyzed (Sigma-Aldrich, 363065). Materials were used as received. MilliQ water with a resistivity of 18 MQ cm at 25° C. was used throughout the experiments. To prepare the PVA (10 wt. %) solution, PVA was dissolved in MilliQ water and stirred in a water bath at 100° C. for at least 4 hours until a clear and transparent solution was obtained. The hydrolysis of TEOS was carried out using HCl as a catalyst in PVA solutions with a molar ratio of TEOS:HCl:H2O=x: 4: y, where x was between 1 to 4, and y started from 4 to 16. TEOS solutions with concentrations ranging from 2 wt. % to 8 wt. % were added to the PVA solutions, which were then homogenized at two different levels. A mixture of HCl and MilliQ water in a molar ratio of 4:y, where y was in the range of 4 to 16, was added dropwise to the PVA-TEOS emulsion while homogenizing at 12000 rpm using a portable homogenizer until a stable emulsion was formed. The resulting emulsion was further homogenized using a high-speed homogenizer (FSH2A lab). The mixed solutions were stirred in a water bath at 100° C. for 1 hour until transparent solutions were obtained, followed by an additional 12 hours of stirring at 60° C. The composition of all solutions used is provided in Table 1.

TABLE 1
TEOS and PVA concentrations of PVA-TEOS solutions

| | TEOS | HCl | PVA |
|---|---|---|---|
| TEOS:HCl:H₂O | wt. % in PVA | wt. % in | wt. % in |
| (molar ratio) | pre-solutions | solutions | solutions |
| 1:4:4 | 2 | 0.014 | 10 |
| 2:4:8 | 4 | 0.014 | 10 |
| 3:4:12 | 6 | 0.014 | 10 |
| 4:4:16 | 8 | 0.014 | 10 |

Fabrication of hydrogel optical fibers. A step-index multimode silica fiber (core diameter 400 μm, NA 0.5, Thorlabs FP400URT) was prepared by removing the protective coating using a fiber stripping tool (Micro-strip, Micro Electronics, Inc). The stripped fiber was then divided into 13-mm segments using a diamond cutter. These fiber segments were inserted and extruded from one end of an optical ferrule (bore diameter 400 μm, Thorlabs CFX440-10) with a length of 2.5 mm and secured with EccoBond F adhesive (Loctite). Both ends of the silica fibers in the ferrules were polished using a polish kit (Thorlabs D50-F, NRS913A, and CTG913). The light transmission of all silica fibers and ferrules was tested by coupling with a 470 nm blue light-emitting diode (LED) (Thorlabs M470F3) after polishing. To remove the plastic coatings on the extruded silica fibers, they were treated with 2 M sodium hydroxide solution (Sigma-Aldrich, 1064980500) for 2 h followed by an additional treatment with chloroform (Sigma-Aldrich, 472476) for 30 min. A thin layer of 10 wt. % PVA was then coated on the extruded silica fibers via dip coating, and the PVA-coated silica fibers were air-dried at room temperature for 12 h and annealed at 100° C. for 2 h. A vacuum planetary mixer (Musashi ARV-310, 2000 rpm, and 16 kPa vacuum) was utilized for the mixing and degassing of all solutions. For degassing and mixing, 100 μL of GA was added to 10 g of 10 wt. % PVA pre-solution and agitated for 1 min. 10 g of pre-made PVA-TEOS solution was also degassed and mixed for 1 min. Subsequently, the above two solutions were combined (weight ratio of 1:1) and mixed for another minute. The resulting PVA-TEOS-GA solution was infused into silicone tubes (McMaster-Carr 5236k204, 80 mm in length), and the optic ferrules were inserted into the silicone tubes, with the silica fiber end connected to the PVA mixture. After curing at room temperature for 4 h, the PVA-TEOS-GA fibers were demolded using dichloromethane (DCM, Sigma-Aldrich, 270997, 99.8%) and washed with a large amount of water to remove residual chemicals for two days. Ferrule-connected fibers were air-dried at room temperature for 12 h and annealed at 100° C. for 20 min. Finally, the hydrogel fibers were rehydrated with MilliQ water before use. The compositions of all fabricated fibers are listed in Table 2.

TABLE 2
TEOS and PVA concentrations in PVA-TEOS-GA fibers

| | | TEOS | HCl | GA | PVA |
|---|---|---|---|---|---|
| | TEOS:HCl:H₂O | wt. | wt. | wt. | wt. |
| | (molar | % in | % in | % in | % in |
| Name | ratio) | fibers | fibers | fibers | fibers |
| 10P-1T-GA | 1:4:4 | 1 | 0.007 | 0.005 | 10 |
| 10P-2T-GA | 2:4:8 | 2 | 0.007 | 0.005 | 10 |
| 10P-3T-GA | 3:4:12 | 3 | 0.007 | 0.005 | 10 |
| 10P-1T-GA | 4:4:16 | 4 | 0.007 | 0.005 | 10 |

Core-cladding optical probe fabrication. A vacuum planetary mixer (Musashi ARV-310, 2000 rpm, and 16 kPa vacuum) was employed for mixing and degassing of all solutions. The optical fiber probes were first dried and then re-inserted into silicone tubing (McMaster-Carr 51845K66) and reswelled in water. For the preparation of the core-cladding optical fiber probes, 100 μL of GA was added to 10 g of 5 wt. % PVA pre-solution, which was then degassed and mixed for 1 minute. Additionally, 150 μL of HCl was added to 10 g of 5 wt. % PVA pre-solution, which was also degassed and mixed for 1 minute. The two solutions were combined (weight ratio of 1:1) and mixed for 1 minute. The resulting mixed solution was infused into the silicone tubing and allowed to cross-link for 4 hours at room temperature. The core-cladding optical fiber probes were extruded by immersing them in DCM and stored in MilliQ water until further use.

Measurements of R^{I} and transmittance. A series of hydrogel membranes were prepared via spin coating using a spin coating instrument (SETCAS, KW-4A) on silicon (Si) substrates (University Wafer, Inc., Model 447). The Si substrates were cut into square wafers (13.5 mm×17.5 mm) using a diamond cutter and then subjected to a rigorous cleaning process. The cleaning process involved washing and ultrasonication in Acetone (Sigma-Aldrich 179124, 99.5%) for 3 minutes, followed by rinsing with MilliQ water. The Si wafers were then washed and ultrasonicated in 30 wt. % H₂SO₄ solution (Fisher Chemical 210524, 95.0%) for 3 minutes, followed by rinsing with MilliQ water. Finally, the Si wafers were washed and ultrasonicated in 10 wt. % of H₂O₂ solution (Sigma-Aldrich 216763, 30 wt. % in water) for 3 minutes, followed by rinsing with 95% ethanol (Fisher Chemical A962P4, 95.0%). The Si wafers were mounted on the spin coater and coated with 10P-GA, 10P-1T-GA, 10P-2T-GA, 10P-3T-GA, and 10P-4T-GA membranes (n=4 for each group) at 1000 rpm for 10 s, and at 5000 rpm 50 s. PVA solutions used for the membranes were prepared using the same method as discussed above. After spin-coating, the PVA membrane-coated Si wafers were allowed to cross-link and dry in the air for at least 12 hours and then annealed at 100° C. for 20 minutes. The refractive index (RI) of the PVA membrane-coated Si wafers was measured using an ellipsometer (J.A. Woollam RC2) in the range of 400 nm to 700 nm. Measurements were carried out on the membranes in their desiccated states. A series of COMPACT hydrogel membranes (0-4 wt. % TEOS) were prepared using a similar procedure as described above but using a rectangular mold (21.5× 21.5× 1 mm). The membranes were demolded after cross-linking, dried at room temperature for 12 hours, and cut into small sheets (2×2 mm). The sheets were then annealed at 100° C. for 20 minutes and reswelled in MilliQ water for 1 hour. The RI of the membranes in their hydrated states was measured using a refractometer (Sper Scientific 300034) with water used for calibration.

Measurements of absorbance and fluorescence. A set of hydrogel membranes (designated as 10P-GA, 10P-1T-GA, 10P-2T-GA, 10P-3T-GA, and 10P-4T-GA, comprising 4 replicates for each group) were synthesized and cross-linked in a 96-well plate using established techniques. Subsequently, 1 mL of PVA solution was added to each well and allowed to cross-link and air dry for at least 12 hours, followed by annealing at 100° C. for 20 minutes. Rehydration of the membranes was achieved by the addition of 100 μL of MilliQ water to each well. To obtain transmittance spectra in the range of 400 nm to 700 nm, the 96-well plate was subjected to analysis using a plate reader (Biotek Synergy 2). Autofluorescence measurements were acquired using excitation/emission wavelengths of 470 nm/510 nm and 485 nm/520 nm, respectively. Membrane thickness was determined by caliper measurements and recorded three times to normalize the transmittance spectra and autofluorescence readings with respect to thickness. A blank control consisting of 200 μL of MilliQ water was included for comparison purposes.

Light attenuation evaluation. The light transmission loss of hydrogel fibers was tested by the cutback method. Ferrule-connected hydrogel fibers were inserted into a plastic tube (5 cm in length and 3 mm in diameter) and injected with 1 wt. % agar gel to maintain their hydrated state. The ferrule was connected to a 470 nm LED light (Thorlabs M470F3) via an adaptor (Thorlabs SMIFCM). The power (in dB) of transmitted light through the hydrogel fiber was measured using a power meter (Thorlabs, PM16-122). The original power reading was recorded, and a 5 mm interval of cutting was adapted. Starting from the far end of the ferrule, the output power was measured after each cut using a cutter. The attenuation coefficient (a) was calculated using the formula.

$$α = ( \frac{1 {0}^{4}}{{L}_{1} - {L}_{2}} ) \cdot log ( \frac{{P}_{1}}{{P}_{2}} ) ,$$

where L₁ and L₂ represent the original and cut lengths of the fiber in meters, respectively. P₁ and P₂ are the transmitted power readings before and after the cut, respectively.

Tensile tests. To ensure consistency, all hydrogel fibers were hydrated prior to the extension test. Tensile tests were conducted using a tensile instrument equipped with a 50N load cell (Stable Micro System TA, XT plusC). The fibers were stretched at a constant rate of 1 mm/second. The nominal stress was calculated from the formula:

$$σ = \frac{F}{A} ,$$

where F represents the force measured by the instrument, and A represents the cross-sectional area of the fibers in their hydrated state. The strain was calculated using:

$$ε = \frac{Δ L}{L} ,$$

where ΔL represents the displacement and L represents the initial gauge length. Two marks were labeled on the fibers using a sharpie pen to determine the initial gauge length L prior to the tensile test. A high-resolution camera was used to capture the entire tensile process and track displacement. The stress-strain curve was generated based on the calculated nominal stress and strain. The elastic moduli € were determined by calculating the average slope of the stress-strain relationship in the first 10% of applied strain. The average slope was determined by linear regression analysis (OriginLab Corporation). The stretchability of the fibers was reported as a percentage of the strain at the fracture point obtained from the stress-strain curves. The bending stiffness of hydrogel and silica fibers was measured using a mechanical tester (CellScale, Univert) equipped with a three-point beam bending setup. A deflection amplitude of 100 μm within the frequency range of 0.5-4 Hz (heartbeat frequency range) was employed^{2, 3}.

Beam bending tests. The bending stiffness was measured with a dynamic mechanical analyzer (Q800, TA Instruments and Advantage v5.5). Different samples (n=3 fibers) of 1.2 cm lengths were mounted in a single cantilever clamp and tested with a frequency sweep (0.1-10 Hz) under controlled displacement of 20 μm at 37° C. The cyclic bending tests (n=3 fibers) for soft gut fibers was performed with mechanical testing machine (Z2.5 with testXpert Ill VI.11, Zwick/Roell) over 104 cycles, while recording the light output from μLEDs after every decade. The diode based thermal sensors were calibrated against a commercial thermocouple (NeuLog, NUL-203) by equilibrating the fiber and thermocouple in a hot water-bath or a hot plate at different temperatures and recording the current response from the diode at 2.2 V forward bias (n=4 devices).

X-ray diffraction. X-ray scattering measurements were conducted using the SAXSLAB GANESHA 300XL instrument, equipped with a Dectris Pilatus 300K 2D CMOS photon counting detector (size: 83.8×106.5 mm²). A small-angle 2 mm beamstop was utilized for SAXS measurements, while a wide-angle 2 mm beamstop was employed for WAXS measurements¹. The exposure time was set at 600 seconds. The average size of the nanocrystalline domain was determined using Scherrer's equation, which is expressed as:

$$D = \frac{k λ}{βcosθ} ,$$

where k is a dimensionless shape factor that varies based on the actual shape of the nanocrystalline domain (k=1, approximating the spherical shape of the nanocrystalline domains), λ is the wavelength of X-ray diffraction (λ=1.54 Å), θ is the peak of the Bragg angle, and β is the full width at half maximum (FWHM) of the WAXS peaks. The d-spacing between nanocrystalline domains was calculated using:

$$d = \frac{2 π}{{q}_{max}} ,$$

where qₘₐₓ is the q value at its maximum intensity from SAXS patterns. The FWHM (β) and qₘₐₓ were obtained by curve fitting of the WAXS and SAXS patterns, respectively, in Origin software (OriginLab Corporation). For representative WAXS and SAXS 2D patterns, each hydrogel fiber was repeated three times with similar results.

Differential scanning calorimetry. The degree of crystallinity of hydrogel fibers and materials was assessed using a DSC instrument (2920 TA instrument). The PVA hydrogels were analyzed in the desiccated state. A small quantity of sample (1-15 mg) was loaded into a crucible (TA instrument T81006) and inserted into a temperature-controlled DSC cell. A blank crucible served as a reference. The sample was heated from 30° C. to 300° C. in air, with a heating rate of 20° C./min. The differential heat flow to the sample and reference was recorded by the instrument. To determine the melting fusion enthalpy of endothermic peaks, heat flow (mW) over sample weight (mg) was plotted against time(s). The areas of melting endothermic peaks were integrated using TA analyze software (TA Universal Analysis). The degree of crystallinity α was estimated using the equation: α=(ΔH_m)/(ΔH_m). 100%, where ΔH_m (J/g) was calculated from the integration of melting endothermic peaks and ΔH_m (150 J/g) was the enthalpy of melting 100% of PVA crystallites. For representative DSC data, each sample was repeated three times with similar results.

The fabricated COMPACT hydrogel fibers (3 wt. % TEOS) were incubated at 37° C. under physiological-like solutions (saline, ionic strength 305~310 mOsm, pH from 6.0 to 8.0) over 3 months to validate the stability of hydrogel materials. The dimensions of fiber were measured before and after the incubation and statistical analysis was performed on the dimensions between pre-incubation and post-incubation each week.

Step index optical fibers with tunable optical properties. Considering fiber optics in vivo applications, the optical, mechanical, and cytotoxicity properties of PVA hydrogel optical fibers were examined. To ensure efficient light transmission for optical stimulation and recordings, two important parameters of the hydrogel fiber core were considered: RI and light transmittance. The RI of PVA hydrogels across varying PVA concentrations was examined, observing an increase in RI with higher PVA content. Additionally, the findings indicate that the RI of these hydrogels can be effectively modulated by adjusting the content of tetraethyl orthosilicate (TEOS). Specifically, PVA hydrogels containing 0 wt. % to 4 wt. % TEOS demonstrated refractive indices from 1.37 to 1.40 in their hydrated state and from 1.48 to 1.60 in their desiccated state (aligning with RI values reported for other conventional polymeric materials¹⁵⁹.

Although all the transmittance remained above 96%, increasing TEOS content also led to decreased transmittance, and increased absorbance and autofluorescence (17.8% increase of 4 wt. % TEOS hydrogels compared to 0 wt. % TEOS hydrogels, excitation wavelength: 485 nm, excitation peak: 520 nm. The optimal TEOS content was chosen as 3 wt. %, which resulted in hydrogels with 1.54±0.01 of refractive index, >96% of transmittance (for 0.15±0.02 mm thick membranes), and 6.13±0.16 relative fluorescent units (RFU)/mm of autofluorescence (for 0.15±0.02 mm thick membranes. water: 3.70 RFU/mm).

Next, PVA hydrogel optical fibers were first fabricated into step-index optical fibers (FIG. 13A). Increased RI contrast between optical core and cladding layers ensures light transmission with a wider numerical aperture. Based on tunable refractive indices of the PVA-TEOS-GA hydrogels, step-index hydrogel fibers with high-RI core (n_{core}=1.40) and low-RI cladding (n_{cladding}=1.34) were designed. The function of RI-contrasting core-cladding structures was validated by comparing the light transmission between bare core fibers, step-index fibers with plain cladding and those with light-protective cladding. The bare core fibers (diameter of 329±17 μm) exhibited a relatively high attenuation (1.87±0.53 dB/cm) while introducing a thin low-RI cladding layer (thickness of 84±4 μm on the surface of 372±10 μm cores, n_{cladding}=1.34) decreased the light transmission attenuation to 1.75±0.08 dB/cm. A representative light-absorption nanomaterials¹⁶⁰, reduced graphene oxide (rGO) was loaded into low-RI cladding to further protect light leakage from fibers' lateral surface and consequently reduced the light attenuation to 0.94±0.25 dB/cm (core 339±35 μm, cladding: 36±11 μm of 5 wt. % PVA with 0.21 wt. % rGO) (FIG. 13B).

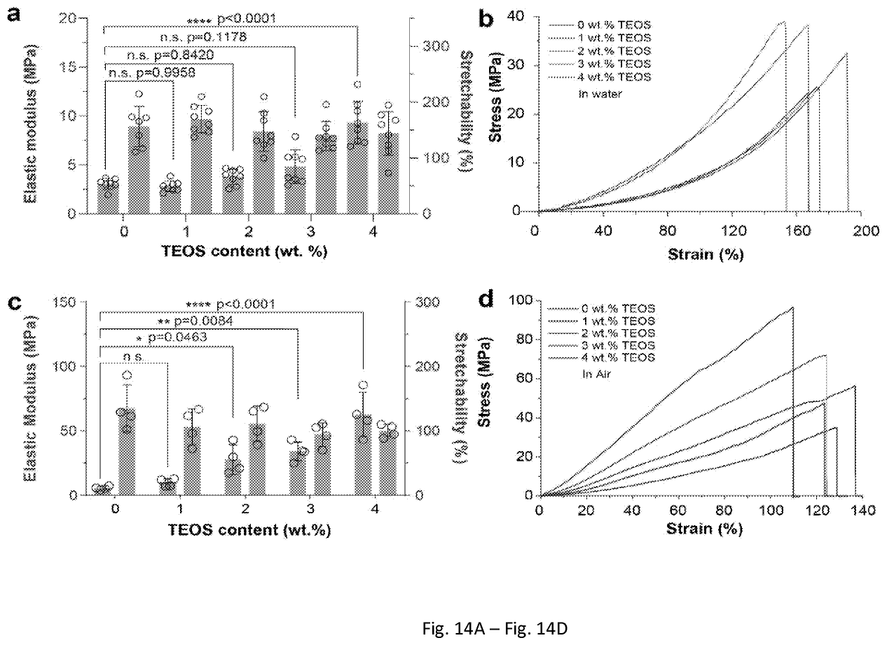

To simulate in vivo conditions, the mechanical properties of PVA hydrogels in their hydrated state were assessed. PVA hydrogel fibers displayed relatively low elastic moduli while retaining substantial stretchability (FIGS. 14A-14D). The optimized PVA hydrogel fiber formulation (3 wt. % TEOS, 12 mM HCl acidification treatment, and 200% pre-stretch, diameter: 227±18 μm) exhibited an elastic modulus of 4.8±1.7 MPa and stretchability of 139.4±26.0% in the hydrated state (FIGS. 14A, 14B).

When compared to tensile test results in ambient conditions, the optimized PVA hydrogel fiber (3 wt. % TEOS, 12 mM HCl acidification treatment, and 200% pre-stretch) maintained an elastic modulus of 27.73±11.39 MPa and stretchability of 111.21±27.32% (FIGS. 14C, 14D). Notably, the modulus decreases upon hydration due to water absorption, facilitating implantation into nervous tissue without the use of guides. Once implanted, the fiber's modulus is further reduced as it absorbs bodily fluids, enhancing less damage to the neural tissues.

Although the PVA optical hydrogel fibers exhibit an elevated elastic modulus, a comparison with state-of-the-art hydrogel interfaces reveals that the fibers maintain a relatively high-water content and significantly lower elastic modulus. This characteristic effectively reduces the mechanical mismatch between the interface and neural tissues, promoting a more compliant and biocompatible interface for neural applications. Various hydrogel polymers may be employed to match the elastic modulus of Ionic hydrogel¹⁶², Peripheral nerve¹⁶³, Heart¹⁶³, Skin¹⁶³, Tendon^{163, 164} e.g., poly(3,4-ethylenedioxythiophene), polystyrene sulfonate (PEDOT:PSS)¹⁶¹ Polydimethylsiloxane (PDMS)^{83, 165-168}, Styrene-Ethylene-Butylene-Styrene (SEBS)¹⁶⁹, Poly(1,8-octanediol-co-citric acid) (POC)¹⁷⁰, Polyimide (PI)^{154, 171}, Polycarbonate (PC) and Cyclic olefin copolymer (COC)^{54, 64, 172}, Poly(etherimide) (PEI)⁵⁴, (epoxy-based negative photoresist) SU-8171, and Poly(glycolic acid) (PGA)^{173, 174}.

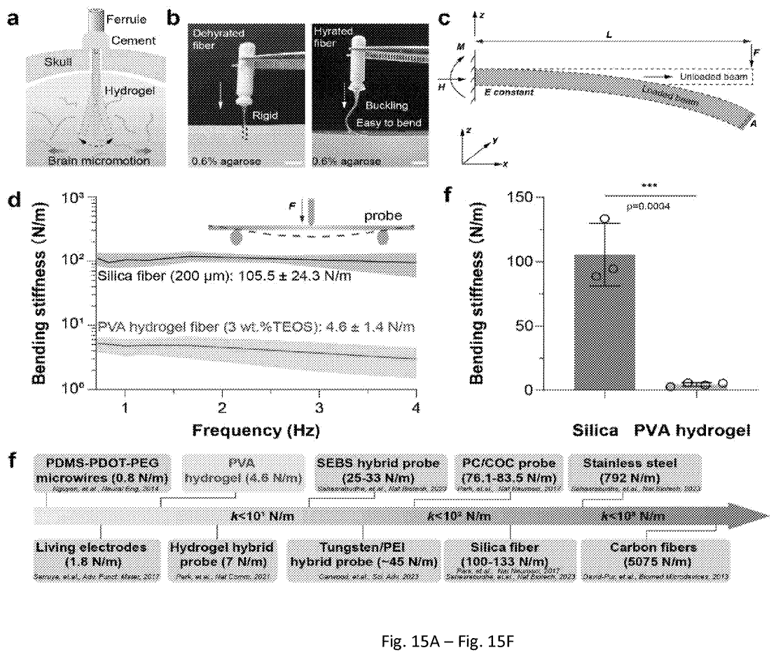

In the context of tissue micromotion¹⁷⁵, it was hypothesized that the bending stiffness is more relevant than the elastic modulus, since the probes are expected to be fixed to the skull and are not anticipated to experience compressive or tensile stress. Although the probes are flexible and maintain their functions under significant deformations, a mechanical analyzer in a 3-point beam bending mode was used to quantify the bending stiffness of PVA hydrogel fibers and commercialized silica optical fibers in the frequency range of mammalian locomotion, respiration and heartbeat (0.05-4 Hz, FIG. 15D, 15F)¹⁷⁶. PVA hydrogel fibers were found to exhibit bending stiffness values of 4.6±1.4 N/m. Compared to silica fibers (~20 GPa elastic modulus)¹⁷⁷ and polymer fibers (~1 GPa elastic modulus)⁶⁴, PVA hydrogel fibers offer improved mechanical matching to the nervous tissues (1-4 kPa)⁹¹ and much lower axial bending stiffness (FIGS. 15A-15F) to achieve less neural tissue damage from micro-motion involved in vivo studies¹⁷⁸. The bending stiffness of the PVA hydrogel optical probes was compared with that of other advanced optical and multifunctional probes. The PVA hydrogel probes exhibited a markedly lower bending stiffness, highlighting their potential for enhanced flexibility and reduced mechanical mismatch in biological applications.

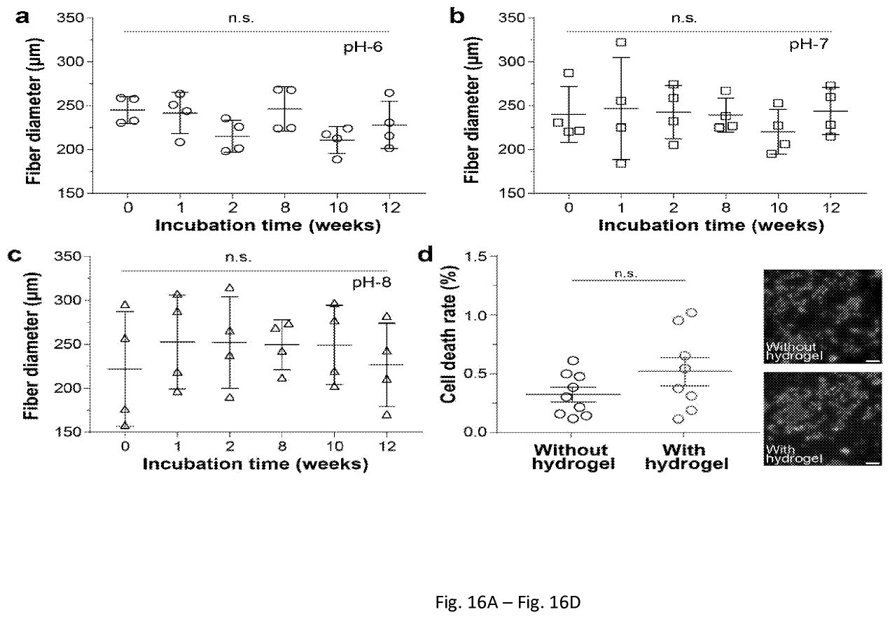

The effect of crystallization-enabled size reduction of PVA hydrogels was investigated for its ability to counteract the intrinsic swelling that typically occurs upon hydration, thereby maintaining structural stability under in vivo conditions. Hydrogel fibers were incubated in ex vivo physiological conditions (pH 6-8, 37° C., saline solution), and dimensional stability was monitored over time. Fiber diameters remained stable for up to three months across varying pH levels (FIGS. 16A-16C), indicating that the optical fibers offer long-term structural integrity with minimal mechanical mismatch.

To assess cytotoxicity, PVA hydrogel fibers were incubated in cell culture medium for 48 hours at 37° C., followed by the exposure of HEK 293T cells to the preconditioned cell culture medium for an additional 48 hours. Cell viability was assessed using Calcein-AM (green) to label viable cells and homodimer-1 (red) to label non-viable cells. Results showed no significant difference in viability between cells exposed to PVA hydrogel fibers and control cells (FIG. 16D), supporting the non-cytotoxic nature of the PVA hydrogels, consistent with previous findings on their biocompatibility¹⁸⁴.

Biological testing. HEK 293T cells were authenticated before receiving. HEK 293T cells were maintained in DMEM (with GlutaMax, Sigma Aldrich, D5796)+10% fetal bovine serum and seeded in a 24-well plate. COMPACT hydrogel fibers (3 wt. % TEOS) were incubated in DMEM for 24 hours at 37° C. Hydrogel-incubated DMEM was then added to the well plate and incubated for 24 hours. Calcein-AM (green, 2 μL of 1 mg/mL per well, Sigma-Aldrich 17783) was added to indicate living cells, and ethidium homodimer-1 (red, 2 μL of 1 mg/mL per well, Sigma-Aldrich 46043) was added to indicate dead cells. A fluorescent microscope (Nikon TiU with SOLA Light Engine Gen III illumination hardware and PCO panda sCMOS camera) was used to take images of cells with and without hydrogel incubation. Image J was utilized to count living cells and dead cells. Cell death rate (%) was calculated by using the formula:

$$death rate ( \% ) = \frac{dead cell numbers}{total cell numbers} \cdot 100 \% .$$

Following a four-week recovery period, hSyn::GCaMP6s injected mice were tethered to a fiber photometry (FIP) system (RWD and Thorlabs. Inc) using silica fiber (with a core diameter of 400 μm and a numerical aperture of 0.5, Thorlabs FP400URT). The silica fiber was connected to the FIP system using an adaptor (Thorlabs SMISMA), and a ferrule (Thorlabs CF440) was fixed to the other end of the fiber. The ferrule was coupled to the implanted fiber probe using a connecting sleeve (Thorlabs ADAF1). The mice were placed in a custom-made chamber (20×20×20 cm) for social preference tests, and fluorescent signals were computed using custom-written Python code. To excite the fluorescent signal, a custom setup consisting of a 470 nm LED (Thorlabs M470F3), a 405 nm LED (Thorlabs M405F3), and dichroic mirrors (Thorlabs DMLP425R) were used. Illumination periods were determined by detecting synchronization ON/OFF pulses for each LED, with each illumination containing pulses at 10 Hz. To eliminate moving artifacts, the fitted 470 nm signals were subtracted from the fitted 405 nm signals.

For all behavioral experiments, adult C57BL/6 mice implanted with optical fiber probes were utilized during the dark phase of the light/dark cycle and were given at least 30 minutes of acclimatization in the behavior chamber before testing. Adult male C57BL/6 mice aged 5-6 weeks were used as strangers, and tests were performed in a dark environment. A chamber box (20×20×20 cm) containing a social cage was utilized for social interactions. Subsequently, a strange mouse was introduced to the social zone, and the test mouse was exposed to the strange mouse and allowed to interact freely. Concurrently, GCaMP fluorescence changes were recorded during social tests. A dark-vision camera was installed above the social chamber to record video footage during the social tests. The time spent interacting and the distance of social interaction were analyzed using customized algorithms for social interaction assessment with DeepLabCut. The analyzed social interaction epochs were then correlated with GCaMP signals.

The mice were euthanized using fatal plus (Vortech Pharmaceuticals, LTD) and transcardiac perfusion was carried out using 20 mL of PBS (Sigma-Aldrich P3813) solution followed by 20 mL of 4% paraformaldehyde (PFA, Sigma-Aldrich 8187151000) solution. The brains were then dissected from the bodies and fixed in 4% PFA solution at 4° C. overnight. After fixation, the brain tissues were treated with 30% sucrose in PBS for 2 days and subsequently frozen at −20° C. in an O.C.T. cube (21.5×21.5×22 mm) and sectioned on a cryostat (Leica CM1900) with a thickness of 20 μm. The sectioned tissues were then permeabilized with PBST (0.3% Triton-X-100 in PBS, Sigma-Aldrich 93443) for 15 minutes at room temperature and blocked with 1% bovine serum albumin in PBS (Sigma-Aldrich A9647) for 30 minutes prior to staining. Primary antibody solutions were applied to stain the tissues and incubated overnight at room temperature. After washing the tissues with PBS three times, secondary antibody solutions were applied and incubated at room temperature for 2 hours. The tissues were then washed with PBS three times and mounted on glass slides. DAPI mounting medium (Southernbiotech, Fluoromount-G, Cat. No. 0100-01) was used to mount the cover glass on top of the glass slide with the sections. The slides were left to dry in the air at room temperature overnight before images were acquired using a confocal microscope (Leica SP2 and ZEISS LSM 880, confocal fluorescent imaging was repeated 10-30 times with similar results). The quantitative analysis (fluorescence intensity, total fluorescence area and total cell number counts) was performed with ImageJ. An area analysis of antibody labeled cells was performed by creating binary layers of the implantation sites using the threshold tool and quantified using the measurement tool in Image J⁴.

Statistical analyses were conducted using GraphPad Prism version 10 and R Studio. Sample sizes were based on pilot and previously (similar types of) experiments and were not statistically predetermined. Initially, the Shapiro-Wilk test was applied to assess data normality. The data in all main figures met the normality criteria, enabling us to use parametric statistical tests. For comparing three or more independent groups, one-way Analysis of Variance (ANOVA) with Tukey's Honest Significant Difference (HSD) test applied for post-hoc multiple comparisons. To analyze unpaired data across two groups, the unpaired student's t-test was used. Similarly, the paired student's t-test for paired data sets was applied. P-values less than 0.05 were considered as significant. The specific significance levels are as follows: * for 0.01≤P<0.05, ** for 0.001≤P<0.01, *** for 0.0001≤P<0.001, and **** for P<0.0001.

Unlike rigid silica optical fibers, the modulus and dimensions of PVA hydrogel optical fibers change upon implantation into biological tissues, as body fluids infiltrate the hydrogel matrix. This change in stiffness was leveraged by implanting PVA fibers in their dried state to facilitate precise targeting of brain regions. However, calibration of dimensional changes is necessary for accurate positioning. To simulate brain tissue, 1% agarose gel was used as a phantom and the diameter and length of the fiber probe measured at various stages: after reswelling in water, reswelling in 1% agarose gel, and in the dried state. Comparisons of fiber length and diameter between the water- and agarose-reswollen states reveal a linear relationship in both length (slope 1.195) and diameter (1.141). These linear correlations serve as offset factors to adjust coordinates when PVA fibers are implanted in the dried state.

Fiber photometry recordings via PVA hydrogel optical probes concurrent with social interaction in mice. To validate their functionality for in vivo optical interrogation, the PVA hydrogel fibers were tested with photometric recording in the context of mouse social behaviors. Activation of VTA region and its related circuits has been studied with various techniques 185 related to social behaviors in mice. As a proof-of-concept application, PVA hydrogel fibers were validated to photometrically record mouse deep brain structure, VTA, with concurrent social behavior observation. PVA optical fibers (580±35 μm) were unilaterally implanted in VTA after injecting AAV containing genetically encoded calcium indicator (hSyn::GCaMP6s). A fiber photometry system (wavelengths: λ_{isosbestic point}=405 nm, λ_{excitation}=470 nm, λₑₘᵢₛₛᵢₒₙ=510 nm) was used to collect GCaMP fluorescent changes as proxies to reflect the neural activity^{35, 41}. After an incubation period of 4 weeks for AAV expression, mice were subjected to social behavioral tests with concurrent photometric recordings. Mouse social interactions were analyzed with DeepLabCut (DLC) markless pose estimation and a custom developed MatLab algorithm¹⁸⁶. The increased fluorescent intensity of GCaMP was correlated with mouse social interaction epochs. Linking neural activities at the cellular level to system neuroscience behavioral assessment provides important tools to discover causal relationships of neural circuits and behaviors for neuroscience studies.

The initial assessment targeted the immune response within brain tissues at 14 days following implantation. Findings revealed a notably diminished presence of astrocytes, microglial aggregates, activated macrophages, and IgG surrounding the hydrogel fiber implantation sites compared to the more rigid silica fibers. This outcome suggests that the hydrogel fibers, with their softer, more compliant structure, may promote a more biocompatible environment by minimizing the activation of neuroimmune cells.

The observed decrease in astrocytic and microglial reactivity around hydrogel fibers likely indicates a milder acute glial response, which is critical in the early stages of reducing inflammation and preventing the rapid onset of scar formation. In this acute 14-day post-implantation period, the reduced macrophage activation and lower IgG levels near the hydrogel fibers suggest that softer materials can modulate the initial immune response, potentially limiting the cascade of inflammatory signaling pathways that typically follow implantation. This attenuation of the acute immune response is advantageous in preserving local cellular environments and minimizing early structural disruption in the tissue, both of which are essential for establishing a favorable foundation for the longer-term stability and functional integration of neural implants.

Subsequently, the immune response at 30 days post-implantation was evaluated, still within the acute phase, and a lower incidence of astrocyte and microglial reactivity observed around the hydrogel fiber sites compared to those with silica fibers. In aligned with the PVA hydrogel optical fibers having a much lower bending stiffness compared to silica fibers, this reduced cellular response in the early post-implantation period highlights the potential of hydrogel fibers to mitigate the initial inflammatory reaction, which could be crucial for minimizing early-stage glial scarring and supporting better integration of the implant within neural tissue.

Unlike traditional methods that stabilize hydrogels through desiccation, where polymer chain collapse during drying leads to reversible swelling upon rehydration, the PVA-TEOS-GA hydrogels demonstrate a unique stability in the hydrated state due to their nanocrystalline polymer structure and enhanced interpolymer chain interactions. This configuration maintains stable folding, resulting in a robust structural integrity even under continuous physiological conditions. Over a 3-month incubation at physiological temperature and osmolarity, the PVA hydrogel fibers retained their intended diameters with a variance of less than 1% (FIGS. 16A-16D), underscoring their dimensional stability in miniaturized form for in vivo applications.

In comparison to conventional high-water-content hydrogel optical fibers, which have a modulus in the kPa range and an inherently low RI due to water's RI (~1.33), the hydrogel fibers achieve a balance of mechanical and optical properties optimal for neural interfacing. The low elastic modulus and bending stiffness of these PVA hydrogel fibers enable minimally invasive, long-term optical recordings in vivo by reducing tissue damage while maintaining high light transmission efficiency.

Historically, fiber photometry has relied on commercially available silica fibers due to their high RI and light-guiding efficiency¹⁸⁷. However, the rigidity of silica fibers limits their applicability in delicate neural regions, including the deep brain and complex structures like the spinal cord, as their stiffness can cause mechanical disruption to sensitive tissues, the PVA hydrogel optical fibers present a promising alternative, offering a low modulus and high RI, thus enabling deep brain interrogation with minimal tissue disruption. This advancement not only facilitates neural recordings in deep and complex brain areas but also supports applications in freely moving animals, allowing for in-depth studies of naturalistic behaviors, such as social interactions in mice. This approach opens new avenues for neuroscience research by combining the benefits of soft, flexible materials with high-performance optical properties in challenging in vivo environments.

Soft-material solutions have advanced motion-adaptive interfaces to enable neural modulation and recording in vivo, particularly within tissues experiencing complex mechanical dynamics, such as the spinal cord¹⁸⁸⁻¹⁹¹ and peripheral nerves^{150, 192}. These nerve tissues often suffer from damage due to mechanical mismatch between traditional rigid implants and the delicate nervous system, hindering access to deep structures under freely moving behavioral contexts. Therefore, a motion-adaptive soft material neural interface is required to minimize tissue damage during animal natural movement and achieve optimal recording results from physiological signals.

Engineering approaches have been adopted to enhance the electrical functionality of these interfaces. Fractal design concepts provide the capacity to extend conventional silicon-based technologies with soft materials and curvilinear structures¹⁹³. Conductive coatings¹³⁸, such as conductive polymers^{139, 192, 251} and micro-cracked metallic films^{45, 194} applied to soft and elastic substrates, have shown substantial success in electrical stimulation and recording in spinal cord tissue and the peripheral nervous system. The soft and elastic polymer matrix ensures flexibility and stretchability, while the conductive pathways, formed either through the conjugation of polymer backbone or metallic nano- and micro-structures, maintain conductivity under strain. However, most epidural electronics are designed to interface with the surface of the spinal cord, featuring large areas of flexible surface electrodes but lacking access to single-unit neural recordings, especially in the deep ventral structures. Due to the complex anatomical structures and mechanical conditions in the spinal cord, especially in awake and moving animals, the surface coating designs are also limited by the risk of delamination and fatigue under large strains over long-term implantation.

Penetrating microelectrodes were hypothesized to access deep tissues with greater spatial precision for intraspinal recordings than surface epidural electrodes. To achieve stable electrical properties under stretching, embedding conductive fillers directly into the polymeric matrix can create robust, electrically percolative pathways^{195, 252}. Particularly, nanocomposite materials containing high-aspect-ratio conductive fillers, allow for the reconstruction of percolation networks through the organization of nanofillers¹³⁷. Such methods address the risk of technical failures of delamination and the diminished overlap of percolation pathways under repeated stretching. Hydrogels show considerable promise as an elastic soft substrate due to their tissue-like mechanical properties (8 kPa-5 MPa)¹⁹⁶ and biocompatibility. Semi-crystalline polymers-based hydrogels, such as cross-linked polyvinyl alcohol (PVA) hydrogel, contain both amorphous polymer chains and polymeric nanocrystalline domains and offer a low elastic modulus and high fatigue resistance under complex mechanical conditions^{115, 150}. The integration of conductive nano-fillers within the hydrogel matrix intertwines with polymer chains to create three-dimensional percolative pathways within the elastic substrates.

Both computational and experimental studies have demonstrated that the conductive pathways of nanofillers can be regulated and reconstructed through stretch-induced kinematic movements and self-organization^{197, 198} Additionally, mechanical stretching can induce nanocrystalline re-alignment in hydrogels¹¹⁵. In a nanocomposite design, polymeric nanocrystalline support enhances durability and stretchability, while the anisotropic conductive pathways of nanofillers ensure the conductivity necessary for collecting in vivo electrical signals under complex tissue movements. Due to the chemically inert property and high-aspect-ratio structures, carbon-based conductive fillers are particularly suitable for in vivo physiological metabolic environments and the formation of conductive pathways with low concentrations.

Inspired by stretch-induced polymeric nanocrystalline growth and nanofillers reconstruction, carbon nanotubes (CNTs) were introduced into fatigue-resistant PVA hydrogels to create a set of conductive CNTs-PVA hydrogel fibers for electrical recording in vivo. A decrease in hydrogel fibers' impedances was observed after applying cyclic mechanical stretching (5,000 cycles at different strains: 5%, 10%, and 20%) and anisotropic conductivity improvement aligned with the stretching direction after cyclic stretching. Electron microscopy, stimulated Raman scattering microscopy and X-ray examination revealed a Tension Reinforcement for AnIsotropic Nano-orientation (TRAIN) mechanisms underlying the directional conductivity improvement (dispersion degree of CNTs in PVA matrix: 9.60±2.22°). Representative CNTs-PVA hydrogel fibers maintained 64.5±7.9% stretchability and 900±149 kΩ (@ 1 kHz) with a diameter of 187±13 μm. For in vivo testing, the CNTs-PVA hydrogel fibers were fabricated into electronic devices and implanted them into mouse hindlimb muscles and spinal cords. In muscle-implanted transgenic Thy1-ChR2-EYFP mice, light-evoked muscle responses were triggered and captured electromyographic (EMG) signals through transdermal stimulation with blue light (473 nm) under anesthetized conditions. Moreover, when implanted in the tibialis anterior (TA) and gastrocnemius soleus (GS) muscles, the hydrogel microelectrodes facilitated simultaneous EMG recordings from both muscles during voluntary wheel running tests. With the miniaturized device involving hydrogel microelectrodes being implanted in the spinal cord and hindlimb muscles, the activities of ventral spinal cord neurons and TA muscle in response to light stimulation in anesthetized ChR2 mice was successfully recorded. Remarkably, these intraspinal recording devices demonstrated strong resilience in free-moving conditions, maintaining the capability to record comparable spinal cord neuronal activities in eight months post-implantation. This long-term stability underscores the potential of hydrogel bioelectronics for extended neurophysiological monitoring in naturally behaving animals.

To address the limitations of existing hydrogel-based light delivery systems, a hydrogel fiber-optic device was developed consisting of three main components: (i) a proximal silica fiber segment encased in a ceramic ferrule and exposed to air to prevent hydrogel dehydration, with a thin poly(vinyl alcohol) (PVA) hydrogel coating on the silica to improve bonding at the silica-hydrogel interface; (ii) a hydrogel fiber composed of a PVA hydrogel core and cladding, seamlessly coupled to the silica fiber segment. To optimize light transmission along the hydrogel core, nanocrystalline domains of PVA were incorporated within the hydrogel matrix, enhancing optical clarity and reducing light scattering. Additionally, these nanocrystalline domains were anchored to the silica surface, forming a robust and fatigue-resistant interface that minimizes light loss at the silica-hydrogel junction.

The hydrogel core was fabricated by chemically cross-linking PVA within a mold with an inner diameter of 800 μm, yielding an amorphous PVA hydrogel fiber that was directly bonded to the silica fiber segment during synthesis. This configuration established a stable and compliant interface between the hydrogel fiber-optic device and the target tissue. Following fabrication, the device was acidified using a 12 mM HCl solution, air-dried, and annealed at 100° C. This annealing process converted the amorphous PVA chains into nanocrystalline domains both at the silica-hydrogel interface and throughout the bulk hydrogel, thereby enhancing the mechanical and optical properties. An annealing duration of 20 minutes was selected based on achieving an optimal balance between mechanical robustness and optical properties, such as absorbance and refractive index (RI).

### Example 3

### Hydrogels for Components Delivery.

Engineering controllable micro canals in hydrogels via ice templating. Ice templating has been successfully applied to fabricate complex composites with nacre-like morphologies, including a variety of materials such as ceramics, carbon nanotubes, graphene, and polymers. Leveraging the porous nature of ice-templated structures, directional freezing has been utilized to develop advanced materials like aerogels²²⁷, smart sponges^{228, 229}, polymeric woods²³⁰, and multifunctional cellular plastics²³¹. Recent studies have demonstrated the fabrication of robust, tough hydrogels^{232, 233} and fatigue-resistant hydrogels²³⁴ via physical cross-linking (freeze-thawing) and ice-templating techniques, enabling the formation of micro-scale structures previously challenging to achieve.

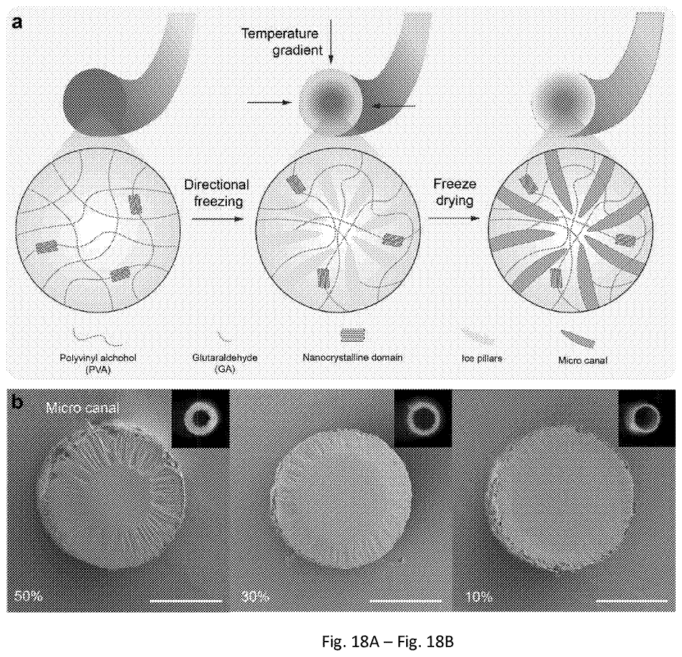

Inspired by natural icing processes and freeze-casting technologies²³⁵⁻²³⁸, freeze-derived hydrogel fibers are introduced with heterogeneous, micro-scale eccentric canals and dendritic structures (FIG. 18A). In nature, ice crystals exhibit dendritic growth patterns at the micro-scale, and the eccentric formation of these crystals can produce structured, patterned morphologies. By replicating these processes, PVA hydrogels featuring nanocrystalline domains and eccentric micro-canals were fabricated, generated through controlled directional freezing and subsequent freeze-drying.

The incorporation of a micro-scale canal structure within these hydrogels is particularly advantageous for applications in neuroscience. This unique canal morphology facilitates the transport of small molecules, ions, and drugs, making it suitable for targeted chemical delivery within neural tissues. The enhanced porosity and directional channels of the hydrogel fibers enable sustained and localized delivery of neuroactive compounds, which could aid in modulating neural activity at precise sites.

Sustained and controlled release through micro canals. Through precise control of water content within the hydrogel matrix, the length and diameter of micro-canals within the hydrogel fiber were successfully tailored, while maintaining an intact core to facilitate efficient light delivery. the PVA hydrogel fibers exhibit a high RI of approximately 1.40 when hydrated, which is crucial for achieving TIR within the hydrogel core, even as the fiber is loaded with ions, drugs, or other bioactive molecules and subsequently implanted in biological tissues. Upon implantation, fluid exchange within the micro-canal allows body fluids (RI~1.34) to occupy the patterned micro-canals, thereby creating an RI contrast at the core-canal interface that supports TIR for sustained light transmission.

The dimensions of the micro-canals, including length and width, can be precisely tuned to optimize the loading capacity for specific molecules, depending on their molecular weight and size. This structural adaptability enables the hydrogel fibers to function as a controlled-release platform for targeted chemical delivery. According to diffusion theory²³⁹, the controlled release of chemicals from the micro-canals is determined by canal dimensions, providing fine control over the release of kinetics.

In neuroscience applications, this canal-based loading and delivery system offers two major advantages. First, micro-canals allow for localized, sustained release of neuroactive compounds or therapeutic agents directly at the neural interface, enhancing the ability to modulate neural activity with spatial and temporal precision. Second, the light-guiding capabilities of the PVA hydrogel core, facilitated by TIR at the core-canal boundary, enable concurrent optical stimulation, making these hydrogel fibers suitable for multifunctional neural interfaces. By combining chemical delivery with optical functionality, this system supports advanced neuroengineering applications, including optogenetics and neural modulation therapies, where precise control over both light and chemical cues is essential for effective intervention.

Incorporation of microfluidic channels via molding and extrusion. In contrast to the sustained release mechanism offered by micro-canals, a microfluidic channel was incorporated into the hydrogel-based neural probes to enable continuous delivery of bioactive compounds for neuromodulation applications. Conventional microfluidic channel fabrication methods are limited by the narrow range of materials that can be thermally pulled due to the stringent requirements for matching glass transition temperatures (Tg) across all components⁵⁴. However, the molding and extrusion techniques, previously demonstrated for the fabrication of step-index PVA hydrogel optical fibers with thin cladding layers (FIGS. 13A-13D), provide a flexible and effective alternative.

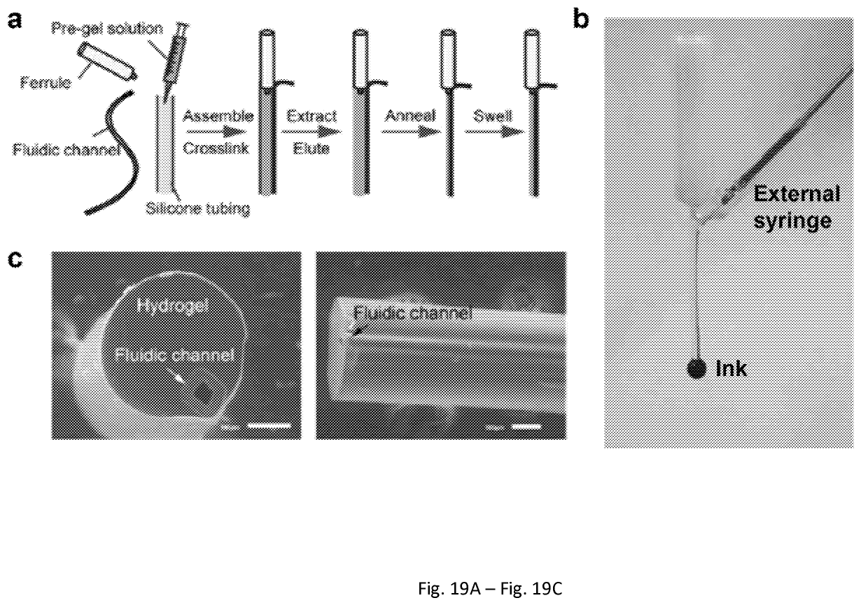

Using an 800 μm silicone mold and a 50 μm diameter microfluidic channel, the hydrogel optical core was integrated with a microfluidic delivery channel. Following insertion into the mold, an additional layer of PVA hydrogel solution was infused around the optical core and channel assembly. Chemical cross-linking of the PVA hydrogel resulted in a unified device that combines an optical waveguide and a microfluidic channel (FIGS. 19A-19C). This configuration supports dual functionalities: light transmission for optical stimulation and precise, continuous delivery of therapeutic agents.

To validate the functionality of the microfluidic channel, it was connected to an external syringe and successfully demonstrated continuous ink delivery through the channel (FIG. 19B), highlighting its capability for steady, controlled flow. This integrated device enables simultaneous optical stimulation and controlled chemical delivery, advancing the potential of hydrogel-based neural probes for applications in real-time neuromodulation and therapeutic intervention within the nervous system.

Fabrication of hydrogel optical fibers. A vacuum planetary mixer (Musashi ARV-310, 2000 rpm, and 16 kPa vacuum) was utilized for the mixing and degassing of all solutions. For degassing and mixing, 100 μL of GA was added to 10 g of 10 wt. % PVA pre-solution and agitated for 1 min. 100 μL of HCl was added to 10 g of 10 wt. % PVA pre-solution and agitated for 1 min. Subsequently, the above two solutions were combined (weight ratio of 1:1) and mixed for another minute. The resulting PVA-GA-HCl solution was infused into silicone tubes (McMaster-Carr 5236k204, 80 mm in length). After curing at room temperature for 4 h, the PVA hydrogel fibers were demolded using dichloromethane (DCM, Sigma-Aldrich, 270997, 99.8%) and incubated in HCl solutions (1-20 mM) for 2 hours at RT followed by air dried and annealing at 100° C. for 15 minutes. Finally, the hydrogel fibers were rehydrated with MilliQ water before use. This reswelling process occurred for 12 hours to achieve an equilibrium state.

Ice templating in hydrogel fibers. The fabricated fibers were contained within customized silicone molds (5 mL). Prior to freezing, the fibers were pre-cooled at 4° C. for 2 hours and subsequently subjected to freezing at −20° C. for 4 hours. Following freezing, both the fibers and any formed ice crystals were transferred to a freeze-dryer (model, −50° C., 0.021 mbar for 20 hours), ensuring the creation of porous structures within the fibers.

Integrated microfluidic channels with PVA hydrogel fibers. The PVA optical hydrogel fibers were fabricated and the PVA pre-solution was prepared using the same recipe and method. To fabricate the hydrogel device with an integrated microfluidic channel, A ferrule was attached to the proximal end of the device to provide structural support and facilitate connections for downstream applications. A silicone tubing with an embedded fluidic channel was positioned within the assembly, serving as the mold for the microfluidic pathway. The PVA pre-gel solution was then injected around the silicone tubing and ferrule assembly, followed by chemical cross-linking to solidify the hydrogel structure and lock in the fluidic channel. After cross-linking, the device was carefully extracted from the mold, and residual solvents or unreacted components were removed through an elution step, ensuring biocompatibility and mechanical stability. The device was then annealed to strengthen the hydrogel matrix, enhancing its structural integrity for extended use. Finally, the device was allowed to swell in MilliQ water for further use.

Water content fraction measurement. The water content fraction (WC %) in PVA hydrogel fibers with different HCl solutions (0, 1, 5, 10, 20 mM) were calculated based on weight. Fibers were weight after dried and reswelled, the water fraction was calculated using this formula:

$$WC \% = \frac{{W}_{r} - {W}_{d}}{{W}_{d}} \times 100 \% ,$$

where Wᵣ is the weight after reswekkedm abd W_{d} us tge weight after dried.

Porous layer percentage measurement. The porous layer percentage was measured from SEM images using image J. Each group has 4 individual samples. The formula used to calculate porous layer percentage is as follows:

$$Porous layer \% = \frac{Porous layer area}{Cross section area} \times 100 \% .$$

Virus package. AAV9-CAG-Cre was prepared in Rao Lab at UMass Amherst with Beckman Coulter Ultracentrifuge Optima XL70 with VTi 50.1 rotor. Before use, the viral vector was diluted to a titer of 10¹² transducing units per milliliter.

Implantation of AAV loaded micro patterned PVA hydrogel fibers. Mice were anesthetized using 1.0% isoflurane administered in a chamber and subsequently secured onto a stereotactic frame (RWD Life Science) with a heating pad to maintain their body temperature. All surgical procedures were conducted in sterile conditions with 1% isoflurane used to maintain anesthesia. The Allen Brain Atlas was used to align the skull and determine the coordinates for viral injection and fiber implantation, specifically targeting the ventral tegmental area (VTA) at coordinates AP: −2.95 mm, ML: ±0.50 mm, DV: −4.80 mm. An opening was made in the skull using a micro drill (RWD Life Science) at the designated coordinates. A total of 500 nL of AAV carrying AAV9-CAG-Cre was uptaken by the micro-patterned PVA hydrogel fibers at 4° C. for 2 hours. The AAV-loaded fibers were positioned on the stereotaxic table and inserted into the targeted area. Following fiber probe insertion, the probes were lifted by 0.1 mm to accommodate the viral volume. Finally, the fiber probes were secured to the skull using an adhesive (Parkell, C&B METABOND) and reinforced using dental cement (Jet Set-4). The mice were monitored on the heating pad following removal of isoflurane until they were fully awake. C57BL/6J mice (n=2) were implanted with hydrogel optical probe.

Controllable micro canal structures in PVA hydrogel fibers. To precisely control the percentage of the micro-canal layer within the cross-sections of hydrogel fibers, ice crystal growth was hypothesized to be directly correlated with the water content fraction in the hydrogel matrix. By acidifying the hydrogel matrix, intermolecular chain interactions were enhanced via hydrogen bonding²⁴⁰, allowing for fine-tuning of the water content. Following acid treatment, the hydrogel fibers were frozen at −20° C. for 4 hours, then subjected to freeze-drying for 20 hours to form micro-canals.

Upon analyzing the cross-sections of these treated fibers, the percentage of the micro-canal layer was observed to decrease proportionally with the reduction in water content. Further analysis revealed a linear relationship between water content and micro-canal layer percentage, with a slope of 0.086 and an R² value of 0.9791. This high correlation suggests that water content is directly proportional to the extent of micro-canal formation, implying that ice crystal growth can be effectively controlled through water content regulation. This approach provides a reproducible method to engineer the micro-canal layer, enhancing the structural and functional customization of hydrogel-based devices for biomedical applications, such as controlled chemical delivery and optical transmission in neural interfacing.

In vivo AAV delivery via micro canals. To evaluate the delivery capabilities of micropatterned PVA hydrogel fibers, fibers with a 50% micro-canal configuration were selected. Using fluorescent imaging, 1 μL of calcein dye was loaded into the porous canal layer and inserted the loaded fiber into a brain phantom containing 0.6% agarose gel. Under blue light excitation, the/U temporal release of the dye was visualized from the micro-canal structure. Analysis of the fluorescent diffusion area revealed that the dye diffused effectively into the agarose matrix, reaching a stable release point after approximately 10 minutes. This result demonstrates the micro-canals' efficacy in controlled release, supporting their potential for localized delivery in biological environments.

To further assess the in vivo delivery capability of micro-canal fibers, 500 μL of AAV9-CAG-Cre viral vector was loaded into the 50% canal layer and implanted the fiber into the VTA of Ai14 mice. Ai14 mice were specifically chosen for this experiment due to their genetic expression of the tdTomato reporter in Cre-recombinase-expressing cells, allowing direct visualization of successful AAV-mediated gene delivery²⁴¹. Five days post-implantation, the VTA region was analyzed for tdTomato expression using immunofluorescence. Robust tdTomato expression was observed in numerous neurons within the VTA, indicating efficient viral delivery through the micro-patterned hydrogel fiber.

These findings highlight the potential of micro-canal fibers for both controlled, time-dependent release in vitro and effective, localized viral vector delivery in vivo. The Ai14 mouse model provides a sensitive platform for confirming AAV delivery, as successful Cre-mediated recombination in the VTA results in clear tdTomato expression, underscoring the capability of the micro-patterned fibers to serve as advanced neuromodulatory and gene delivery tools in neural research and eventually clinical application.

Micro-patterned PVA hydrogel fibers featuring a controlled micro-canal layer for precision delivery applications were developed and characterized. By fine-tuning the water content in the hydrogel matrix and employing freeze-drying techniques, reproducible formation of micro-canals was achieved within the fiber structure. The direct correlation between water content and micro-canal formation enables predictable customization of the micro-canal layer, enhancing the fiber's functionality for controlled release. The in vitro experiments demonstrated effective diffusion of a model dye, and in vivo application in Ai14 mice confirmed successful, localized gene delivery to the VTA, as evidenced by robust tdTomato expression. These findings validate the potential of micro-patterned hydrogel fibers as versatile tools for targeted delivery in neural and other biomedical applications. The micro-canal configuration within the PVA hydrogel fibers offers a powerful mechanism for controlled and localized delivery of therapeutic agents, viral vectors, and other bioactive molecules. The micro-canals serve as defined channels within the hydrogel matrix, which facilitate the storage and timed release of payloads when exposed to target environments. By precisely modulating the percentage of the micro-canal layer, a release profile suitable for both immediate and sustained diffusion was achieved.

The versatility of micro-canal hydrogel fibers opens new avenues for biomedical applications, particularly in fields requiring precise spatial and temporal control of bioactive agents. In regenerative medicine, these fibers could be employed to deliver growth factors or stem cells to damaged tissues, providing a controlled release mechanism that enhances healing outcomes. In oncology, micro-canal hydrogel fibers could enable targeted chemotherapy or immunotherapy, releasing drugs directly at tumor sites to maximize therapeutic efficacy while minimizing systemic toxicity. In neural engineering, these fibers allow for localized delivery of neuromodulatory drugs or genetic material to specific brain regions, permitting targeted manipulation of neural circuits and supporting advancements in brain-machine interfaces.

### Example 4

### ALD Coated Hydrogel Neural Probe

The integration of soft materials in neural probe fabrication represents a promising strategy for creating neural-material interfaces with mechanical properties that align closely with delicate neural tissues. Hydrogels, known for their high-water content, biocompatibility, and flexibility, are particularly well-suited for interfacing with neural structures²⁴². Among these, cross-linked PVA hydrogels have gained considerable attention in neural probe applications due to their tunable optical and mechanical properties, biocompatibility, and capacity to adapt to neural tissue, thereby minimizing the tissue damage and inflammation often associated with conventional neural probes⁷⁹. However, the amorphous PVA chains within semi-crystalline hydrogels tend to stretch upon water absorption, resulting in gradual structural swelling, especially at elevated temperatures. These swelling compromises the dimensional integrity and long-term functionality of hydrogel-based bioelectronic devices. Therefore, developing a strategy to enhance hydrogel stability against swelling under physiological conditions is crucial for maintaining the durability and reliability of hydrogel-based neural devices.

Various techniques have been explored to stabilize hydrogels, including chemical cross-linking, incorporation of reinforcing agents, chemical vapor deposition, and atomic layer deposition (ALD) 243. Deposition-based methods, particularly ALD, offer precise control overcoating thickness and composition, while overcoming the geometric limitations of soft materials. ALD is particularly advantageous for PVA due to its relatively low processing temperatures (below 200° C.), which prevent decomposition of PVA (315° C.). Additionally, ALD facilitates the formation of conformal, pinhole-free coatings that improve the mechanical properties and biostability of hydrogels. The high processing temperature (175° C.) of ALD promotes nanocrystalline growth, further enhancing the stability of PVA hydrogel fibers without compromising their optical properties. ALD-based SiO₂ coatings, known for their high transparency, add additional benefits by preserving light transmission.

To address the swelling issue in hydrogel optical fibers, chemically cross-linked PVA hydrogel fibers coated with SiO₂ layers using ALD to enhance their structural stability were fabricated. The SiO₂-coated PVA fibers displayed nanocrystalline domains with an average size of 6.5±0.1 nm, maintaining stable dimensions under accelerated stability tests (incubated at 37° C., pH=4, and at 45° C., pH=7 in PBS for one week). The coated fibers achieved an average elastic modulus of 73.7±31.7 MPa and a maximum elongation of 113.6±24.2%, with moduli orders of magnitude lower than those of traditional optical fibers, thus facilitating compatibility with soft tissue (cite). Additionally, the SiO₂-coated PVA hydrogels exhibited high transparency (92.7±0.5%) and low light transmission loss (1.9±0.2 dB/cm). These coated fibers showed stable mechanical and optical properties post-accelerated stability tests compared to uncoated PVA fibers, underscoring their resilience in challenging environments. The SiO₂-coated PVA hydrogel fibers were subsequently used in vivo for optogenetic experiments, where they were implanted in the motor cortex (M2) of Thy1::ChR2 mice. When optically stimulated at 470 nm, 26 mW/mm², with 10 ms pulses at 20 Hz, the SiO₂-coated fibers effectively modulated locomotor behavior. The mice exhibited optically controlled contralateral rotation, increased locomotion speed, and enhanced travel distances, demonstrating the capability of SiO₂-coated PVA fibers to support stable, functional optogenetic modulation. This work highlights the potential of ALD-coated PVA hydrogel fibers as advanced tools for optoelectronic neural interfaces with enhanced durability and functionality in vivo.

Inorganic coatings enable structural stability of hydrogel optical fibers in vivo. By applying ALD coatings, it is possible to create a thin barrier layer on hydrogel fibers that controls the permeability of gases and liquids. This can be beneficial to prevent hydrogel from swelling to compress tissues when applied in biological bodies. ALD can also improve the mechanical strength of hydrogel fibers, which are often soft and prone to damage under stress. The nanoscale coating layer helps increase durability without significantly adding to the bulk, preserving flexibility while providing reinforcement. ALD coatings can be designed to enhance optical properties, such as transparency or reflectivity, which may be useful in optical sensors. A key advantage of deposition-based techniques is their ability to overcome geometric and configuration limitations of soft material devices while providing precise control over thickness and composition. Utilizing the ALD technique to create conformal and pinhole-free coatings on the surface of hydrogel fibers results in improved mechanical properties and biostability of the hydrogels.

To fabricate a heterogeneous hydrogel fiber and increase the stability of hydrogel for biomedical applications, the stable nature of SiO₂ was leveraged and the ALD process (175° C., 1.5 hours) used to coat a uniform and conformal SiO₂ coating layer on PVA fibers. To confirm the success of the ALD approach, the cross-section of SiO₂-coated PVA fibers was examined using a SEM and detected the formation of silicon element at the periphery through EDS.

To demonstrate the versatile applicability of the ALD technique, chemically cross-linked polyacrylamide (PAAm, 50 wt. %) hydrogel membranes were prepared and a SiO₂ layer applied using ALD. Cross-sectional analysis of the coated PAAm hydrogel membranes via SEM, combined with EDX mapping, confirmed the presence and uniform distribution of Si, indicating successful deposition of the SiO₂ layer.

Given the nanoscale nature of the ALD coating, its durability was assessed under challenging conditions to ensure it could withstand potential wear and tear during use as optical hydrogel fibers. To this end, SiO₂-coated PVA hydrogel optical fibers were subjected to a harsh acidic environment at 45° C. for one week. SEM and EDS analyses post-treatment revealed that the ALD coating remained intact, demonstrating the robustness of the SiO₂ layer in preserving the structural integrity of the hydrogel fibers under extreme conditions.

Preparation of PVA hydrogel pre-solutions. To prepare a 10 wt. % PVA solution, 10 g of PVA powder was dissolved in 90 ml of MilliQ water under continuous stirring in a water bath at 100° C. for 4 hours. For the preparation of pre-solutions for chemical cross-linking, 110 μL of GA was added to 10 g of the PVA pre-solution and the mixture was homogenized in a mixer for 1 minute at 2,000 rpm under a vacuum level of 16 kPa to ensure uniformity. Subsequently, a solution for the reaction accelerator was prepared by adding 150 μL of HCl to 10 g of the 10 wt. % PVA pre-solution, followed by the same mixing procedure to ensure complete mixing.

Fabrications of optical hydrogel fibers. Silica fibers and optical ferrules were employed to create a junction for light coupling of optical hydrogel fibers. The silica fiber was first separated using a diamond cutter and then cut into 13 mm short fibers. These fibers were then inserted into the ferrule and carefully pulled out from one end to obtain a 2.5 mm length of silica fiber, where the hydrogel can be attached later. EccoBond F adhesive was utilized to secure the silica fibers, followed by polishing to ensure effective light transmittance. The light transmission efficiency of the silica fibers was assessed by coupling with a 470 nm blue light-emitting diode (LED). To improve the adhesion between silica fibers and PVA hydrogels, a thin layer of PVA on the extruded silica fibers was achieved by dip coating them in a 10 wt. % PVA pre-solution, followed by air drying at room temperature (RT) overnight and annealing at 100° C. for 1 hour. To fabricate chemically cross-linked PVA optical fibers, PVA-GA and PVA-HCl pre-solution were mixed (weight ratio 1:1) under the same mixing conditions. Afterwards, the PVA-GA-HCl solution was injected into silicon tubes and optical ferrules were inserted into the tubes on the opposite end. The PVA-GA-HCl fibers were allowed to cross-link at RT for 2 hours. To elute the fiber from the silicone tubes, DCM was used to swell silicon tubes. Subsequently, the fibers underwent a two-day washing process with a significant volume of water to eliminate any residual chemicals. The ferrule-connected fibers were immersed in HCl solutions (12 mM) for 2 hours followed by air drying at RT overnight and annealing at 100° C. for 20 minutes.

Preparation of Polyacrylamide (PAAm) hydrogels. 7.5 g of PAAm powder was dissolved in 7.5 mL of MilliQ water, 0.1 g of BA was dissolved in 4.9 mL of MilliQ water, 0.5 mL of TEMED was dissolved in 4.5 mL of MilliQ water, 1.5 g of APS was dissolved in 13.5 mL of MilliQ water. To prepare chemically cross-linked PAAm hydrogels, PAAm, 1×PBS, BA, TEMED, and APS were mixed together with a volume ratio of 1:1:0.01:0.03:0.03. The mixed solution was poured in a rectangle mold (1× 1× 1 mm) and allowed to cross-link at RT for 1 hour.

Atomic layer deposition to maintain the structural stability of hydrogel optical fibers. As fabricated PVA hydrogel fibers were washed with large amount of water to remove excess HCl and air dried at RT for 12 hours. To deposit a single layer of SiO₂ onto dried PVA hydrogel fibers, an ALD instrument (Anric Technologies AT-400) was employed. The dried PVA hydrogel fibers were mounted on a silicon substrate and placed inside the PECVD chamber. The chamber was then pumped down to a base pressure of 10-6 Torr. Tri (dimethylamino) silane gas was introduced into the chamber as the precursor, and the deposition process was carried out for 100 cycles (one and a half hours) at 175° C.

Ellipsometry. Hydrogel membranes were produced using a spin coating method (KW-4A Spin, SETCAS) on square silicon (Si) substrates (15 mm×15 mm) that were cleaned by washing and ultrasonication in acetone, 30 wt. % H₂SO₄ solution, and 10 wt. % H₂O₂ solution, followed by rinsing with MilliQ water and 95% ethanol to ensure hydrophilic surfaces. PVA-GA-HCl pre-solution were prepared following the same procedure above and membranes were prepared on the Si substrates using the spin coater at 1000 rpm for 10 s, followed by 5000 rpm for 50 s. The resulting membranes were cross-linked and dried for at least 12 hours, followed by annealing at 100° C. for 20 minutes. The refractive index of the PVA membrane-coated Si wafers was then measured using an ellipsometer (J.A. Woollam RC2) in the range of 400 nm to 600 nm after aligning and calibrating the reflection beam for each wafer before measuring. To determine the refractive index (RI) and thickness of the PVA membranes, a Cauchy film fitting model was utilized. Additionally, the transmittance of PVA membranes was calculated using the following formula:

$${T}_{max} = \frac{2 n}{{n}^{2} + 1} ,$$

where n is the RI and Tₘₐₓ represents the maximum transmittance of PVA thin films.

Characterization of contact angles on hydrogel thin films. The contact angle of hydrogel thin films was characterized using the sessile drop method. Briefly, hydrogel thin films were prepared on silicon substrates using a spin coating method described above. The substrates were then mounted on a goniometer stage, 3 μL of MilliQ water was carefully dispensed onto the surface of the hydrogel thin film. A camera was used to capture images and the contact angle was determined by measuring the angle between the tangent of the droplet interface and the surface of the hydrogel film. Five independent measurements were taken for each hydrogel thin film, and the results were averaged to obtain the contact angle value.

Light transmission tests of optical hydrogel fibers. The optical transmission spectra were tested by implementing a cutback method, which ensures that coupling efficiency of the light source remains precise throughout all cutback measurements. The hydrogel fiber was followingly placed into a plastic tube with dimensions of 5 cm in length and 3 mm in diameter. 1 wt. % agarose gel was then injected into the plastic tube to prevent loss of hydration of the hydrogel optical fibers. A 470 nm LED light was then connected to the ferrule attached to the end of the hydrogel fiber using an adaptor. To obtain measurements for the intensity of light transmittance at different fiber cutbacks, a power meter was used. The cutback measurements were taken on an interval of 0.5 cm with the uncut fiber length as the initial output power measurement. After gathering output power measurements at each 0.5 fiber cut, the coefficient of attenuation (a, dB/cm) was calculated using the following formula:

$$α = ( \frac{1 {0}^{4}}{{L}_{1} - {L}_{2}} ) \cdot log ( \frac{{P}_{1}}{{P}_{2}} ) .$$

In this formula, L₁ represents the original length of the fiber before the respective cut is done, L₂ represents the length of the fiber after the cut, P₁ represents the transmitted light power before the cut, and P₂ represents the transmitted light power after the respective cut on the fiber.

Characterization of mechanical properties. Before conducting the extension test, all fibers were kept hydrated. A Stable Micro System (TA. XT plusC, 50N load cell) was employed to stretch the hydrogel fibers using tensile tests at a rate of 1 mm/second. The nominal stress was calculated using the formula σ=F/A, where F denotes the recorded force and A denotes the cross-sectional area of the fibers in the hydrated state. The strain was calculated using: ϵ=ΔL/L, where ΔL represents the displacement, and L represents the gauge length. The elastic moduli (E) were determined by calculating the average slope of the stress-strain relationship in the first 10% of applied strain using linear regression. The stretchability (%) of the fibers was reported at the point of fracture in the stress-strain curve.

Biocompatibility test of optical hydrogel fibers. The HEK 293 FT cell line was used for biocompatibility test and cultured in DMEM supplemented with GlutaMax and 10% FBS and seeded in a 24-well plate. Optical hydrogel fibers (pre- and post-Si coating) were incubated in DMEM for 24 hours at 37° C. After incubation, the hydrogel-incubated DMEM was added to the well plate and incubated for an additional 24 hours. Living cells were labeled with Calcein-AM (green), and dead cells were labeled with Ethidium homodimer-1 (red). Images of the cells were captured using a fluorescent microscope (Nikon TiU) and image J was used for cell counting statistics analysis.

In vivo implantation of optical hydrogel fibers. All animal surgeries were reviewed and approved by the Committee on Animal Care at the University of Massachusetts Amherst. Thy1::ChR2-EYFP mice were procured from the Jackson Laboratory and were provided ad libitum access to food and water. The mice were housed in a controlled environment at 24° C.±1° C., with 50% relative humidity, and on a 12-h light/12-h dark cycle, with all experiments conducted during the light cycle. Thy1::ChR2-EYFP mice were anesthetized with 1.0% isoflurane and secured onto a stereotactic frame (RWD Life Science) with a heating pad to maintain body temperature. Surgical procedures were performed under sterile conditions with continuous 1% isoflurane anesthesia. The Allen Brain Atlas was used to determine the coordinates for fiber implantation in the M2 cortex region (AP: 2.50 mm, ML: ±1.00 mm, DV: −1.00 mm). After drilling a hole in the skull at the designated coordinates, hydrogel fibers were implanted while the silica and ferrule were secured using an adhesive and reinforced using dental cement (Parkell, C&B METABOND and Jet Set-4). Following fiber insertion, the mice were monitored on the heating pad until they regained full consciousness.

Optogenetic stimulations and behavioral assays. A blue laser light (473 nm) was employed for illumination and light was delivered into the mice through a ferrule-sleeve-ferrule connecting system. Light stimulation protocols were generated using a function generator (xx mW/mm², 10 ms pulse and 20 Hz). Prior to the experiment, mice were habituated to the testing chamber for at least 30 minutes. Optogenetic sessions for experiments were conducted with a 9-minute period divided into three 3-minute epochs, with the first epoch serving as a baseline for locomotor activity, while the first and third epochs were designated as light-OFF periods. The second epoch served as the light-ON period, allowing for the recording of the effects of optogenetic modulation. A camera was utilized to record mouse behaviors during optogenetic stimulation. ToxTrac^{18,21} was used to analyze speed change, travel distances and trajectories of mice during each epoch. Rotation behaviors were classified as ipsilateral (same side of the implantation site) or contralateral (opposite side of implantation site) rotations around the body axis. The videos were manually scored independently by another researcher and the frequency of rotations was assessed for each epoch.

Immunohistology. Transcardiac perfusion was performed on euthanized mice using a PBS solution followed by a 4% paraformaldehyde (PFA) solution. The brains were fixed in 4% PFA solution overnight at 4° C., and subsequently treated with 30% sucrose in PBS for 2 days before being frozen in an O.C.T. cube and sectioned (20 μm) using a cryostat (Leica CM1900). The sectioned tissues were permeabilized in PBST (0.3% Triton-X-100 in PBS) and blocked with bovine serum albumin (1 wt. % BSA in PBS) prior to staining with primary antibodies (Ibal Rabbit and GFAP Rabbit) overnight at room temperature. Secondary antibody solutions were then applied and incubated at room temperature for 2 hours, followed by washing with PBS and mounting on glass slides using DAPI mounting medium. The samples were left to dry overnight at room temperature before being imaged using a confocal microscope. The primary antibodies were used at a dilution of 1:400 in PBS, while the secondary antibodies were used at a dilution of 1:200 in PBS. The GFAP secondary antibody used was Donkey anti-Rabbit IgG (H+L) Highly Cross-Absorbed Secondary Antibody Alexa Fluor 488 Invitrogen (green), while the Ibal secondary antibody used was Donkey anti-Rabbit IgG (H+L) Highly Cross-Absorbed Secondary Antibody Alexa Fluor 555 (red).

Statistical Analysis. Statistical analyses were performed in Excel (Microsoft Corporation, Inc.), Prism (Graphpad Software, Inc.), and Toxtrac.

Fabrication of heterogeneous PVA hydrogel fibers. To utilize the chemical cross-linking of PVA hydrogels, GA was used as the chemical cross-linker and combined with the molding-extrusion method to fabricate PVA hydrogel fibers¹¹⁵. To fabricate a heterogeneous hydrogel fiber and increase the stability of hydrogel for biomedical applications, the stable nature of SiO₂ was leveraged and the ALD process (175° C., 1.5 hours) used to coat a uniform and conformal SiO₂ coating layer on PVA fibers. Since the degree of crystallinity in PVA nanocrystalline materials is vital for stability, wide-angle X-ray scattering (WAXS) was conducted to analyze the nanocrystalline size of the coated PVA. The SiO₂-coated PVA fibers exhibited a higher degree of crystallinity compared to both uncoated PVA fibers (annealed at 175° C. for 1.5 hours) and pure PVA (unannealed) fibers. Since accelerated stability testing is used under harsh environment which is more severe than normal physiological conditions,^{23,24} SiO₂-coated PVA fibers and uncoated PVA fibers were subjected to accelerated stability tests (incubations under 37° C. pH=4 and 45° C. pH=7 in PBS for one week) to assess the stability of SiO₂-coated PVA fibers. Following incubations, SiO₂-coated PVA fibers maintained consistent fiber diameters with less than a 20% diameter change, while uncoated PVA fibers exhibited significant diameter changes (40.4±16.6%) after incubation under 45° C. pH=7. This result can be attributed to the lower crystallinity in uncoated PVA fibers and the SiO₂ coating serving as a barrier against environmental changes. Lastly, contact angle tests were conducted with water on SiO₂-coated and uncoated PVA hydrogel thin films to determine if the SiO₂ coating affected the swelling behavior and hydrophilicity of PVA hydrogels. The contact angle measurements remained consistent between groups, indicating that the SiO₂ coating did not negatively impact the hydrophilicity of PVA hydrogels.

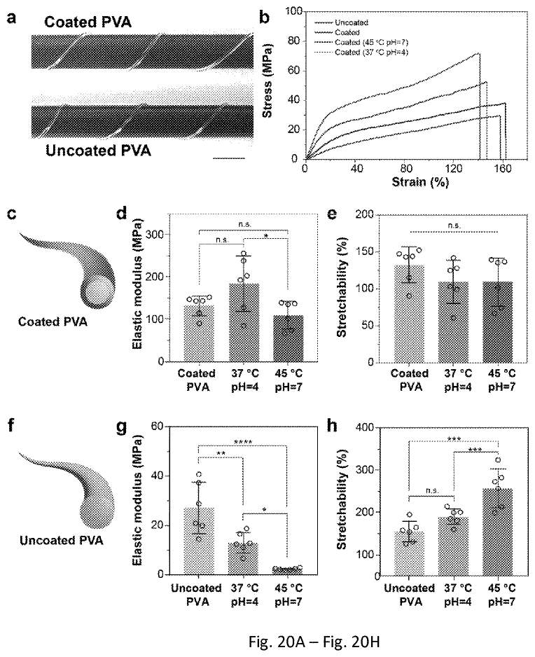

Mechanical performance. Having confirmed the dimensional stability of SiO₂-coated PVA hydrogel fibers under various conditions, the impact of the ALD process on the hyper-elastic properties of PVA hydrogel fibers (FIG. 20A) was investigated. Tensile tests were conducted on two sample groups: SiO₂-coated and uncoated PVA hydrogel fibers (FIG. 20B). The coated fibers demonstrated increased fracture stress and improved elastic modulus compared to uncoated fibers (FIGS. 20B, 20D, and 20G). This enhancement can be attributed to the facilitation of nanocrystalline growth during the ALD process and the presence of the SiO₂ layer. However, the elastic modulus of the coated fibers is three orders of magnitude lower than that of traditional polymer-based optical fibers⁶⁴, providing improved flexibility and reduced tissue damage for in vivo applications. After verifying the dimensional stability of SiO₂-coated fibers following accelerated tests (incubations under 37° C. pH=4 and 45° C. pH=7 in physiological solutions for one week), tensile tests were conducted on incubated SiO₂-coated fibers to examine the stability of their mechanical properties. The coated groups displayed relatively consistent elastic moduli (FIG. 20D) and stretchability (FIG. 20E), while the uncoated groups exhibited a decreased elastic modulus (FIG. 20G) and increased stretchability (FIG. 20H). These findings indicate that the SiO₂ coating enhances the stability of PVA hydrogel fibers.

Optical properties. To ensure optimal light transmission for optical stimulation, two key parameters of the PVA hydrogel materials were systematically assessed: light transmittance and light attenuation. Initially, PVA hydrogel thin membranes were fabricated utilizing the spin coating technique, followed by the application of a SiO₂ coating (175° C., 1.5 hours). The thickness of the SiO₂ coating was precisely examined using ellipsometry, which was 14.56±4.69 nm. To investigate the controllable thickness of SiO₂ through the ALD process, Si wafers were subjected to the ALD process with different processing time. The thickness of SiO₂, measured by ellipsometry, indicates a linear relationship between the SiO₂ coating thickness and the ALD cycle numbers. The refractive indices of both the dried PVA hydrogel membrane and the SiO₂ coating were characterized using ellipsometry. The refractive index was found to be 1.44±0.04 for the PVA hydrogel membrane and 1.50±0.01 for the SiO₂ coating. The analysis demonstrated no significant differences in transmittance at wavelengths of 470 nm and 510 nm between the uncoated and SiO₂-coated PVA hydrogel membranes, indicating compatibility with optogenetic stimulation requirements in different ranges. Subsequent characterization of the light transmission properties of the optical fibers revealed that SiO₂-coated PVA hydrogel fibers possessed an expanded numerical aperture in comparison to uncoated PVA fibers, as demonstrated by light transmission tests conducted from a SiO₂-coated PVA fiber into a brain phantom (0.6% agarose) containing calcein fluorescent dye. Since optogenetics stimulation requires a low light loss and considering the application for in vivo optogenetics stimulation targeting ChR2, a blue LED at a wavelength of 470 nm was used. The cut-back method was used to systematically characterize the light transmission in PVA fibers by normalizing transmission (from the tip of PVA fibers after each cut) as a function of fiber length for PVA fibers. The light attenuation coefficient of the SiO₂-coated PVA hydrogel fibers (307.5±20.3 μm, 1.91±0.16 dB/cm) exhibited no significant decrease compared to annealed PVA fibers (328.6±17.7 μm, 1.29±0.20 dB/cm). To investigate the stability of optical properties of SiO₂-coated PVA fibers, the light attenuation of SiO₂-coated PVA hydrogel fibers post accelerated stability tests was characterized. In contrast to SiO₂-coated PVA fibers prior to the incubation, the group under 37° C. pH=4 incubation (283.1±9.7 μm) and the group under 45° C. pH=7 incubation (296.8±16.5 μm) exhibited a light attenuation coefficient of 1.69±0.38 dB/cm and 1.52±0.23 dB/cm, respectively, displaying no significant difference. This provides the potential for later in vivo optogenetic applications.

Cytotoxicity assessment. Prior to implanting SiO₂-coated PVA hydrogel fibers for in vivo applications, a comprehensive evaluation of their biocompatibility through cytotoxicity evaluation was conducted. Cytotoxicity testing is a crucial step in determining the safety and suitability of a material for biological applications, particularly when considering implantation in living organisms²³². For cytotoxicity testing, HEK 392T cells were used, a commonly employed cell line for biocompatibility evaluations due to their well-characterized behavior and easy maintenance (cite). The cytotoxicity of SiO₂-coated and uncoated PVA fibers was compared by examining cell viability after indirect contact with the materials (cells were cultured by PVA hydrogel fiber incubated media). Following incubation with both SiO₂-coated and uncoated PVA fibers, cell viability was assessed under the optical microscope. Calcein-AM (green) was used to stain living cells and ethidium homodimer-1 (red) was used to stain dead cells, the observations revealed no significant differences in cell death between SiO₂-coated PVA fibers and uncoated PVA fibers (FIG. 6-5j). This result suggests that SiO₂-coated PVA fibers exhibit good biocompatibility and are likely safe for use in in vivo applications.

Optogenetic activation enabled by PVA hydrogel fibers. To investigate the functionality of PVA hydrogel fibers for in vivo optogenetic stimulation, SiO₂-coated fibers and uncoated fibers in the M2 cortex region (FIG. 4c-d) of Thy1::ChR2-EYFP mice were implanted and motor control behavioral assays performed. The M2 cortex is a critical region for optogenetic stimulation and motor control studies due to its significant role in the initiation, planning, and execution of voluntary movements^{175, 244}. SiO₂-coated fibers and uncoated fibers were unilaterally implanted in Thy1::ChR2-EYFP mice and three days after implantation, monitored the mice for changes in locomotor activity in response to optogenetic excitation of M2 neurons in a customized chamber. Blue light (473 nm, 26 mW/mm², 10 ms pulse, and 20 Hz) was delivered through the optical ferrule into the M2 cortex with three 3-min stimulation epochs: the first session was the pre-stimulation (pre-stim) light OFF, the second was during-stimulation (dur-stim) Light-ON, and the third was post-stimulation (post-stim) light off. To evaluate whether the coated fiber can reliably stimulate M2 cortex and its excitatory projections in locomotor behaviors, the mice behaviors during each epoch were recorded. Compared with Light-OFF sessions, the optical activation of ChR2 reliably evoked an increase in contralateral rotations around the test chamber during the Light-ON periods in both test and control animals, with this motor-evoked response being immediate after light onset. Rotation behaviors were classified as ipsilateral (same side of the implantation site) or contralateral (opposite side of implantation site) rotations around the body axis. The frequency of rotations per 3-min epoch was assessed and the statistical results indicated a significant increase in contralateral rotations (F_{(2,36)}=29.9, ****p<0.0001) and a non-significant change in ipsilateral rotations (F_{(2,36)}=1, n.s. p=0.3779). On average, speed during the light-ON periods was significantly greater than in light-OFF epochs (FIG. 5d, F_{(2,21)}=10.21, ***p=0.0008). The test mice also exhibited an increase in total distance traveled during light stimulation (FIG. 5e, F_{(2,21)}=10.12, ***p=0.0002). In a separate group of animals, control mice (implanted with uncoated fibers) were subjected to the same optogenetics stimulation regime and motor control behavioral assays (FIG. 4h). The control mice presented relatively similar behavior patterns in response to the optogenetic stimulation (FIG. 5g-j, 473 nm, 28 mW/mm², 10 ms pulse width, and 20 Hz). Particularly, there were significant increases in contralateral rotations (F_{(2,48)}=7.845, **p=0.0011), average speed (F_{(2,21)}=13.02, ***p=0.0002), and total travel distance (F_{(2,21)}=10.02, ***p=0.0009), while ipsilateral rotations showed non-significant changes (F_{(2,42)}=3.5, n.s. p=0.0393). Together, these results demonstrate the viability of PVA hydrogel fibers for optogenetic stimulation and enable the optogenetic activation of M2 neuronal bodies and their axons to evoke locomotor activity.

The in vivo biocompatibility of the PVA hydrogel fibers was evaluated by performing immunochemical quantification of markers indicative of astrocytes (glial fibrillary acidic protein, GFAP) in the M2 region around the implantation sites. This assessment provided valuable insight into the host tissue response to the PVA hydrogel fibers, as the presence of activated astrocytes is a common indicator of neuroinflammation after biomaterial implantation⁷⁹. The findings revealed no significant differences in the extent of astrocyte activation (GFAP expression) between the coated and uncoated PVA hydrogel fibers as observed through confocal microscopy. The absence of significant differences between the coated and uncoated fibers suggests that the base PVA hydrogel material exhibits inherent biocompatibility, which is not affected by the applied coating. This characteristic is advantageous for various neural tissue engineering applications, as it minimizes the likelihood of negative host tissue reactions upon implantation.

In conclusion, SiO₂-coated PVA hydrogel fibers were successfully fabricated using GA as the chemical cross-linker and the molding-extrusion method. The ALD process provided a uniform and conformal SiO₂ coating layer, which significantly enhanced the stability and mechanical properties of the fibers. The coated fibers displayed improved crystallinity and dimensional stability under various conditions, while maintaining hydrophilicity and light transmission properties. Importantly, the SiO₂-coated PVA fibers exhibited good biocompatibility in cytotoxicity assays, suggesting their safety for in vivo applications. Through in vivo optogenetic stimulation experiments, the effectiveness of these fibers in modulating motor control behavior in Thy1::ChR2-EYFP mice was demonstrated, indicating their potential application in neural circuits and their implications in various neurological disorders. The development of SiO₂-coated PVA hydrogel fibers presents an advancement for the stability of hydrogel-based optical fibers in the field of optogenetics, offering a flexible, biocompatible, and efficient tool for the exploration of the brain's neural circuits and the potential for neuromodulation in other regions such as the peripheral nerve system.

### Example 5

### COMPACT Hydrogel Neural Probes

Integration and Miniaturization of Hydrogel Neural Probes. Soft materials bioelectronics enable the interrogation of biological function from single-cell to organ-level resolution¹. In dynamically moving in vivo environments, soft bio-interfaces can adapt to the persistent mechanical deformations of the living tissues, and consequently provide reliable access to biological systems. For the sophisticated yet delicate nervous system interfaces, elastic polymer materials, including polydimethylsiloxane (PDMS), cyclic olefin copolymer elastomer (COCE), polyurethane (PU), and hydrogels have been deployed as the suitably elastic substrate for multifunctional devices that enable neural optogenetics stimulation, electrophysiological recording, drug infusion and neurotransmitter detection. However, fabricating dedicated microstructures in soft and elastic devices is limited to two-dimensional (2D) architectures and heavily relies on sophisticated manufacturing approaches such as lithography and micro-printing.

The thermal pulling method yields multiple-step scaling-down feasibility for multifunctional polymer fibers, and this approach requires coherent parameters of the constituent materials, such as glass transition temperature, melting temperature and thermal expansion coefficients to be drawn into an integrated fiber. Moreover, the high-temperature process narrows the selections of available polymers for high-water-content bioelectronics. Assisted with hydrogel cross-linking as a soft material matrix, hybrid multifunction fibers permit adaptive bending stiffness for long-term sensing and neural modulation.

Besides mechanical stiffness changes in the hydrated state and the desiccated state, hydrogel materials permit tunable volumetric control as the supporting scaffold. Hydrogel swelling behaviors in response to external stimuli have enabled drug release, ingestible devices, and expansion microscopy to enhance microimaging resolution. Anti-swelling hydrogels are usually constructed by regulating the cross-linking, hydrophilicity/hydrophobicity balance and nanocomposite for tissue engineering applications. Meanwhile, hydrogel shrinking behaviors in a desiccated state have been applied to densify patterned materials in volumetric scaffold deposition and obtain nanoscale feature sizes in three dimensions. However, the hydrogel swelling and shrinking behaviors in these techniques are based on reversible polymer chains collapse in the desiccated state and expansion upon hydration. When applied to an aqueous in vivo environment, the shrunk hydrogels will expand and lose the miniaturized structures from the original manufacturing.

Inspired by the volumetric change resulting from polymer chains' folding and expansion, a hypothesis was tested centered on controlling the amorphous-crystalline transition in semi-crystalline hydrogels. By intervening in the polymer chain folding and crystallization process, the expansion of polymer chains from their nanocrystalline structure is limited, and consequently hydrogels enabled to preserve their designed volumes under a solvated state. Utilizing the nanoscale structure change to regulate soft materials properties has been proven as an effective approach to biomedical applications. PVA hydrogels, one of typical semi-crystalline polymers, have been extensively used in drug release, food packaging and wound healing owing to their high-water content, transparency, and biocompatibility. In semi-crystalline polymer matrices, the swelling behavior involves water molecule diffusion, amorphous polymeric chain relaxation via hydration, and expansion of the cross-linked polymer network. To finely tune polymeric crystallization processes, engineering approaches that impact polymer chain interactions, solvent evaporation, and external stretching can be employed to facilitate molecular chain arrangement³⁵. Moreover, PVA polymer matrix can be incorporated with nanomaterials to enhance mechanical strength, conductivity, and biocompatibility. For PVA hydrogel bioelectronics, controlling nanostructures through polymeric crystallization approaches can enhance the stability to maintain their designed architecture in biological environments.

A set of cross-linking chemistry and micro-fabrication processes were developed to control polymeric crystalline domain growth with cross-linked PVA hydrogels. A stable and tunable volumetric decrease of hydrogels was consistently achieved in a hydrated state under physiological conditions (pH 6-8, 37° C.). Through acidification treatment that affects polymer chain interactions while introducing dual cross-linkers of the inorganic binder TEOS and the generic GA, the polymetric crystalline scattering (crystal size around 3.5 nm) was minimized and the hydrogels' refractive indices (RI) increased. Further nanocrystalline orientation induced by uniaxial deformation promoted the generation of nanoscale anisotropic architectures. This control of metamorphic polymers' amorphous-crystalline transition (COMPACT) strategy enabled a 79.7% diameter decrease of hydrogel fibers in the hydrated state. Taking advantage of these tunable hydrogel matrix scaffolds, conductive nanomaterials, CNTs, were loaded into COMPACT hydrogels to fabricate soft microelectrodes, and tested its functionalities for electrophysiological recordings of spontaneous neural activity in the mouse brain. Integrating a hydrogel optical core and CNTs-PVA microelectrodes, multifunctional hydrogel optoelectronic devices were developed and the simultaneous optogenetic stimulation and electrophysiological recording of optically triggered neural activities in transgenic Thy1-ChR2-EYFP mice demonstrated.

To enhance electrophysiological recording capabilities, incorporating only two electrodes within a miniaturized device proves insufficient. Additionally, due to the incorporation of hair-thin hydrogel microelectrodes in the thin cladding layer, along with the size mismatch between the optical core and hydrogel microelectrodes, peeling has become a significant issue. To address this, it was hypothesized that using a dip-coating method combined with a volumetric shrinking approach—leveraging centripetal and isotropic shrinking, could effectively coat a conductive functional layer around an optical core. This configuration would not only provide a larger surface area for electrophysiological recordings but also mitigate peeling issues by promoting polymer chain infiltration between the optical core and the conductive layer.

Spinal cord injuries are frequently associated with significant loss of organ function or voluntary limb control. Current understanding and treatment of these effects are constrained by the limited tools available to monitor and manipulate neural dynamics within the spinal cord. Rodent models, given the relative ease of genetic manipulation, have become essential in neuroscience research. Optogenetic modulation of genetically targeted neuronal populations in the rodent spinal cord enables investigations of neural pathways critical to functional recovery after injury. Inspired by promising preclinical and early clinical studies that support spinal stimulation to aid rehabilitation following paralysis, recent research has focused on developing flexible, stretchable probes for optical and electrical stimulation on the surface of rodent spinal cords. The capability for simultaneous neural recording and stimulation will likely be critical for elucidating the electrophysiological mechanisms underlying functional recovery. Additionally, implantable devices designed for deeper spinal cord regions could allow for the interrogation of specific interneuronal populations, offering insights into their roles in connection loss and recovery following injury.

Using the COMPACT shrinking technique, a novel optrode design consisting of a single optical core encased in a thin, conductive layer of CNTs-PVA was developed. For in vivo validation, these optrodes were implanted into the L3 region of the spinal cord in VGAT-ChR2 mice, achieving a penetration depth of 300 microns. The conductive layer successfully recorded spontaneous intraspinal neural activity, distinguishing two unique single-unit waveforms. Simultaneously, blue light was delivered through the optical core to inhibit excitatory sensory neurons, with the resultant neural inhibition effectively tracked through electrophysiological signals in freely moving mice. This optrode, integrating optical stimulation and electrophysiological recording within a single device, offers a toolset for simultaneous neural modulation and monitoring, ideally suited to complex and dynamic in vivo experimental environments.

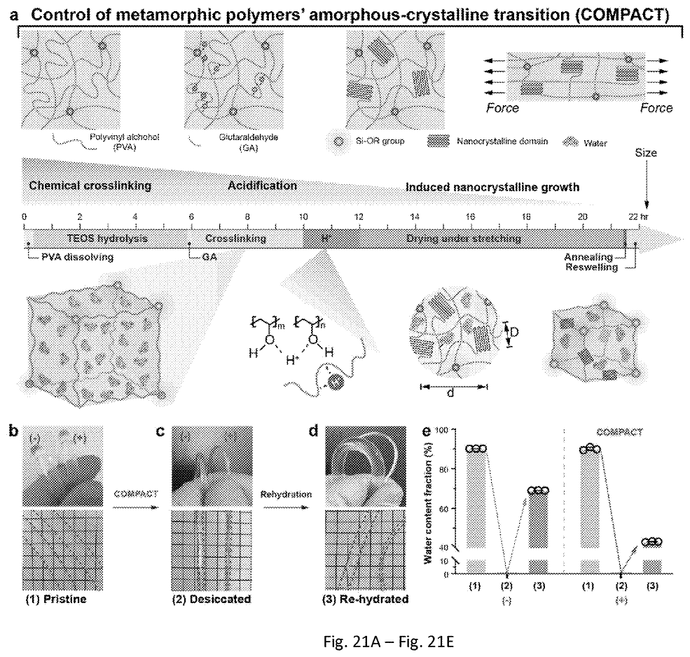

Controllable shrinking for hydrogel integration and miniaturization. Chemically cross-linked PVA hydrogels have been widely employed with superior optical properties³⁸, fatigue-resistance^{39, 40, 41} and biocompatibility for bioelectronics applications^{42, 43}. To further explore PVA hydrogels' controllable miniaturization properties while preserving these advantageous features, fabrication approaches were designed by control of metamorphic polymers' amorphous-crystalline transition with the following aspects: (i) polymer chains folding and immobilization with multiple cross-linkers, (ii) intervention on intermolecular chain interactions in the hydrogel matrix, (iii) inducing the oriented growth of nanocrystalline domains. The COMPACT strategy was implemented following three major procedures to control individual polymer chain folding, polymer chain network interactions and nanocrystalline growth. Hydrolysis of TEOS in PVA solutions was introduced through homogenization (FIG. 21A), followed by the addition of a generic cross-linker GA. A combination of two types of cross-linkers is chosen to allow the control of polymer chain mobility via covalent bonding and parallel tuning of hydrogels' refractive index. The cross-linked hydrogels were then acidified to promote intermolecular chain interactions. Fiber-shaped hydrogels were prepared via molding and extrusion methods. External mechanical stretching was applied to the fully acidified hydrogels and maintained during the desiccating process. After the removal of water molecules from hydrogels, high-temperature (100° C.) annealing was employed to further promote the growth and orientation of the nanocrystalline domains.

To test whether COMPACT strategy can preserve hydrogels volumetric shrinking under hydrated state, the dimensions and water fractions of cross-linked hydrogels under pristine, desiccated, and re-hydrated states (FIG. 21B-21E) were examined. At the pristine (FIG. 21B) and desiccated states (FIG. 21C), the two hydrogel fibers with TEOS-GA cross-linking (COMPACT+) and GA cross-linking (COMPACT−) exhibited comparable geometries and water fractions (FIG. 21E); however, only the TEOS-GA cross-linked PVA hydrogel fiber with acidification and mechanical stretching maintained the reduced diameters in the re-hydrated state (FIGS. 21D-21E).

Fabrication of miniaturized conductive hydrogel via COMPACT strategy. Given that CNTs are capable of percolating and forming anisotropic conductive pathways, combining them with the COMPACT shrinking technique enables the fabrication of thinner hydrogel microelectrodes with reduced mold sizes. To incorporate CNTs (length-to-diameter ratio of 2,000-10,000:1) into the PVA matrix, the same TEOS hydrolysis and mixing strategy was used. To prevent CNTs aggregation, the CNTs solution was dispersed in sodium dodecylbenzene sulfonate (SDBS) prior to mixing. The dispersed CNTs solution was then combined with PVA-TEOS-HCl and PVA-GA solutions at a weight ratio of 0.4:1:1 and subsequently degassed.

The CNTs-PVA hydrogel fibers were fabricated by following the standard fiber molding and extrusion procedures. For creating CNTs-PVA hydrogel electrodes, a CNTs-PVA hydrogel fiber was connected to a copper wire by dip-coating both components in silver paint. This dip-coated assembly was then inserted into elastic tubing (100 μm) to form a secure hydrogel-copper junction. To enhance conductivity at this junction, an additional thin layer of silver was applied, followed by sealing and reinforcement with UV-curable epoxy.

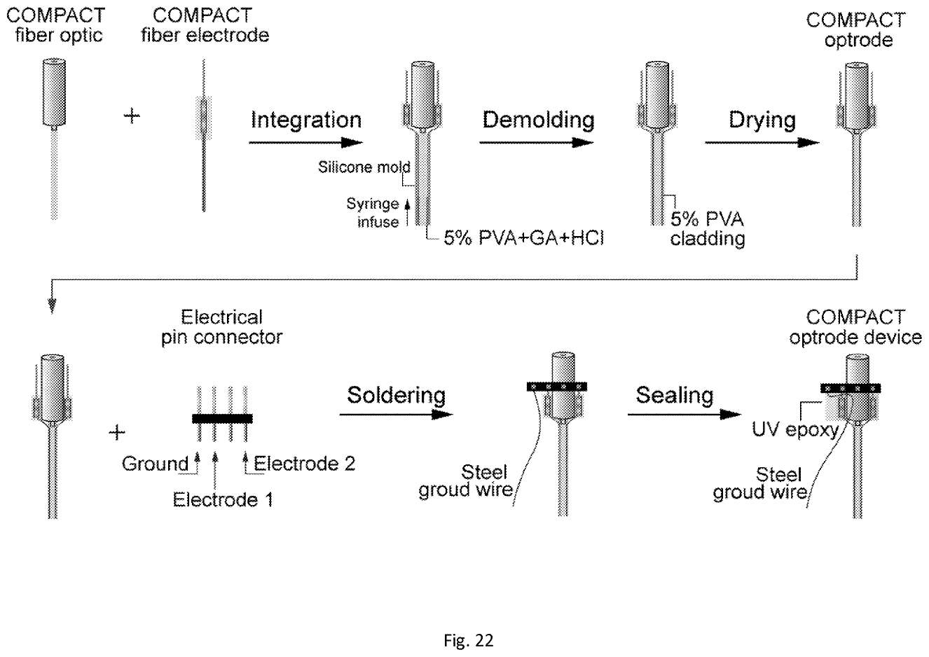

For the fabrication of complete COMPACT optrode devices, a four-pin connector was used to connect the two electrodes, while a 50 μm diameter steel wire served as the grounding element. To ensure structural integrity and reliable connections, UV-curable epoxy was applied to seal and reinforce the solder joints between the electrical pin connector and electrodes. This approach produces robust and precisely aligned optrode devices, designed to provide high-performance optical and electrical interfacing for advanced bioelectronic applications (FIG. 22).

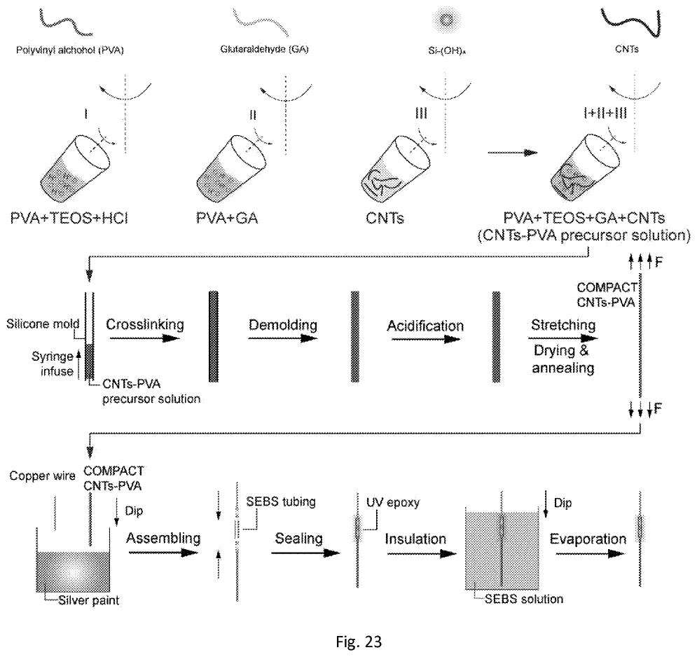

To insulate the CNT-PVA hydrogel fiber, a solution of SEBS (20 wt. % SEBS in toluene) was applied via dip-coating, forming a protective insulating layer upon solvent evaporation. This approach resulted in a fully fabricated CNTs-PVA hydrogel electrode with improved stability, conductivity, and insulation, optimized for applications in bioelectronic interfaces where flexibility and precise conductivity are essential (FIG. 23).

To fabricate COMPACT optrode fibers, a COMPACT fiber optic and two COMPACT fiber electrodes were positioned and aligned within a silicone mold (500 μm). A 5 wt. % solution of PVA with GA was prepared, followed by degassing and thorough mixing. Separately, a 5 wt. % solution of PVA with HCl was also degassed and mixed. These two solutions were then combined at a weight ratio of 1:1 and degassed. The resulting PVA-GA-HCl mixture was injected into the silicone mold and allowed to cross-link at room temperature for 4 hours, forming a stable cladding layer surrounding the optical fiber and electrodes.

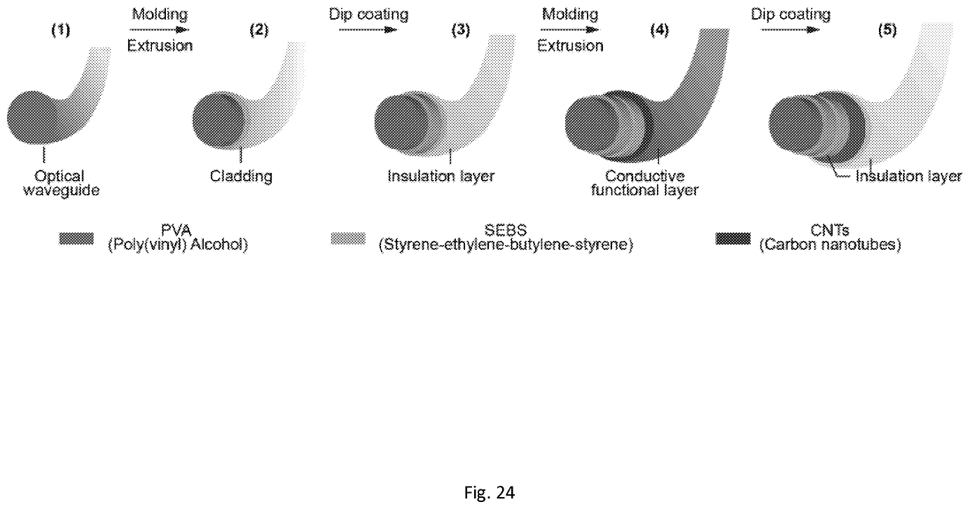

Dip coating of conductive functional layers. Dip coating provides a uniform, consistent layer along fibers, which is essential for applications demanding precise coating thickness, such as optical fibers and biomedical devices. It was hypothesized that utilizing dip coating to apply a thin layer of CNTs-PVA around an optical hydrogel probe would enable a uniform coating thickness, expand the effective electrical recording surface area for capturing larger neuronal populations, and enhance interfacial toughness through polymer chain infiltration (FIG. 24). This approach may improve both the functional integration and mechanical resilience of the probe within the neural tissue.

Hydrogel optrode fabrication via COMPACT strategy. To fabricate COMPACT optrode fibers, a COMPACT fiber optic, and two COMPACT fiber electrodes were inserted and aligned in a silicone mold (500 μm). GA was added into a 5 wt. % PVA solution and processed by degassing and mixing. HCl was added into a 5 wt. % PVA solution with degassing and mixing. The above two solutions were mixed (weight ratio=1:1) and degassed. The mixture of PVA-GA-HCl was injected into the silicone mold to cross-link (RT, 4 hours) and form a cladding layer around the optical fiber and electrodes. To fabricate COMPACT optrode devices, a four-pin electrical pin connector was used to connect two electrodes, and a steel wire (50 μm diameter) was used for grounding. UV epoxy was used to seal and reinforce the soldering junction between the electrical pin connector and electrodes.

Dimension measurements of hydrogel fibers. Microscopic images of hydrogel fibers were captured using a bright field mode microscope (AmScope) in MilliQ water. Three distinct regions of each fiber, namely two ends and the middle part, were imaged. The diameter of each fiber was measured using ImageJ software, with nine measurements taken for each fiber. The length of the fibers was measured using a caliper, with three measurements taken for each fiber.

SEM imaging. SEM was performed on dried samples using an FEI Magellan 400 XHR instrument. To analyze the cross-sectional morphology of the integrated hydrogel optrode probe, the probe was sectioned into thin pillars (0.1 mm in height) and subsequently mounted on carbon tape for imaging. For representative SEM images, each sample was repeated ten times with similar results.

TEM imaging. The TME images were acquired under a transmission electron microscope (FEI Tecnai 12). The carbon nanotubes were diluted (1:10) in MilliQ water and deposited on a copper grid (Sigma-Aldrich, FCF200-Cu) for imaging. For representative TEM images, each sample was repeated ten times with similar results.

Stability tests of hydrogel fibers. The fabricated COMPACT hydrogel fibers (3 wt. % TEOS) were incubated at 37° C. under physiological-like solutions (saline, ionic strength 305~310 mOsm, pH from 6.0 to 8.0) over 3 months to validate the stability of hydrogel materials. The dimensions of fiber were measured before and after the incubation and statistical analysis was performed on the dimensions between pre-incubation and post-incubation each week.

In vivo optrode device implantation into the mouse brain. Mice were anesthetized with 1.0% isoflurane and placed on a stereotactic frame (RWD Life Science) equipped with a heating pad to maintain body temperature. Surgery was conducted under sterile conditions, and 1% isoflurane was continuously administered to maintain anesthesia. Allen Brain Atlas was utilized to align the skull and establish optrode device coordinates (VTA, AP: −3.00 mm, ML: + (or −) 0.45 mm, DV: −4.80 mm) based on the mouse brain atlas. Prior to optrode implantation, a ground screw was implanted (AP: −3.50 mm, ML: −(or +) 1.50 mm, DV: −0.20 mm) and cerebrospinal fluid was contacted with the screw. The optrode devices were fixed on the skull with adhesive (Parkell, C&B METABOND) and reinforced with dental cement (Jet Set-4). Following the removal of isoflurane, the mice were monitored on the heating pad until fully awakened. Thy1::ChR2-EYFP mice (n=5) were implanted with optrode devices.

In vivo implantation in spinal cord. Mice were anesthetized under 1% isoflurane. Once a stable plane of anesthesia was reached, the area surrounding the dorsal hump was shaved and then cleansed with alternating applications of iodine and 75% alcohol. For implantations at spinal segment L3 or L4, make an incision starting slightly caudal of the peak of the dorsal hump, extending approximately 0.5 cm rostral and 0.5 cm caudal from the initial incision site. Larger incisions can be performed initially to allow for greater access. Make an incision medial to the white tendons running on either side of the vertebral column, spanning slightly more than one vertebral segment, from the outer edge of the caudal transverse process to outer edge of the rostral transverse process. Next, cut the tendon attached to the caudal transverse process, controlling bleeding using absorption spears. During this step, clear as much tissue from the transverse processes as possible, while being careful not to damage any nerves entering the spinal cord. Repeat this process on both sides of the spinal column. The vertebrae (T12 and T13) of interest were identified, and then a small incision was made between the tendons and the vertebral column on either side. All the tissue was removed from the surface of the bone. Then the spine was secured using customized spinal adaptor clamps. The tough layer of dura matter was poked with a hole using a 30Ga needle. The electrode bundle was cleaved to a length less than 500 μm and the electronic device was place on an implantation bar from the stereotaxic table. The device was then positioned in between vertebrae T12 and T13. The device then penetrated spinal cord tissues with a 300 μm depth. A small amount of sterile gel around the electrode bundle and over the surface of the bone was used to reduce the possibility of bone bleeding and secure the electrical pin in place. Next, the electrode pin was cemented in place using dental cement and, after the cement dried, sutured the skin surrounding the dental cement. Meloxicam and buprenorphine were administered subcutaneously. Mice were allowed to recover under a heat lamp before being returned to their cage.

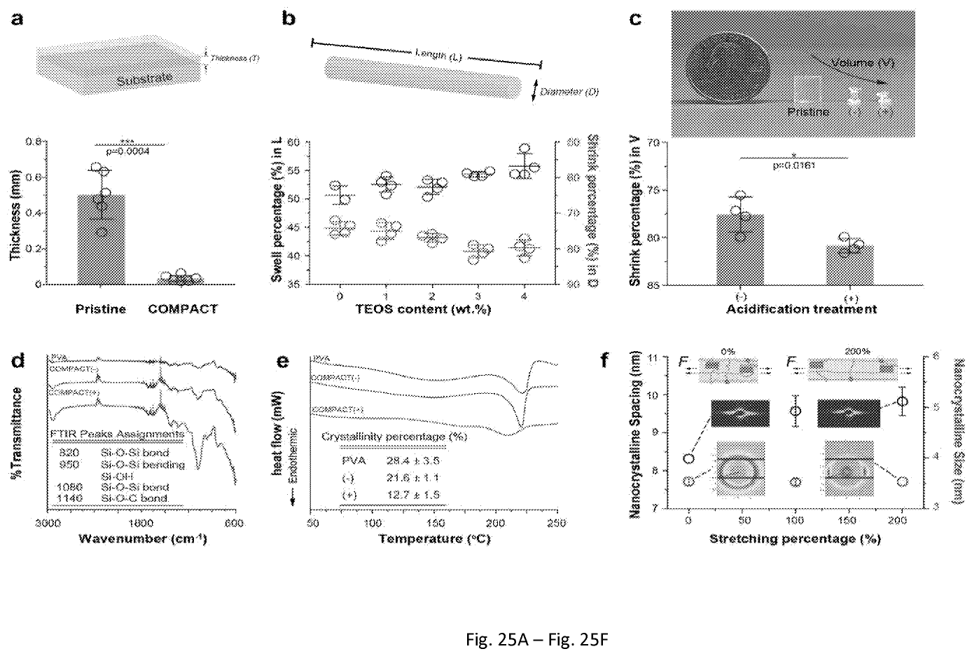

COMPACT strategy for hydrogels controllable shrinking. Hydrogels retained shrinking behaviors in the re-hydrated state with COMPACT treatment. Whether size reduction is dependent on the materials' geometries and external constraints tested. Hydrogels were prepared with the shapes of thin film, fiber, and block, and examined the changes of COMPACT hydrogel film thickness (T, FIG. 25A), fiber diameter (D, FIG. 25B) and volume (V, FIG. 25C). TEOS-GA cross-linked PVA hydrogel thin films with acidification treatment exhibited a thickness reduction ratio of 93.4±3.6% (pristine thickness: 501±134 μm; re-hydrated thickness: 33±18 μm, FIG. 1f). TEOS-GA cross-linked PVA hydrogel fibers, with treatment of acidification and mechanical strain (200%), reached the maximum diameter shrinking ratio of 79.7±2.3% (FIG. 1g). In 3D free shrinking structures, 80.9±0.7% volumetric shrinking in acidified TEOS-GA cross-linked cylinders (FIG. 25C) was observed.

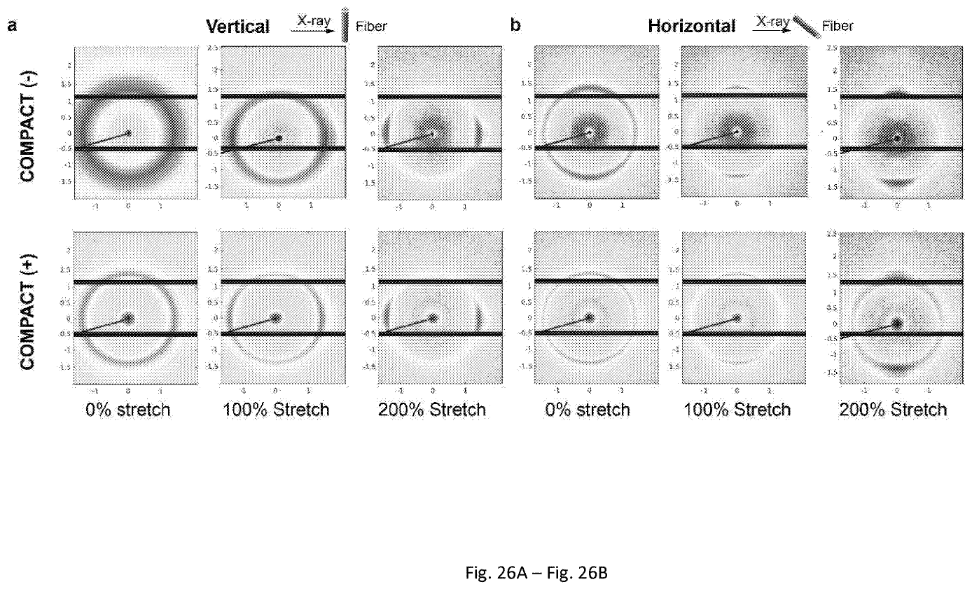

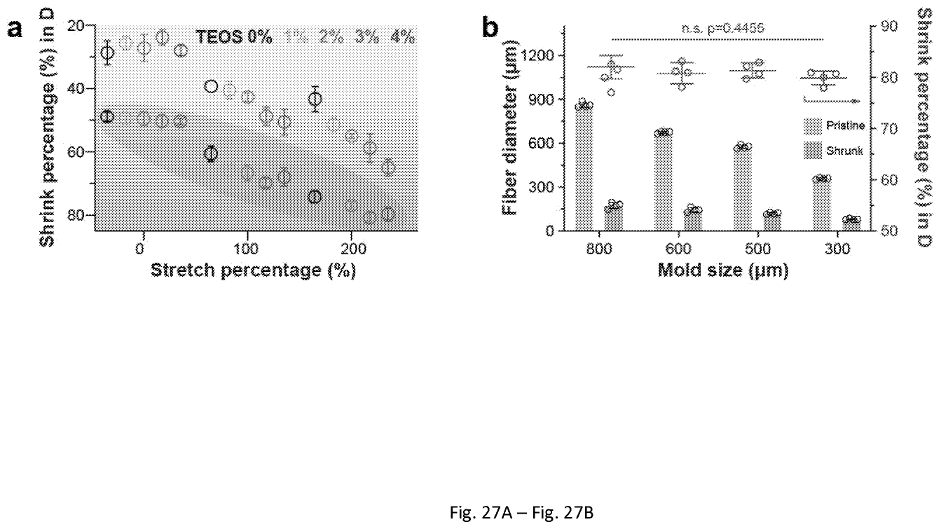

The mechanisms of the sustained hydrogel volumetric decrease and the design of amorphous and crystalline architectures were investigated. Fourier transform infrared spectroscopy (FTIR) results indicated covalent bonds (Si—O—Si and Si—OH) generated in the COMPACT hydrogel network (FIG. 25D). The Si—O—Si (1080 cm⁻¹) and Si—OH (950 cm⁻¹) bonds came from the hydrolyzed TEOS Si—OR groups' reactions with the hydroxyl groups on PVA chains. The generic cross-linker GA reactions were confirmed by the observation of C═O bond (1740 cm⁻¹) and the Si—O—C bond (1140 cm⁻¹) from the reaction with TEOS Si—OR groups. Besides confirming covalent bonds generated among hydrogel polymer chains, DSC results exhibited the change of polymer chain interactions and polymeric crystallinity after COMPACT treatment⁴⁴. PVA powders showed 28.4±3.5% crystallinity (FIG. 25E and Table 3), similar to the reported crystallinity percentage of semi-crystalline PVA polymers⁴⁵. GA-cross-linked PVA hydrogels exhibited 21.6±1.1% crystallinity while the additional TEOS cross-linking and acidification suppressed the polymer chain folding to form crystalline domains (crystallinity: 12.7±1.5%, Table 4). The nanocrystalline domains and orientation were observed with X-ray scattering techniques. The SAXS results suggested that the size of PVA nanocrystals was measured as 3.5±0.1 nm while the nanocrystalline spacing increased from 8.32±0.08 nm to 9.83±0.38 nm after 200% axial stretching (FIG. 25F and FIG. 26). WAXS 2D patterns suggested that the lamellae crystal domains were re-oriented along the axial stretching direction (FIG. 25F and FIG. 27). Similar anisotropic nanocrystalline structures have been reported in other PVA hydrogels⁴⁶.

TABLE 3
Crystallinity % calculation from DSC results

| | ΔHₘ | ΔH_{f} | Cry % = ΔHₘ/ |
|---|---|---|---|
| | (mJ/mg) | (mJ/mg) | ΔH₁₀₀ · 100 (%) |
| PVA | 40.9 ± 20.6 | 150 | 28.4 ± 3.5 |
| COMPACT (−) | 32.4 ± 1.6 | 150 | 21.6 ± 1.1 |
| COMPACT (+) | 19.1 ± 2.2 | 150 | 12.7 ± 1.5 |

TABLE 4
Crystallinity % calculation from DSC results

| | ΔHₘ | ΔH_{f} | Cry % = ΔHₘ/ |
|---|---|---|---|
| | (mJ/mg) | (mJ/mg) | ΔH₁₀₀ · 100 (%) |
| 0% TEOS | 32.4 ± 1.6 | 150 | 21.6 ± 1.1 |
| 3% TEOS | 24.1 ± 1.5 | 150 | 16.1 ± 1.0 |
| 4% TEOS | 19.1 ± 2.2 | 150 | 12.7 ± 1.5 |

In COMPACT hydrogels, chemical cross-linking and acidification treatment both contribute to the retained volumetric decrease upon re-hydration while mechanical deformation induces the orientated nanocrystalline growth. An increased number of chemical cross-linkers, TEOS (0-4 wt. %), enhanced the anchoring of amorphous PVA chains through covalent cross-linking and prevented swelling in the hydrated state. Under the same cross-linking degree, acidification treatment granted polymer chains enhanced interactions and suppressed the folding of polymer chains to form crystalline. Nanocrystalline domains maintained the nanoscale size (~3.5 nm) without compromising the light transmittance in the visible range. Axial mechanical deformation introduced tensile stress to re-orientate polymer chain alignment and created anisotropic nanostructures, which enabled hydrogel fibers' desired decrease in diameter while causing a minimal effect on crystallinity degree or nanocrystalline size.

Controllable hydrogel shrinking through cross-linking and polymer chain crystallization process provides an effective methodology to miniaturize hydrogel bioelectronics, especially for the application in vivo. The approaches to regulating polymer chain folding and interactions can be extended to other semi-crystalline polymers. Without affecting the nanocrystalline size, the mechanical stretching method offers a straightforward way to create anisotropic orientations of polymeric nanostructures. The molding and extrusion approaches offer a series of precisely controlled hydrogel fiber diameters with structural homogeneity and low surface asperity to avoid diffuse reflection at the hydrogel interfaces. Although the mold sizes are commercially limited, COMPACT procedures, including regulating polymer and crosslinker constituent content and fiber extensions can expand the range of available fiber sizes.

Controllable hydrogel fiber fabrication and its properties. With COMPACT-enabled hydrated hydrogel size reduction, this methodology was expanded to develop a series of hydrogel fibers with controlled diameters and tunable optical and mechanical properties for biomedical use. A rational and comprehensive shrinking diagram was mapped by varying the content of inorganic cross-linker (TEOS), acidification, and external mechanical stretching (FIG. 27A). Generally, increasing cross-linking density with more cross-linkers yielded less ductile polymer chains with reduced dimension upon hydration. Acidification treatment dramatically boosted shrinking percentages across different cross-linking densities while mechanical static stretching further decreased hydrogel fibers in diameters (79.7±2.3%). To fit COMPACT into a practical molding-extrusion fabrication process, a series of hydrogel fibers made with different sizes of silicone molds (FIG. 27B) was examined. Independent from the mold size, all COMPACT hydrogel fibers reached reduced diameters of more than 79%, which is consistent with the shrinking diagram. As an example, using 300 μm (inner diameter, ID) silicone molds, thin hydrogel fibers were fabricated with diameters of 80±4 μm.

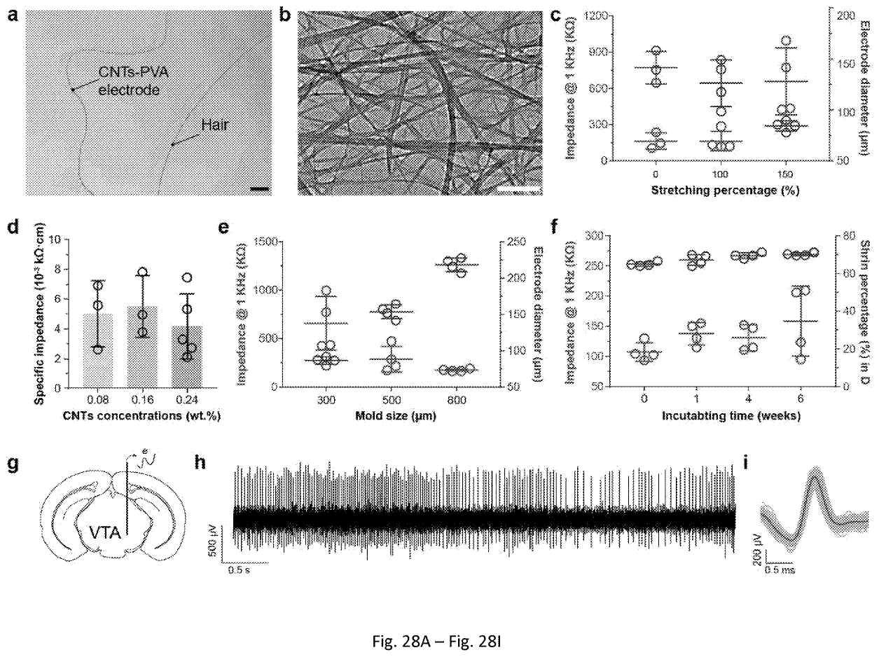

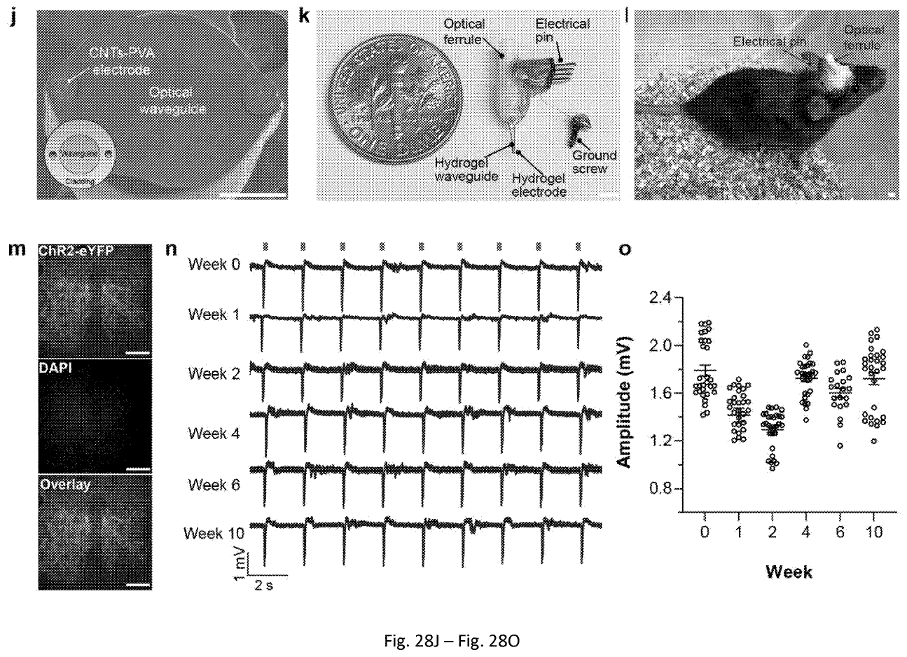

Multifunctional hydrogel neural probes with two electrodes. Hydrogel matrix can incorporate various nanoscale materials to extend the functionalities while maintaining stretchability. To enrich hydrogel neural probes' modality for electrical recordings, conductive CNTs (length to diameter ratio 2,000-10,000:1) were introduced into PVA hydrogels during hydrogel cross-linking (FIG. 28A-28B). Through acidification to promote polymer chain interactions and mechanical stretching facilitated CNTs plaiting into polymer matrices and ensured entanglement with PVA chains and consequently augmented electrical conductivity as a percolated network^{63,64}. After introducing CNTs (0.08-0.24 wt. %), insignificantly changed nanocrystallinity but decreased nanocrystalline sizes were observed. These results could be interpreted as the limited polymer chain folding under additional interactions between nanomaterials and polymer chains. When CNTs-PVA hydrogel fibers underwent mechanical stretching, they preserved anisotropic structures with similar sizes. With rigid carbon materials incorporated into the hydrogel matrix, the CNTs-PVA composite exhibited an increase in elastic modulus (0.16 wt. % CNTs-PVA hydrogels, 39.4±13.7 MPa), and decreased stretchability (47.9±12.2%). For the use as an electrode in vivo, the CNT concentration was optimized to balance the conductivity and mechanical properties. CNTs-PVA hydrogel electrodes (86±5 μm diameter, FIG. 28C), insulated with a viscoelastic coating of styrene-ethylene-butylene-styrene (SEBS), exhibited impedances of 658±277 kΩ at 1 kHz and impedance was tunable with designed mold sizes to control the electrode diameters and CNTs loadings (FIG. 28D, 28E). The stability of CNTs-PVA electrodes was evaluated through over 6 weeks of incubation in PBS (37° C., FIG. 28F).

Instead of recording collective electrical response from muscles, CNTs-PVA hydrogel electrodes were implanted in mouse VTA to record neuron spontaneous spiking activity in anesthetized wild-type mice under continuous isoflurane (FIG. 28G-28I). A bandpass filter of 300-3000 Hz was applied to detect spiking activity, and one distinct cluster of spikes was found by principal component analysis (PCA). The signal-to-noise ratio (SNR) of these spiking activities was approximately 3.73 with repeatable waveforms.

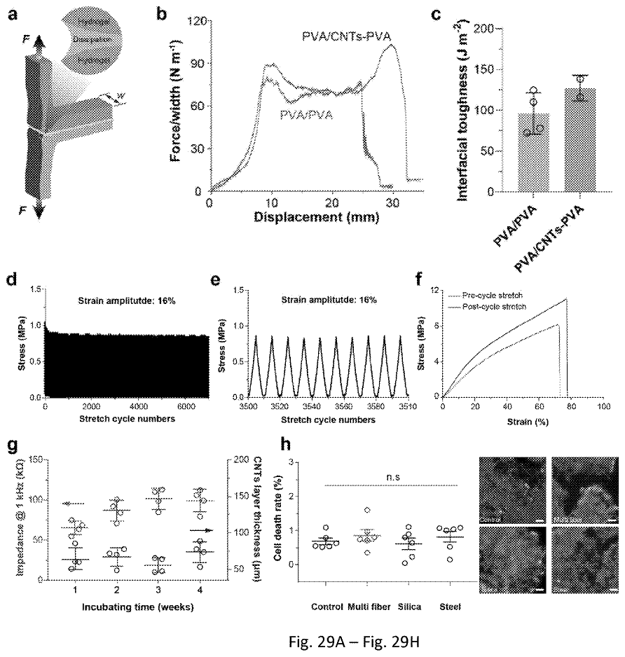

When extending hydrogel miniaturization from bulk materials to interfaces, the COMPACT strategy offers an opportunity for multiple components integration. Since RI-distinct core-cladding structures ensure light transmission in optical cores, two CNTs-PVA electrodes were introduced into the cladding layers with a COMPACT hydrogel core (FIG. 28J). A hydrogel optoelectrical device termed optrode (FIG. 28J), is designed to enable optical modulation with simultaneous electrophysiological recording. In Thy1::ChR2-EYFP mice, blue light pulses (2=473 nm, 0.5 Hz, pulse width 50 ms, 10 mW/mm²), delivered through the hydrogel optical core, consistently activated ChR2-expressing neurons in VTA while the neural electrical signals were collected through CNTs-PVA electrodes (FIG. 28L-29M). The optical evoked potentials were repeatedly captured with correlation with the onset of light stimulation over 10 weeks post-implantation (FIG. 28N-28O).

Simultaneous bi-directional stimulation and recording of neurons with optical and electrical modalities offer comprehensive approaches to studying brain function. The COMPACT strategy offers the convenience of integrating multiple functional components into a single miniaturized device. Successive rounds of molding with strong polymer chain infiltration at the interfaces enable the design of multimodal microstructures. Currently, the number of integrated components, such as electrodes and microfluidic channels, is limited by coaxial alignment in the secondary molding step; the accessibility and throughput of multimodal fabrication can be further improved with guiding devices to facilitate integration and alignment, or alternative coating approaches.

Multifunctional hydrogel neural probes with conductive layer. To evaluate the interfacial toughness between the conductive CNTs-PVA layer and the hydrogel optical fiber core, 180° peel tests were performed. Initially, the interfacial toughness of PVA-PVA was examined as a control (FIG. 29A-29C) and a toughness value of 96 J/m² observed, which is higher than previously reported values⁸⁵. This enhanced toughness may be attributed to nanocrystalline growth within the PVA hydrogel matrix and the use of high molecular weight PVA, which facilitates polymer chain infiltration. With the incorporation of CNTs, the interfacial toughness between CNTs-PVA and PVA further increased, likely due to improved mechanical interlocking and adhesion provided by the CNTs, enhancing the composite's structural integrity (127 J/m², FIG. 29C).

To assess the mechanical stability and fatigue resistance of the multifunctional fibers, there were subjected to over 6,000 cycles of mechanical stretching at a 16% strain amplitude and compared their mechanical properties before and after cyclic loading (FIG. 29D-29F). The results indicate that the fibers maintained their structural integrity and mechanical performance over repeated deformation cycles. For electrochemical stability, the multifunctional probes were incubated in PBS at 37° C. for four weeks, and impedance measurements demonstrated consistent performance over time, suggesting sustained stability in physiological conditions (FIG. 29G). Cytotoxicity tests further confirmed the biocompatibility of the multifunctional probes, showing low cell death rates upon contact with cells (FIG. 29H), underscoring the potential of these devices for safe and long-term biological interfacing.

To further evaluate the functionality and feasibility of the conductive layer, ring-shaped optrode devices were implanted into the L3 spinal cord region of WT mice. Using the setup described above, spontaneous neural activity was successfully recorded in conscious mice. Spike sorting analysis enabled the differentiation of two distinct single-unit waveforms, confirming the device's ability to capture and resolve separate neuronal signals. These results validate the conductive layer's efficacy in recording neural activity with high fidelity, supporting its potential for use in complex in vivo neural monitoring applications.

To evaluate the multifunctionality of the ring-shaped optrode, it was implanted into VGAT-ChR2 mice, a model in which inhibitory neurons ChR2 can be optogenetically activated. VGAT-ChR2 mice are valuable for pain management studies as activating inhibitory neurons may modulate pain pathways by suppressing excitatory neural activity. The optrode was implanted in the y4 sensory and motor neurons of the L3 spinal cord region, allowing for concurrent monitoring of electrophysiological activity during light delivery.

With blue light stimulation, spontaneous neural activity was suppressed, as evidenced by a reduction in recorded spike events. Single-unit analysis indicated the presence of one active neuron during optical stimulation. In contrast, in the absence of optical stimulation, spontaneous neural activity increased, with a higher frequency of spike events. Furthermore, single-unit sorting revealed a second distinct waveform, suggesting the activation of additional neuronal populations. These results demonstrate that the optrode effectively integrates optical stimulation and electrophysiological recording, providing a powerful tool for studying neuromodulation and neural dynamics in vivo. Compared to spontaneous activities without optical stimulation, the neural activities came back with more firings and another wave form from the single unit sorting observed.

A set of hydrogel cross-linking chemistry and fiber-shaped device microfabrication approaches were developed through a bottom-up strategy of tuning polymers' amorphous-crystalline transition for hydrogel bioelectronics miniaturization and integration. The COMPACT strategy provides an accessible, scalable, and controllable fabrication method for micro-structured hydrogel fibers with consistently low asperity. These hydrogels provide a platform for functionally augmented interfaces through loadings of additional nanomaterials. COMPACT hydrogels can be further designed into step-index optical probes and optoelectronic devices which are well-suited for neural modulation and recordings concurrent with behavioral assays in mice.

The COMPACT strategy is generalizable for soft and stretchable bioelectronics. Polymer matrices provide sufficient free volume for water access as well as nanomaterials' incorporation. High aspect-ratio nanomaterials can be effectively entangled with polymer chains through cross-linking and condensation during acidification and stretching. This procedure augments electrical conductivity while maintaining viscoelasticity. Compared to other soft bioelectronics fabrication approaches, such as lithography and micro-printing, COMPACT technique offers scalable and efficient multimodal hydrogel fibers manufacturing without the need for expensive and sophisticated facilities. COMPACT multifunctional hydrogel neural probes have been employed for bi-directional optical interrogation concomitant with mouse social behaviors and electrical recordings of light-triggered neural activity in mice. Extended functionalities, such as drug or viral vector delivery, can be further achieved by integrating additional microfluidic channels in the cladding layer and retaining light transmission efficiency in the optical core. COMPACT multifunctional neural probes involve independent component alignment and miniaturization steps, which potentiates the integration of multiple components with various lengths to target multiple depths of tissue within single-step implantation. This adaptability will increase the density of functional interfaces and overcome the traditional limitation of fiber-shaped neural probes with single-target interfaces at the tip.

Control over semi-crystalline polymers' amorphous-crystalline transition creates a direct fabrication methodology for elastic soft materials. Extending it to the manufacture of sophisticated optoelectronic devices, the COMPACT strategy imparts a generalizable and modular platform for hydrogel bioelectronics' miniaturization and integration, which consequently enables multimodal interrogation of complex biological systems.

The development of multifunctional hydrogel neural interfaces remains limited by the lack of integrated capabilities required for advanced neuromodulation. The present technology focuses on two critical areas: (1) designing hydrogel neural probes with optical, electrical, mechanical, and/or chemical functionalities, and (2) achieving integration and miniaturization of these components into cohesive, multifunctional hydrogel-based neural probes.

A hydrogel-based optical fiber with high refractive index, fatigue resistance, low bending stiffness, and long-term structural and functional stability, enabling reliable optical stimulation and recording capabilities is provided.

Hydrogel bioelectronic devices are provided capable of interfacing with deep brain regions and the spinal cord, facilitating the monitoring of endogenous neural activity, delivering therapies, and contributing to pain management strategies within the spinal cord.

Micro-patterned hydrogels optimized for controlled component delivery were developed, advancing precision in therapeutic and diagnostic applications.

Engineered hydrogel chemistries tailored for miniaturization are provided, allowing for the integration of multiple functionalities within compact neural probes.

Hydrogel bioelectronics. Hydrogel-based bioelectronics are increasingly utilized for skin-attachable, implantable, and semi-implantable sensing applications, offering critical advancements in monitoring human motion, analyzing biomarker levels in bodily fluids, and recording bioelectric signals via flexible electrodes²⁴⁵. These systems effectively address many limitations of conventional rigid health monitoring devices, enhancing comfort and portability. Despite considerable progress in hydrogel-based bioelectronics for health monitoring, significant challenges remain.

One primary issue is the manufacturing process of hydrogels, as their molecular structure inherently contributes to substantial thickness, hindering miniaturization and multifunctional integration. Scaling down hydrogel-based devices can compromise mechanical stability and structural integrity. Additionally, hydrogels are soft and hydrated, posing limitations on manufacturing precision and making it difficult to create high-accuracy structures. Issues such as water loss or swelling further complicate miniaturization by altering device performance and dimensions. Therefore, new manufacturing techniques have been proposed to enhance the integration and multifunctionality of hydrogel-based bioelectronics, while still leveraging their inherent flexibility and biocompatibility.

The application of hydrogel-based bioelectronics in real-time disease monitoring and targeted diagnostics and therapeutics presents a compelling opportunity. In health monitoring, the choice and specificity of biomarkers are crucial, as overlapping biomarker profiles among different diseases can reduce diagnostic accuracy. Additionally, low biomarker concentrations necessitate sensitive detection methods to enhance signal specificity.

Hydrogel optical fibers. The hydrogels need not include only non-functionalized monomers and polymers, and rather may be modified either from particular monomers having required functional groups²⁴⁶ and/or through chemical modification after the hydrogel is formed. Functionalization of these polymers provides opportunities for specific sensing and/or therapeutic intervention.

The integration of modern nanotechnologies with optical fiber systems represents an emerging advancement in biosensing technology, enabling biological detection and optical manipulation of biospecimens, such as organic nanoparticles, biomolecules, and bacterial viruses, via traditional silica-based optical fibers²⁴⁷. New designs involving micro- and nano-optical fibers, including tapered fibers, plasmonic fibers, microlens-equipped fibers, and nanowires, have ben proposed, which may be compatible for inclusion with the present fibers. These may provide low optical loss, strong optical confinement, and large evanescent fields, for high-sensitivity, high-resolution optical sensing²⁴⁸ Moreover, there is potential to fabricate these fibers from biocompatible and bioresorbable materials, which would support minimally invasive applications and limit cytotoxicity.

Hydrogel as chemical/drug/pharmacy delivery platforms. Hydrogels offer a promising platform for the delivery of therapeutic agents across a range of disorders, including ocular infections, cancer, rheumatoid arthritis, and fungal infections. Recent innovations in hydrogel design have focused on enhancing mechanical strength, thereby addressing limitations of traditional hydrogels and expanding the potential of smart hydrogels in applications requiring controlled swelling and improved mechanical properties. These enhancements allow for better dimensional compatibility with target tissues and organs. Additionally, advances in biocompatibility enable hydrogels to mimic the extracellular matrix (ECM) more accurately, replicating both its structure and function.

Hydrogels may have tunable degradation rates to align with the mechanical properties and regenerative demands of the targeted tissue. This functionality may be achieved through use of inherently biodegradable gels, inclusion of a sufficient percentage of monomers that form labile polymers to ensure loss of mechanical integrity at the desired rate, and/or inclusion of components or treatments that actively degrade the gel matrix. The technology may also be used to develop artificial organs, for example electro-stimulatory organs, electroresponsive organs, artificial endocrine, paracrine, or exocrine glands, muscular organs, etc. Hydrogels are available for controlled drug delivery, allowing for the development of hydrogel-based vehicles that could overcome limitations of conventional delivery methods and deliver therapeutics more effectively for a variety of diseases. A multifunctional drug delivery vehicle is specifically contemplated as an application of the technology.

Beyond brain applications, hydrogels demonstrate significant potential for interfacing with the heart, vagus nerve, and gastrointestinal tract. By leveraging their adhesive properties, hydrogel-based optical fibers can be affixed to cardiac tissue for optogenetic stimulation without suturing, enabling precise control over cardiac rhythm, which holds promise for treating arrhythmias and other heart conditions. Similarly, cuff-mounted optical fibers applied to the vagus nerve may aid in modulating autonomic control over various organ systems, potentially assisting in the regulation of inflammation, metabolism, and mood.

Furthermore, understanding and modulating the gut-brain axis, which is implicated in conditions such as sugar and alcohol addiction, requires neuromodulatory capabilities within both the gastrointestinal system and the brain. Hydrogel devices offer a minimally invasive approach to achieve this, enhancing potential treatment options. Additionally, for patients with spinal cord injuries who experience challenges in bladder control, hydrogel interfaces applied to the bladder could provide targeted electrical or optical stimulation, facilitating urination control and improving quality of life. These advancements position hydrogels as a versatile platform for a new generation of bioelectronic devices and neuromodulatory therapies across diverse organ systems.

Thus, have been shown various embodiments, Further modifications, combinations, subcombinations, and permutations of the disclosed technology will be apparent to those of ordinary skill in the art.

### REFERENCES (EACH OF WHICH IS EXPRESSLY INCORPORATED HEREIN BY REFERENCE)

1. Frank, J.A., Antonini, M.-J. & Anikeeva, P. Next-generation interfaces for studying neural function. Nature Biotechnology 37, 1013-1023 (2019).
2. Tau, G.Z. & Peterson, B.S. Normal Development of Brain Circuits. Neuropsychopharmacology 35, 147-168 (2010).
3. Barnett, M. W. & Larkman, P. M. The action potential. Practical Neurology 7, 192 (2007).
4. Greengard, P. The Neurobiology of Slow Synaptic Transmission. Science 294, 1024-1030 (2001).
5. Sabatini, B. L. & Regehr, W. G. Timing Of Synaptic Transmission. Annual Review of Physiology 61, 521-542 (1999).
6. Maffeo, C., Bhattacharya, S., Yoo, J., Wells, D. & Aksimentiev, A. Modeling and Simulation of Ion Channels. Chemical Reviews 112, 6250-6284 (2012).
7. Yizhar, O., Fenno, Lief E., Davidson, Thomas J., Mogri, M. & Deisseroth, K. Optogenetics in Neural Systems. Neuron 71, 9-34 (2011).
8. Kandel, E. R. et al. Principles of neural science, Vol. 4. (McGraw-hill New York, 2000).
9. Freund, H. J. Motor unit and muscle activity in voluntary motor control. Physiological Reviews 63, 387-436 (1983).
10. Curti, S. & O'Brien, J. Characteristics and plasticity of electrical synaptic transmission. BMC Cell Biology 17, 13 (2016).
11. Millhorn, D. E. et al. Cellular and molecular mechanisms of chemical synaptic transmission. American Journal of Physiology-Lung Cellular and Molecular Physiology 257, L289-L310 (1989).
12. Kress, G. J. & Mennerick, S. Action potential initiation and propagation: Upstream influences on neurotransmission. Neuroscience 158, 211-222 (2009).
13. Bean, B. P. The action potential in mammalian central neurons. Nature Reviews Neuroscience 8, 451-465 (2007).
14. Connors, B. W. & Gutnick, M. J. Intrinsic firing patterns of diverse neocortical neurons. Trends in Neurosciences 13, 99-104 (1990).
15. Johnston, D., Magee, J. C., Colbert, C. M. & Christie, B. R. Active properties of neuronal dendrites. Annual review of neuroscience 19, 165-186 (1996).
16. Magee, J. C. & Johnston, D. Plasticity of dendritic function. Current Opinion in Neurobiology 15, 334-342 (2005).
17. Clark, B. A., Monsivais, P., Branco, T., London, M. & Hausser, M. The site of action potential initiation in cerebellar Purkinje neurons. Nature Neuroscience 8, 137-139 (2005).
18. Kampa, B. M., Letzkus, J. J. & Stuart, G. J. Dendritic mechanisms controlling spike-timing-dependent synaptic plasticity. Trends in Neurosciences 30, 456-463 (2007).
19. Gasser, H. S. & Erlanger, J. The Role Played By The Sizes Of The Constituent Fibers Of A Nerve Trunk In Determining The Form Of Its Action Potential Wave. American Journal of Physiology-Legacy Content 80, 522-547 (1927).
20. Coombs, J., Curtis, D. & Eccles, J. The interpretation of spike potentials of motoneurones. The Journal of Physiology 139, 198 (1957).
21. Dodge, F. A. & Cooley, J. W. Action Potential of the Motorneuron. IBM Journal of Research and Development 17, 219-229 (1973).
22. Colbert, C. M. & Pan, E. Ion channel properties underlying axonal action potential initiation in pyramidal neurons. Nature Neuroscience 5, 533-538 (2002).
23. Wollner, D. A. & Catterall, W. A. Localization of sodium channels in axon hillocks and initial segments of retinal ganglion cells. Proceedings of the National Academy of Sciences 83, 8424-8428 (1986).
24. Swadlow, H. A., Kocsis, J. D. & Waxman, S. G. Modulation of impulse conduction along the axonal tree. Annual review of biophysics and bioengineering 9, 143-179 (1980).
25. Waxman, S. G. Axonal conduction and injury in multiple sclerosis: the role of sodium channels. Nature Reviews Neuroscience 7, 932-941 (2006).
26. Naundorf, B., Wolf, F. & Volgushev, M. Unique features of action potential initiation in cortical neurons. Nature 440, 1060-1063 (2006).
27. Kole, M. H. P. et al. Action potential generation requires a high sodium channel density in the axon initial segment. Nature Neuroscience 11, 178-186 (2008).
28. Scanziani, M. & Häusser, M. Electrophysiology in the age of light. Nature 461, 930-939 (2009).
29. Tsukamoto, H. & Furutani, Y. in Optogenetics: Light-Sensing Proteins and Their Applications in Neuroscience and Beyond. (eds. H. Yawo, H. Kandori, A. Koizumi & R. Kageyama) 73-88 (Springer Singapore, Singapore; 2021).
30. Deisseroth, K. et al. Next-Generation Optical Technologies for Illuminating Genetically Targeted Brain Circuits. The Journal of Neuroscience 26, 10380 (2006).
31. Deisseroth, K. Optogenetics. Nature Methods 8, 26-29 (2011).
32. Deisseroth, K. Optogenetics: 10 years of microbial opsins in neuroscience. Nature Neuroscience 18, 1213-1225 (2015).
33. Grienberger, C. & Konnerth, A. Imaging Calcium in Neurons. Neuron 73, 862-885 (2012).
34. Thestrup, T. et al. Optimized ratiometric calcium sensors for functional in vivo imaging of neurons and T lymphocytes. Nature Methods 11, 175-182 (2014).
35. Zhang, Y. et al. Fast and sensitive GCaMP calcium indicators for imaging neural populations. Nature 615, 884-891 (2023).
36. Tong, Q. et al. Regulation of the Transient Receptor Potential Channel TRPM2 by the Ca<sup>2+</sup>Sensor Calmodulin *. Journal of Biological Chemistry 281, 9076-9085 (2006).
37. Zhang, Y.-P. & Oertner, T. G. Optical induction of synaptic plasticity using a light-sensitive channel. Nature Methods 4, 139-141 (2007).
38. Airan, R. D. et al. Integration of light-controlled neuronal firing and fast circuit imaging. Current Opinion in Neurobiology 17, 587-592 (2007).
39. Wilson, N. R., Runyan, C. A., Wang, F. L. & Sur, M. Division and subtraction by distinct cortical inhibitory networks in vivo. Nature 488, 343-348 (2012).
40. Fajardo, O., Zhu, P. & Friedrich, R. W. Control of a specific motor program by a small brain area in zebrafish. Frontiers in Neural Circuits 7 (2013).
41. Akerboom, J. et al. Genetically encoded calcium indicators for multi-color neural activity imaging and combination with optogenetics. Frontiers in Molecular Neuroscience 6 (2013).
42. Zhuang, Z. & Ho, H. P. Application of digital micromirror devices (DMD) in biomedical instruments. Journal of Innovative Optical Health Sciences 13, 2030011 (2020).
43. Anselmi, F., Ventalon, C., Bègue, A., Ogden, D. & Emiliani, V. Three-dimensional imaging and photostimulation by remote-focusing and holographic light patterning. Proceedings of the National Academy of Sciences 108, 19504-19509 (2011).
44. Nikolenko, V., Poskanzer, K. E. & Yuste, R. Two-photon photostimulation and imaging of neural circuits. Nature Methods 4, 943-950 (2007).
45. Minev, I. R. et al. Electronic dura mater for long-term multimodal neural interfaces. Science 347, 159-163 (2015).
46. Jeong, J.-W. et al. Wireless Optofluidic Systems for Programmable In& #xa0; Vivo Pharmacology and Optogenetics. Cell 162, 662-674 (2015).
47. Rössger, K., Charpin-El Hamri, G. & Fussenegger, M. Reward-based hypertension control by a synthetic brain-dopamine interface. Proceedings of the National Academy of Sciences 110, 18150-18155 (2013).
48. Shemesh, O. A. et al. Temporally precise single-cell-resolution optogenetics. Nature Neuroscience 20, 1796-1806 (2017).
49. Berlin, S. et al. Photoactivatable genetically encoded calcium indicators for targeted neuronal imaging. Nature Methods 12, 852-858 (2015).
50. Tischbirek, C., Birkner, A., Jia, H., Sakmann, B. & Konnerth, A. Deep two-photon brain imaging with a red-shifted fluorometric Ca2+indicator. Proceedings of the National Academy of Sciences 112, 11377-11382 (2015).
51. Deal, P. E., Kulkarni, R. U., Al-Abdullatif, S. H. & Miller, E. W. Isomerically Pure Tetramethylrhodamine Voltage Reporters. Journal of the American Chemical Society 138, 9085-9088 (2016).
52. Wang, J., He, T. & Lee, C. Development of neural interfaces and energy harvesters towards self-powered implantable systems for healthcare monitoring and rehabilitation purposes. Nano Energy 65, 104039 (2019).
53. Budday, S. et al. Mechanical characterization of human brain tissue. Acta Biomaterialia 48, 319-340 (2017).
54. Canales, A. et al. Multifunctional fibers for simultaneous optical, electrical and chemical interrogation of neural circuits in vivo. Nature Biotechnology 33, 277-284 (2015).
55. Xiang, Z. et al. Progress of Flexible Electronics in Neural Interfacing-A Self-Adaptive Non-Invasive Neural Ribbon Electrode for Small Nerves Recording. Advanced Materials 28, 4472-4479 (2016).
56. Wang, J., Wang, H., Thakor, N. V. & Lee, C. Self-Powered Direct Muscle Stimulation Using a Triboelectric Nanogenerator (TENG) Integrated with a Flexible Multiple-Channel Intramuscular Electrode. ACS Nano 13, 3589-3599 (2019).
57. Polanía, R., Nitsche, M. A. & Ruff, C. C. Studying and modifying brain function with non-invasive brain stimulation. Nature Neuroscience 21, 174-187 (2018).
58. Jiang, Y. et al. Rational design of silicon structures for optically controlled multiscale biointerfaces. Nature Biomedical Engineering 2, 508-521 (2018).
59. Campbell, P. K., Jones, K. E., Huber, R. J., Horch, K. W. & Normann, R. A. A silicon-based, three-dimensional neural interface: manufacturing processes for an intracortical electrode array. IEEE Transactions on Biomedical Engineering 38, 758-768 (1991).
60. Wise, K. D. & Najafi, K. Microfabrication Techniques for Integrated Sensors and Microsystems. Science 254, 1335-1342 (1991).
61. Liu, J. et al. Syringe-injectable electronics. Nature Nanotechnology 10, 629-636 (2015).
62. Jun, J. J. et al. Fully integrated silicon probes for high-density recording of neural activity. Nature 551, 232-236 (2017).
63. Vöröslakos, M. et al. Direct effects of transcranial electric stimulation on brain circuits in rats and humans. Nature Communications 9, 483 (2018).
64. Lu, C. et al. Flexible and stretchable nanowire-coated fibers for optoelectronic probing of spinal cord circuits. Science Advances 3, e1600955.
65. Tyler, D. J. & Durand, D. M. Chronic Response of the Rat Sciatic Nerve to the Flat Interface Nerve Electrode. Annals of Biomedical Engineering 31, 633-642 (2003).
66. Xiang, Z. et al. in 2015 37th Annual International Conference of the IEEE Engineering in Medicine and Biology Society (EMBC) 3157-3160 (2015).
67. Lee, S. et al. Toward Bioelectronic Medicine-Neuromodulation of Small Peripheral Nerves Using Flexible Neural Clip. Advanced Science 4, 1700149 (2017).
68. Boretius, T. et al. A transverse intrafascicular multichannel electrode (TIME) to interface with the peripheral nerve. Biosensors and Bioelectronics 26, 62-69 (2010).
69. Clark, G. A., Ledbetter, N. M., Warren, D. J. & Harrison, R. R. in 2011 Annual International Conference of the IEEE Engineering in Medicine and Biology Society 4641-4644 (2011).
70. Tang, X., Shen, H., Zhao, S., Li, N. & Liu, J. Flexible brain-computer interfaces. Nature Electronics 6, 109-118 (2023).
71. Akin, T., Najafi, K., Smoke, R. H. & Bradley, R. M. A micromachined silicon sieve electrode for nerve regeneration applications. IEEE Transactions on Biomedical Engineering 41, 305-313 (1994).
72. Fattahi, P., Yang, G., Kim, G. & Abidian, M. R. A Review of Organic and Inorganic Biomaterials for Neural Interfaces. Advanced Materials 26, 1846-1885 (2014).
73. Nimbalkar, S. et al. Ultra-Capacitive Carbon Neural Probe Allows Simultaneous Long-Term Electrical Stimulations and High-Resolution Neurotransmitter Detection. Scientific Reports 8, 6958 (2018).
74. Jonsson, A. et al. Bioelectronic neural pixel: Chemical stimulation and electrical sensing at the same site. Proceedings of the National Academy of Sciences 113, 9440-9445 (2016).
75. Takeuchi, S. & Shimoyama, I. A radio-telemetry system with a shape memory alloy microelectrode for neural recording of freely moving insects. IEEE Transactions on Biomedical Engineering 51, 133-137 (2004).
76. Pang, S., Hernandez, Y., Feng, X. & Müllen, K. Graphene as transparent electrode material for organic electronics. Advanced Materials 23, 2779-2795 (2011).
77. Koo, J. et al. Wireless bioresorbable electronic system enables sustained nonpharmacological neuroregenerative therapy. Nature Medicine 24, 1830-1836 (2018).
78. Cogan, S. F. Neural Stimulation and Recording Electrodes. Annual Review of Biomedical Engineering 10, 275-309 (2008).
79. Polikov, V. S., Tresco, P. A. & Reichert, W. M. Response of brain tissue to chronically implanted neural electrodes. Journal of Neuroscience Methods 148, 1-18 (2005).
80. Xie, C. et al. Three-dimensional macroporous nanoelectronic networks as minimally invasive brain probes. Nature Materials 14, 1286-1292 (2015).
81. Cho, S. H. et al. Biocompatible SU-8-Based Microprobes for Recording Neural Spike Signals From Regenerated Peripheral Nerve Fibers. IEEE Sensors Journal 8, 1830-1836 (2008).
82. Fekete, Z. Recent advances in silicon-based neural microelectrodes and microsystems: a review. Sensors and Actuators B: Chemical 215, 300-315 (2015).
83. Liu, Y. et al. Soft and elastic hydrogel-based microelectronics for localized low-voltage neuromodulation. Nature Biomedical Engineering 3, 58-68 (2019).
84. Lecomte, A., Descamps, E. & Bergaud, C. A review on mechanical considerations for chronically-implanted neural probes. Journal of Neural Engineering 15, 031001 (2018).
85. Park, S. et al. Adaptive and multifunctional hydrogel hybrid probes for long-term sensing and modulation of neural activity. Nature Communications 12, 3435 (2021).
86. Ximiao, W., Tingyi, L. & Pei-Yu, C. in 2016 International Conference on Optical MEMS and Nanophotonics (OMN) 1-2 (2016).
87. Kook, G., Lee, S. W., Lee, H. C., Cho, I.-J. & Lee, H. J. in Micromachines, Vol. 7 (2016).
88. Ward, P. A. & Lentsch, A. B. The Acute Inflammatory Response and Its Regulation. Archives of Surgery 134, 666-669 (1999).
89. Campbell, A. & Wu, C. in Micromachines, Vol. 9 (2018).
90. Karumbaiah, L. et al. Relationship between intracortical electrode design and chronic recording function. Biomaterials 34, 8061-8074 (2013).
91. Lacour, S.p.P., Courtine, G. & Guck, J. R. Materials and technologies for soft implantable neuroprostheses. Nature Reviews Materials 1 (2016).
92. Wassie, A. T., Zhao, Y. & Boyden, E. S. Expansion microscopy: principles and uses in biological research. Nature Methods 16, 33-41 (2019).
93. Richardson, D. S. et al. Tissue clearing. Nature Reviews Methods Primers 1, 84 (2021).
94. Li, H. et al. Ultrastretchable and superior healable supercapacitors based on a double cross-linked hydrogel electrolyte. Nature Communications 10, 536 (2019).
95. Lin, S. et al. Anti-fatigue-fracture hydrogels. Science Advances 5, eaau8528.
96. Maitra, J. & Shukla, V. K. Cross-linking in hydrogels-a review. Am. J. Polym. Sci 4, 25-31 (2014).
97. Gacesa, P. Enzymic degradation of alginates. International Journal of Biochemistry 24, 545-552 (1992).
98. Goosen, M. F. A., O'Shea, G. M., Gharapetian, H. M., Chou, S. & Sun, A. M. Optimization of microencapsulation parameters: Semipermeable microcapsules as a bioartificial pancreas. Biotechnology and Bioengineering 27, 146-150 (1985).
99. Gombotz, W. R. & Wee, S. F. Protein release from alginate matrices. Advanced Drug Delivery Reviews 64, 194-205 (2012).
100. Chenite, A. et al. Novel injectable neutral solutions of chitosan form biodegradable gels in situ. Biomaterials 21, 2155-2161 (2000).
101. Watanabe, T., Ohtsuka, A., Murase, N., Barth, P. & Gersonde, K. NMR studies on water and polymer diffusion in dextran gels. Influence of potassium ions on microstructure formation and gelation mechanism. Magnetic Resonance in Medicine 35, 697-705 (1996).
102. Janes, K. A., Fresneau, M. P., Marazuela, A., Fabra, A. & Alonso, M.a.J. Chitosan nanoparticles as delivery systems for doxorubicin. Journal of Controlled Release 73, 255-267 (2001).
103. Peppas, N. A. & Mongia, N. K. Ultrapure poly(vinyl alcohol) hydrogels with mucoadhesive drug delivery characteristics. European Journal of Pharmaceutics and Biopharmaceutics 43, 51-58 (1997).
104. Hatakeyema, T., Uno, J., Yamada, C., Kishi, A. & Hatakeyama, H. Gel-sol transition of poly(vinyl alcohol) hydrogels formed by freezing and thawing. Thermochimica Acta 431, 144-148 (2005).
105. Hennink, W. E. & van Nostrum, C. F. Novel crosslinking methods to design hydrogels. Advanced Drug Delivery Reviews 64, 223-236 (2012).
106. Zu, Y. et al. Preparation and characterization of chitosan-polyvinyl alcohol blend hydrogels for the controlled release of nano-insulin. International Journal of Biological Macromolecules 50, 82-87 (2012).
107. Froidevaux, V., Negrell, C., Caillol, S., Pascault, J.-P. & Boutevin, B. Biobased Amines: From Synthesis to Polymers; Present and Future. Chemical Reviews 116, 14181-14224 (2016).
108. Patil, G. V. Biopolymer albumin for diagnosis and in drug delivery. Drug Development Research 58, 219-247 (2003).
109. Fatima, R. et al. Aminated Polysaccharides: Unveiling a new frontier for enhanced therapeutic efficacy. Journal of Drug Delivery Science and Technology 89, 105090 (2023).
110. Pederson, A. W., Ruberti, J. W. & Messersmith, P. B. Thermal assembly of a biomimetic mineral/collagen composite. Biomaterials 24, 4881-4890 (2003).
111. Xiao, W. et al. Ultrasonication and Genipin Cross-Linking to Prepare Novel Silk Fibroin-Gelatin Composite Hydrogel. Journal of Bioactive and Compatible Polymers 27, 327-341 (2012).
112. Andreopoulos, F. M., Beckman, E. J. & Russell, A. J. Light-induced tailoring of PEG-hydrogel properties. Biomaterials 19, 1343-1352 (1998).
113. Jacques, S. L. Optical properties of biological tissues: a review. Physics in Medicine & Biology 58, R37 (2013).
114. Umar, M., Min, K. & Kim, S. Advances in hydrogel photonics and their applications. APL Photonics 4, 120901 (2019).
115. Guo, J. et al. Highly Stretchable, Strain Sensing Hydrogel Optical Fibers. Advanced Materials 28, 10244-10249 (2016).
116. Wang, L. et al. Ultrasoft and Highly Stretchable Hydrogel Optical Fibers for In Vivo Optogenetic Modulations. Advanced Optical Materials 6, 1800427 (2018).
117. Yun, S. H. & Kwok, S. J. J. Light in diagnosis, therapy and surgery. Nature Biomedical Engineering 1, 0008 (2017).
118. Choi, M. et al. Light-guiding hydrogels for cell-based sensing and optogenetic synthesis in vivo. Nature Photonics 7, 987-994 (2013).
119. Choi, M., Humar, M., Kim, S. & Yun, S.-H. Step-Index Optical Fiber Made of Biocompatible Hydrogels. Advanced Materials 27, 4081-4086 (2015).
120. Zhang, Q. et al. High Refractive Index Inorganic-Organic Interpenetrating Polymer Network (IPN) Hydrogel Nanocomposite toward Artificial Cornea Implants. ACS Macro Letters 1, 876-881 (2012).
121. Lee, C.-J. et al. Ionic Conductivity of Polyelectrolyte Hydrogels. ACS Applied Materials & Interfaces 10, 5845-5852 (2018).
122. Dechiraju, H., Jia, M., Luo, L. & Rolandi, M. Ion-Conducting Hydrogels and Their Applications in Bioelectronics. Advanced Sustainable Systems 6, 2100173 (2022).
123. Zhou, Y. et al. Highly Stretchable, Elastic, and Ionic Conductive Hydrogel for Artificial Soft Electronics. Advanced Functional Materials 29, 1806220 (2019).
124. Keplinger, C. et al. Stretchable, Transparent, Ionic Conductors. Science 341, 984-987 (2013).
125. Li, Y. in Organic Optoelectronic Materials. (ed. Y. Li) 23-50 (Springer International Publishing, Cham; 2015).
126. Awuzie, C. I. Conducting Polymers. Materials Today: Proceedings 4, 5721-5726 (2017).
127. Cayre, O. J., Chang, S. T. & Velev, O. D. Polyelectrolyte Diode: Nonlinear Current Response of a Junction between Aqueous Ionic Gels. Journal of the American Chemical Society 129, 10801-10806 (2007).
128. Koo, H.-J., Chang, S. T., Slocik, J. M., Naik, R. R. & Velev, O. D. Aqueous soft matter based photovoltaic devices. Journal of Materials Chemistry 21, 72-79 (2011).
129. Schroeder, T. B. H. et al. An electric-eel-inspired soft power source from stacked hydrogels. Nature 552, 214-218 (2017).
130. Gao, Y. et al. Hydrogel microphones for stealthy underwater listening. Nature Communications 7, 12316 (2016).
131. Mottet, L. et al. A conductive hydrogel based on alginate and carbon nanotubes for probing microbial electroactivity. Soft Matter 14, 1434-1441 (2018).
132. Jo, H. et al. Electrically conductive graphene/polyacrylamide hydrogels produced by mild chemical reduction for enhanced myoblast growth and differentiation. Acta Biomaterialia 48, 100-109 (2017).
133. Park, J.-E. et al. Rewritable, Printable Conducting Liquid Metal Hydrogel. ACS Nano 13, 9122-9130 (2019).
134. Grieder, W. P., Gannett, W. & Saunders, R. Percolation in Carbon Nanotube Networks. IdeaFest: Interdisciplinary Journal of Creative Works and Research from Cal Poly Humboldt 4, 10 (2020).
135. Zeng, X. et al. Characteristics of the Electrical Percolation in Carbon Nanotubes/Polymer Nanocomposites. The Journal of Physical Chemistry C 115, 21685-21690 (2011).
136. Bauhofer, W. & Kovacs, J. Z. A review and analysis of electrical percolation in carbon nanotube polymer composites. Composites Science and Technology 69, 1486-1498 (2009).
137. Xu, Y. et al. Phase-separated porous nanocomposite with ultralow percolation threshold for wireless bioelectronics. Nature Nanotechnology (2024).
138. Green, R. & Abidian, M. R. Conducting Polymers for Neural Prosthetic and Neural Interface Applications. Advanced Materials 27, 7620-7637 (2015).
139. Green, R. A., Lovell, N. H., Wallace, G. G. & Poole-Warren, L. A. Conducting polymers for neural interfaces: Challenges in developing an effective long-term implant. Biomaterials 29, 3393-3399 (2008).
140. Bhat, M. A., Rather, R. A. & Shalla, A. H. PEDOT and PEDOT: PSS conducting polymeric hydrogels: A report on their emerging applications. Synthetic Metals 273, 116709 (2021).
141. Lu, B. et al. Pure PEDOT: PSS hydrogels. Nature Communications 10, 1043 (2019).
142. Shi, Y., Ma, C., Peng, L. & Yu, G. Conductive “Smart” Hybrid Hydrogels with PNIPAM and Nanostructured Conductive Polymers. Advanced Functional Materials 25, 1219-1225 (2015).
143. Pan, L. et al. Hierarchical nanostructured conducting polymer hydrogel with high electrochemical activity. Proceedings of the National Academy of Sciences 109, 9287-9292 (2012).
144. Zhao, F., Shi, Y., Pan, L. & Yu, G. Multifunctional Nanostructured Conductive Polymer Gels: Synthesis, Properties, and Applications. Accounts of Chemical Research 50, 1734-1743 (2017).
145. Langer, R. Drug delivery and targeting. Nature 392, 5-10 (1998).
146. Cohen, J. IL-12 Deaths: Explanation and a Puzzle. Science 270, 908-908 (1995).
147. Florence, A. T. & Jani, P. U. Novel Oral Drug Formulations. Drug Safety 10, 233-266 (1994).
148. Ashley, G. W., Henise, J., Reid, R. & Santi, D. V. Hydrogel drug delivery system with predictable and tunable drug release and degradation rates. Proceedings of the National Academy of Sciences 110, 2318-2323 (2013).
149. Emiliani, V. et al. Optogenetics for light control of biological systems. Nature Reviews Methods Primers 2, 55 (2022).
150. Liu, X. et al. Fatigue-resistant hydrogel optical fibers enable peripheral nerve optogenetics during locomotion. Nature Methods 20, 1802-1809 (2023).
151. Du, M. et al. Flexible Fiber Probe for Efficient Neural Stimulation and Detection. Advanced Science 7, 2001410 (2020).
152. Sui, K. et al. Adaptive polymer fiber neural device for drug delivery and enlarged illumination angle for neuromodulation. Journal of Neural Engineering 19, 016035 (2022).
153. Park, S., Loke, G., Fink, Y. & Anikeeva, P. Flexible fiber-based optoelectronics for neural interfaces. Chemical Society Reviews 48, 1826-1852 (2019).
154. Song, E., Li, J., Won, S. M., Bai, W. & Rogers, J. A. Materials for flexible bioelectronic systems as chronic neural interfaces. Nature Materials 19, 590-603 (2020).
155. Chen, F., Tillberg, P. W. & Boyden, E. S. Expansion microscopy. Science 347, 543-548 (2015).
156. Ueda, H. R. et al. Tissue clearing and its applications in neuroscience. Nature Reviews Neuroscience 21, 61-79 (2020).
157. Huang, S. et al. Control of polymers' amorphous-crystalline transition enables miniaturization and multifunctional integration for hydrogel bioelectronics. Nature Communications 15, 3525 (2024).
158. Lin, S., Liu, J., Liu, X. & Zhao, X. Muscle-like fatigue-resistant hydrogels by mechanical training. Proceedings of the National Academy of Sciences 116, 10244-10249 (2019).
159. Higashihara, T. & Ueda, M. Recent Progress in High Refractive Index Polymers. Macromolecules 48, 1915-1929 (2015).
160. Tarcan, R. et al. Reduced graphene oxide today. Journal of Materials Chemistry C 8, 1198-1224 (2020).
161. Javadi, M. et al. Conductive Tough Hydrogel for Bioapplications. Macromolecular Bioscience 18, 1700270 (2018).
162. Yang, C. & Suo, Z. Hydrogel ionotronics. Nature Reviews Materials 3, 125-142 (2018).
163. Guimarães, C. F., Gasperini, L., Marques, A. P. & Reis, R. L. The stiffness of living tissues and its implications for tissue engineering. Nature Reviews Materials 5, 351-370 (2020).
164. Yang, S. et al. Stretchable surface electromyography electrode array patch for tendon location and muscle injury prevention. Nature Communications 14, 6494 (2023).
165. Li, Y. et al. Achieving tissue-level softness on stretchable electronics through a generalizable soft interlayer design. Nature Communications 14, 4488 (2023).
166. Liu, S., Rao, Y., Jang, H., Tan, P. & Lu, N. Strategies for body-conformable electronics. Matter 5, 1104-1136 (2022).
167. Yoon, Y. et al. Neural probe system for behavioral neuropharmacology by bi-directional wireless drug delivery and electrophysiology in socially interacting mice. Nature Communications 13, 5521 (2022).
168. Zhou, T. et al. 3D printable high-performance conducting polymer hydrogel for all-hydrogel bioelectronic interfaces. Nature Materials 22, 895-902 (2023).
169. Xing, Y. et al. Integrated opposite charge grafting induced ionic-junction fiber. Nature Communications 14, 2355 (2023).
170. Yang, J., Webb, A. R. & Ameer, G. A. Novel Citric Acid-Based Biodegradable Elastomers for Tissue Engineering. Advanced Materials 16, 511-516 (2004).
171. Shen, K., Chen, O., Edmunds, J. L., Piech, D. K. & Maharbiz, M. M. Translational opportunities and challenges of invasive electrodes for neural interfaces. Nature Biomedical Engineering 7, 424-442 (2023).
172. Lu, C. et al. Polymer Fiber Probes Enable Optical Control of Spinal Cord and Muscle Function In Vivo. Advanced Functional Materials 24, 6594-6600 (2014).
173. Djordjevic, I., Choudhury, N. R., Dutta, N. K. & Kumar, S. Poly [octanediol-co-(citric acid)-co-(sebacic acid)] elastomers: novel bio-elastomers for tissue engineering. Polymer International 60, 333-343 (2011).
174. Sundback, C. A. et al. Biocompatibility analysis of poly(glycerol sebacate) as a nerve guide material. Biomaterials 26, 5454-5464 (2005).
175. Gilletti, A. & Muthuswamy, J. Brain micromotion around implants in the rodent somatosensory cortex. Journal of Neural Engineering 3, 189 (2006).
176. Sahasrabudhe, A. et al. Multifunctional microelectronic fibers enable wireless modulation of gut and brain neural circuits. Nature Biotechnology 42, 892-904 (2024).
177. Kurkjian, C. R., Krause, J. T. & Matthewson, M. J. Strength and fatigue of silica optical fibers. Journal of Lightwave Technology 7, 1360-1370 (1989).
178. Király, B. et al. In vivo localization of chronically implanted electrodes and optic fibers in mice. Nature Communications 11, 4686 (2020).
179. Nguyen, J. K. et al. Mechanically-compliant intracortical implants reduce the neuroinflammatory response. Journal of Neural Engineering 11, 056014 (2014).
180. Serruya, M. D. et al. Engineered Axonal Tracts as “Living Electrodes” for Synaptic-Based Modulation of Neural Circuitry. Advanced Functional Materials 28, 1701183 (2018).
181. Park, S. et al. One-step optogenetics with multifunctional flexible polymer fibers. Nature Neuroscience 20, 612-619 (2017).
182. Garwood, I. C. et al. Multifunctional fibers enable modulation of cortical and deep brain activity during cognitive behavior in macaques. Science Advances 9, eadh0974.
183. David-Pur, M., Bareket-Keren, L., Beit-Yaakov, G., Raz-Prag, D. & Hanein, Y. All-carbon-nanotube flexible multi-electrode array for neuronal recording and stimulation. Biomedical Microdevices 16, 43-53 (2014).
184. Maruoka, S. et al. Biocompatibility of Polyvinylalcohol Gel as a Vitreous Substitute. Current Eye Research 31, 599-606 (2006).
185. Eban-Rothschild, A., Rothschild, G., Giardino, W. J., Jones, J. R. & de Lecea, L. VTA dopaminergic neurons regulate ethologically relevant sleep-wake behaviors. Nature Neuroscience 19, 1356-1366 (2016).
186. Nath, T. et al. Using DeepLabCut for 3D markerless pose estimation across species and behaviors. Nature Protocols 14, 2152-2176 (2019).
187. Kim, C. K. et al. Simultaneous fast measurement of circuit dynamics at multiple sites across the mammalian brain. Nature Methods 13, 325-328 (2016).
188. Schiavone, G. et al. Soft, Implantable Bioelectronic Interfaces for Translational Research. Advanced Materials 32, 1906512 (2020).
189. Fallegger, F., Schiavone, G. & Lacour, S. P. Conformable Hybrid Systems for Implantable Bioelectronic Interfaces. Advanced Materials 32, 1903904 (2020).
190. Jiang, Y. et al. A universal interface for plug-and-play assembly of stretchable devices. Nature 614, 456-462 (2023).
191. Kathe, C. et al. Wireless closed-loop optogenetics across the entire dorsoventral spinal cord in mice. Nature Biotechnology 40, 198-208 (2022).
192. Liu, Y. et al. Morphing electronics enable neuromodulation in growing tissue. Nature Biotechnology 38, 1031-1036 (2020).
193. Fan, J. A. et al. Fractal design concepts for stretchable electronics. Nature Communications 5, 3266 (2014).
194. Gerratt, A. P., Michaud, H. O. & Lacour, S. P. Elastomeric Electronic Skin for Prosthetic Tactile Sensation. Advanced Functional Materials 25, 2287-2295 (2015).
195. Choi, S., Han, S. I., Kim, D., Hyeon, T. & Kim, D.-H. High-performance stretchable conductive nanocomposites: materials, processes, and device applications. Chemical Society Reviews 48, 1566-1595 (2019).
196. Chen, R. et al. Highly stretchable and fatigue resistant hydrogels with low Young's modulus as transparent and flexible strain sensors. Journal of Materials Chemistry C 6, 11193-11201 (2018).
197. Wang, T., Liu, Q., Liu, H., Xu, B. & Xu, H. Printable and Highly Stretchable Viscoelastic Conductors with Kinematically Reconstructed Conductive Pathways. Advanced Materials 34, 2202418 (2022).
198. Jung, D. et al. Adaptive Self-Organization of Nanomaterials Enables Strain-Insensitive Resistance of Stretchable Metallic Nanocomposites. Advanced Materials 34, 2200980 (2022).
199. Kougkolos, G., Golzio, M., Laudebat, L., Valdez-Nava, Z. & Flahaut, E. Hydrogels with electrically conductive nanomaterials for biomedical applications. Journal of Materials Chemistry B 11, 2036-2062 (2023).
200. Chung, H. U. et al. Skin-interfaced biosensors for advanced wireless physiological monitoring in neonatal and pediatric intensive-care units. Nature Medicine 26, 418-429 (2020).
201. Kim, J., Campbell, A. S., de Avila, B. E.-F. & Wang, J. Wearable biosensors for healthcare monitoring. Nature Biotechnology 37, 389-406 (2019).
202. Zhang, B. et al. A three-dimensional liquid diode for soft, integrated permeable electronics. Nature 628, 84-92 (2024).
203. Oyen, M. L. Mechanical characterisation of hydrogel materials. International Materials Reviews 59, 44-59 (2014).
204. Bai, R. et al. Fatigue fracture of tough hydrogels. Extreme Mechanics Letters 15, 91-96 (2017).
205. Yuan, Y. & Lu, F. A Flexible Chamber for Time-Lapse Live-Cell Imaging with Stimulated Raman Scattering Microscopy. JoVE, e64449 (2022).
206. Lu, F.-K. et al. Label-Free Neurosurgical Pathology with Stimulated Raman Imaging. Cancer Research 76, 3451-3462 (2016).
207. Kim, K. K. et al. Dependence of Raman spectra $ {G} {\ensuremath {′}} $ band intensity on metallicity of single-wall carbon nanotubes. Physical Review B 76, 205426 (2007).
208. Puech, P. et al. Raman bands of double-wall carbon nanotubes: comparison with single- and triple-wall carbon nanotubes, and influence of annealing and electron irradiation. Journal of Raman Spectroscopy 38, 714-720 (2007).
209. Kashiwagi, T. et al. Relationship between dispersion metric and properties of PMMA/SWNT nanocomposites. Polymer 48, 4855-4866 (2007).
210. Sun, X. et al. Carbon nanotubes reinforced hydrogel as flexible strain sensor with high stretchability and mechanically toughness. Chemical Engineering Journal 382, 122832 (2020).
211. Jaspers, M. et al. Nonlinear mechanics of hybrid polymer networks that mimic the complex mechanical environment of cells. Nature Communications 8, 15478 (2017).
212. Kamoun, E. A., Chen, X., Mohy Eldin, M. S. & Kenawy, E.-R. S. Crosslinked poly(vinyl alcohol) hydrogels for wound dressing applications: A review of remarkably blended polymers. Arabian Journal of Chemistry 8, 1-14 (2015).
213. Hukins, D. W. L., Mahomed, A. & Kukureka, S. N. Accelerated aging for testing polymeric biomaterials and medical devices. Medical Engineering & Physics 30, 1270-1274 (2008).
214. de Sousa, R. R., Jr., Heinze, D. A., Sacramento, J. B., Lanfredi, A. J. C. & Carastan, D. J. Electrical Conductivity and In Situ SAXS Probing of Block Copolymer Nanocomposites Under Mechanical Stretching. ACS Applied Materials & Interfaces 15, 27156-27165 (2023).
215. Costa, P., Ribeiro, S., Botelho, G., Machado, A. V. & Lanceros Mendez, S. Effect of butadiene/styrene ratio, block structure and carbon nanotube content on the mechanical and electrical properties of thermoplastic elastomers after UV ageing. Polymer Testing 42, 225-233 (2015).
216. Dohnalová, L. et al. A microbiome-dependent gut-brain pathway regulates motivation for exercise. Nature 612, 739-747 (2022).
217. Kathe, C. et al. The neurons that restore walking after paralysis. Nature 611, 540-547 (2022).
218. Chen, B. et al. Reactivation of Dormant Relay Pathways in Injured Spinal Cord by KCC2 Manipulations. Cell 174, 521-535.e513 (2018).
219. Lemieux, M., Karimi, N. & Bretzner, F. Functional plasticity of glutamatergic neurons of medullary reticular nuclei after spinal cord injury in mice. Nature Communications 15, 1542 (2024).
220. Greiner, N. et al. Recruitment of upper-limb motoneurons with epidural electrical stimulation of the cervical spinal cord. Nature Communications 12, 435 (2021).
221. Milekovic, T. et al. A spinal cord neuroprosthesis for locomotor deficits due to Parkinson's disease. Nature Medicine 29, 2854-2865 (2023).
222. Squair, J. W. et al. Neuroprosthetic baroreflex controls haemodynamics after spinal cord injury. Nature 590, 308-314 (2021).
223. Barra, B. et al. Epidural electrical stimulation of the cervical dorsal roots restores voluntary upper limb control in paralyzed monkeys. Nature Neuroscience 25, 924-934 (2022).
224. Le Floch, P. et al. 3D spatiotemporally scalable in vivo neural probes based on fluorinated elastomers. Nature Nanotechnology 19, 319-329 (2024).
225. Lycke, R. et al. Low-threshold, high-resolution, chronically stable intracortical microstimulation by ultraflexible electrodes. Cell Reports 42, 112554 (2023).
226. Choi, C., Lee, Y., Cho, K. W., Koo, J. H. & Kim, D.-H. Wearable and Implantable Soft Bioelectronics Using Two-Dimensional Materials. Accounts of Chemical Research 52, 73-81 (2019).
227. Tetik, H. et al. 3D Printed MXene Aerogels with Truly 3D Macrostructure and Highly Engineered Microstructure for Enhanced Electrical and Electrochemical Performance. Advanced Materials 34, 2104980 (2022).
228. Qiu, K. & Wegst, U. G. K. Excellent Specific Mechanical and Electrical Properties of Anisotropic Freeze-Cast Native and Carbonized Bacterial Cellulose-Alginate Foams. Advanced Functional Materials 32, 2105635 (2022).
229. Cui, Y. et al. Smart Sponge for Fast Liquid Absorption and Thermal Responsive Self-Squeezing. Advanced Materials 32, 1908249 (2020).
230. Yu, Z.-L. et al. Bioinspired polymeric woods. Science Advances 4, eaat7223.
231. Li, D., Bu, X., Xu, Z., Luo, Y. & Bai, H. Bioinspired Multifunctional Cellular Plastics with a Negative Poisson's Ratio for High Energy Dissipation. Advanced Materials 32, 2001222 (2020).
232. Wu, S. et al. Poly(vinyl alcohol) Hydrogels with Broad-Range Tunable Mechanical Properties via the Hofmeister Effect. Advanced Materials 33, 2007829 (2021).
233. Hua, M. et al. Strong tough hydrogels via the synergy of freeze-casting and salting out. Nature 590, 594-599 (2021).
234. Liang, X. et al. Anisotropically Fatigue-Resistant Hydrogels. Advanced Materials 33, 2102011 (2021).
235. Bai, H., Chen, Y., Delattre, B., Tomsia, A. P. & Ritchie, R. O. Bioinspired large-scale aligned porous materials assembled with dual temperature gradients. Science Advances 1, e1500849.
236. Cui, Y., Gong, H., Wang, Y., Li, D. & Bai, H. A Thermally Insulating Textile Inspired by Polar Bear Hair. Advanced Materials 30, 1706807 (2018).
237. Deville, S., Saiz, E., Nalla, R. K. & Tomsia, A. P. Freezing as a Path to Build Complex Composites. Science 311, 515-518 (2006).
238. Zhao, N., Li, M., Gong, H. & Bai, H. Controlling ice formation on gradient wettability surface for high-performance bioinspired materials. Science Advances 6, eabb4712.
239. Amsden, B. Solute Diffusion within Hydrogels. Mechanisms and Models. Macromolecules 31, 8382-8395 (1998).
240. Chen, J., Peng, Q., Thundat, T. & Zeng, H. Stretchable, Injectable, and Self-Healing Conductive Hydrogel Enabled by Multiple Hydrogen Bonding toward Wearable Electronics. Chemistry of Materials 31, 4553-4563 (2019).
241. Kauffman, K. J. et al. Rapid, Single-Cell Analysis and Discovery of Vectored mRNA Transfection <em>In& #xa0; Vivo</em>with a loxP-Flanked tdTomato Reporter Mouse. Molecular Therapy Nucleic Acids 10, 55-63 (2018).
242. Holback, H., Yeo, Y. & Park, K. in Biomedical Hydrogels. (ed. S. Rimmer) 3-24 (Woodhead Publishing, 2011).
243. Leskelä, M. & Ritala, M. Atomic layer deposition (ALD): from precursors to thin film structures. Thin Solid Films 409, 138-146 (2002).
244. Magno, L. A. V. et al. Optogenetic Stimulation of the M2 Cortex Reverts Motor Dysfunction in a Mouse Model of Parkinson& #039; s Disease. The Journal of Neuroscience 39, 3234 (2019).
245. Hua, J. et al. in Biosensors, Vol. 13 (2023).
246. Gierej, A. et al. in Materials, Vol. 14 (2021).
247. Li, Y., Xin, H., Zhang, Y. & Li, B. Optical Fiber Technologies for Nanomanipulation and Biodetection: A Review. Journal of Lightwave Technology 39, 251-262 (2021).
248. Zhang, L., Tang, Y. & Tong, L. Micro/Nanofiber Optics: Merging Photonics and Material Science on Nanoscale for Advanced Sensing Technology. iScience 23 (2020).
249. Spitalsky, Z., Tasis, D., Papagelis, K. & Galiotis, C. Carbon nanotube-polymer composites: Chemistry, processing, mechanical and electrical properties. Progress in Polymer Science 35, 357-401 (2010).
250. Schütt, F. et al. Hierarchical self-entangled carbon nanotube tube networks. Nature Communications 8, 1215 (2017).
251. Cuttaz, E. A., Bailey, Z. K., Chapman, C. A. R., Goding, J. A. & Green, R. A. Polymer Bioelectronics: A Solution for Both Stimulating and Recording Electrodes. Advanced Healthcare Materials n/a, 2304447 (2024).
252. Xiong, Z. et al. Harnessing the 2D Structure-Enabled Viscoelasticity of Graphene-Based Hydrogel Membranes for Chronic Neural Interfacing. Small Methods 6, 2200022 (2022).
253. Huang, S., Liu, X., Lin, S. et al. Control of polymers' amorphous-crystalline transition enables miniaturization and multifunctional integration for hydrogel bioelectronics. Nat Communication 15, 3525 (2024).
254. Liu, X., Rao, S., . . . , Huang, S. et al. Fatigue-resistant hydrogel optical fibers enable peripheral nerve optogenetics during locomotion. Nat Methods 20, 1802-1809 (2023).
255. Xiao, R. f, Wu, Z. †, Huang, S. et al. Gradient porous hydrogel fibers for spinal drug delivery and pain inhibition. In preparation.
256. Liu, X.f, Rao, S.f, Huang, S. et al. Multifunctional soft neural probes for integrated chemical delivery and optical interrogation. In preparation.
257. Huang, S., Villafranca, S., Mehta, I., et al. A nanoscale inorganic coating strategy for stabilizing hydrogel neural probes in vivo. Journal of Materials Chemistry B 11, no. 32 (2023): 7629-7640.
258. Huang, S., et al. Flexible and stretchable functional layer coated fibers for pain management via optoelectronic probing in spinal cord circuits. To be submitted.
259. Huang, S., Liu, X., Lin, S. et al. Control of polymers' amorphous-crystalline transition enables miniaturization and multifunctional integration for hydrogel bioelectronics. Nat Communication 15, 3525 (2024).
260. Rao, S, Huang, S., Wang, Q., et al. “Miniaturized hydrogel and uses thereof.” U. S. Pub. Pat Appln. 2024/0269352.
261. Huang, S., et al. Flexible and stretchable functional layer coated fibers for pain management via optoelectronic probing in spinal cord circuits. To be submitted.
262. Lu, Chi, “Flexible Fibers for Optoelectronic Probing of Spinal Cord Circuits”, MIT Ph.D Dissertation (2017).
263. Jiang, Shan. “Multimaterial multifunctional fibers for biomedical applications.” Virginia Polytechnic Institute Ph.D. dissertation (2021).
264. Park, S.-I.; Brenner, D. S.; Shin, G.; Morgan, C. D.; Copits, B. A.; Chung, H. U.; Pullen, M. Y.; Noh, K. N.; Davidson, S.; Oh, S. J.; et al. Soft, stretchable, fully implantable miniaturized optoelectronic systems for wireless optogenetics. Nat. Biotechnol. 2015, 33, 1280-1286.
265. Zhang, Hui, Yaping Liu, Kai Zhou, Wei Wei, and Yaobo Liu. “Restoring sensorimotor function through neuromodulation after spinal cord injury: progress and remaining challenges.” Frontiers in Neuroscience 15 (2021): 749465.
266. Lacour, Stéphanie P., Samia Benmerah, Edward Tarte, James FitzGerald, Jordi Serra, Stephen McMahon, James Fawcett, Oliver Graudejus, Zhe Yu, and Barclay Morrison. “Flexible and stretchable micro-electrodes for in vitro and in vivo neural interfaces.” Medical & biological engineering & computing 48 (2010): 945-954.
267. Fan, Xi, Wanyi Nie, Hsinhan Tsai, Naixiang Wang, Huihui Huang, Yajun Cheng, Rongjiang Wen, Liujia Ma, Feng Yan, and Yonggao Xia. “PEDOT:PSS for flexible and stretchable electronics: modifications, strategies, and applications.” Advanced Science 6, no. 19 (2019): 1900813.

What is claimed is:

1. A hydrogel with a chemically crosslinked polymer matrix, having conductive nanorods or nanotubes.

2. The hydrogel according to claim 1, wherein the hydrogel is semi-crystalline.

3. The hydrogel according to claim 1, wherein the crosslinked polymer matrix comprises a polymer selected from the group consisting of: polyvinyl alcohol, poly(3,4-ethylenedioxythiophene), polystyrene sulfonate (PEDOT:PSS), Polydimethylsiloxane (PDMS), Styrene-Ethylene-Butylene-Styrene (SEBS), Poly(1,8-octanediol-co-citric acid) (POC), Polyimide (PI), Polycarbonate (PC) and Cyclic olefin copolymer (COC), Poly(etherimide) (PEI), epoxy-based negative photoresist, and Poly(glycolic acid) (PGA).

4. (canceled)

5. The hydrogel according to claim 1, wherein the conductive nanorods or nanotubes are anisotropically aligned.

6. The hydrogel according to claim 1, wherein the hydrogel is configured as an elongated structure, and wherein the conductive nanorods or nanotubes are axially aligned with the elongated structure.

7. The hydrogel according to claim 1, wherein the chemically crosslinked polymer matrix is soft and stretchable.

8. The hydrogel according to claim 1, displaying anisotropic conductivity.

9-11. (canceled)

12. The hydrogel according to claim 1, wherein the crosslinked polymer matrix has an elastic modulus of between 2.8 to 9.3 MPa, between 3 to 7 MPa, or between 4 to 6 MPa.

13-14. (canceled)

15. The hydrogel according to claim 1, wherein the crosslinked polymer matrix has a stretchability of at least 50%, at least 65%, at least 85%, at least 100%, at least 125%, at least 139%, at least 150%, or at least 168%.

16-22. (canceled)

23. The hydrogel according to claim 1, wherein the crosslinked polymer matrix has a bending stiffness of less than 6 N/m, less than 5 N/m, or less than 4.6 N/m.

24-25. (canceled)

26. The hydrogel according to claim 1, wherein the polymer matrix comprises Poly(vinyl) alcohol crosslinked with Glutaraldehyde.

27. The hydrogel according to claim 1, wherein the polymer matrix is formed from poly(vinyl) alcohol having a molecular weight from 146,000 to 186,000 Da and which is at least 99% hydrolyzed.

28. The hydrogel according to claim 1, configured as a rod having a diameter of less than 500 μm.

29. The hydrogel according to claim 28, having an outer coating of silicon dioxide or styrene-ethylene-butylene-styrene (SEBS).

30-32. (canceled)

33. The hydrogel according to claim 28, having an inner tubular conduit or an inner fiber optic.

34. (canceled)

35. The hydrogel according to claim 28, wherein the hydrogel exhibits ionic conductivity.

36. The hydrogel according to claim 28, in combination with at least one other hydrogel configured as a rod having a diameter of less than 500 μm, less than 400 μm, less than 300 μm, less than 200 μm, less than 100 μm, less than 90 μm, or less than 80 μm, and wherein each rod is electrically insulated from each other rod.

37-42. (canceled)

43. The hydrogel according to claim 28, wherein the hydrogel is an electrode for electrically interfacing with animal tissue, the hydrogel having anisotropically aligned conductive nanorods or nanotubes, surrounded by a tubular insulating layer.

44-45. (canceled)

46. The hydrogel electrode according to claim 43, having
(i) a diameter of less than 250 μm, a stretchability of at least 50% and an impedance of less than 10 kΩ·mm @ 1 kHz;
(ii) a diameter of less than 225 μm, and an impedance of less than 5 kΩ·mm @ 1 kHz;
(iii) a diameter of less than 200 μm, and an impedance of less than 4 kΩ·mm @ 1 kHz; or
(iv) a diameter of less than 100 μm, and an impedance of less than 5 kΩ·mm @ 1 kHz.

47-96. (canceled)

97. A method of making a soft, elastic, conductive hydrogel electrode, comprising:
combining a hydrogel-forming polymer with carbon nanotubes and a carbon nanotube dispersing agent to form a heterogeneous mixture;
crosslinking the hydrogel-forming polymer in a mold to form an elongated hydrogel having a diameter less than 1 mm;
demolding the elongated hydrogel; and
applying a cyclic strain on the molded hydrogel to align the carbon nanotubes with an elongation axis of the molded hydrogel to form an anisotropically-conductive hydrogel.

98-115. (canceled)
